(19) United States
(12) Patent Application Publication
(10) Pub. No.: US 2021/0207221 A1
(43) Pub. Date: Jul. 8, 2021

(54) THE KIT FOR SCREENING COLORECTAL CANCER AND ADVANCED ADENOMA AND ITS APPLICATION

(71) Applicant: Hangzhou New Horizon Health Technology Co. Ltd., Binjiang District, Hangzhou (CN)

(72) Inventors: Cunyao LI, Binjiang District, Hangzhou (CN); Hui LI, Binjiang District, Hangzhou (CN); Weixian ZHENG, Binjiang District, Hangzhou (CN); Jiao YANG, Binjiang District, Hangzhou (CN); Gang LIU, Binjiang District, Hangzhou (CN); Ning LU, Binjiang District, Hangzhou (CN); Yiyou CHEN, Binjiang District, Hangzhou (CN)

(21) Appl. No.: 17/057,227

(22) PCT Filed: May 23, 2019
(86) PCT No.: PCT/US2019/033831

§ 371 Date: Nov. 20, 2020

(30) Foreign Application Priority Data
May 23, 2018 (CN) 201810502359.7
May 23, 2018 (CN) 201810502387.9

(51) Int. Cl. C12Q 1/6886; G01N 33/72

(52) U.S. Cl. CPC C12Q 1/6886; C12Q 2600/156; C12Q 2600/154; G01N 33/721

(57) ABSTRACT

The present inventions provides combinations of primers and probes for performing quantitative PCRs that can be used to determine the methylation state and level of BMP3 gene and NDRG4 genes in a patient in need thereof, which leads to surprisingly high diagnostic specificity and sensitivity for diagnosing the presence or the absence of colorectal cancer (CRC) and/or advanced adenoma (AA) in a patient in need therefore. Compositions and methods for performing the diagnosis are provided.

### CROSS-REFERENCE TO RELATED APPLICATIONS

This application claims priority to Chinese Patent Application Serial Nos. 201810502359.7, filed May 23, 2018, and 201810502387.9, filed May 23, 2018, each of which is herein incorporated by reference in its entirety for all purposes.

### FIELD OF THE INVENTION

The present invention relates to compositions and methods for screening colorectal cancer and advanced adenoma, and other applications.

### DESCRIPTION OF TEXT FILE SUBMITTED ELECTRONICALLY

The contents of the text file submitted electronically herewith are incorporated herein by reference in their entirety: A computer readable format copy of the Sequence Listing (filename: NEWH-017_01WO_SeqList_ST25.txt, date recorded: May 22, 2019, file size: 22 kilobytes).

### BACKGROUND OF THE INVENTION

Colorectal cancer (CRC) is the fourth common cancer in the world, with a mortality only less than lung cancer, liver cancer and stomach cancer. The annual death caused by CRC is nearly 700,000. CRC is a “modernized” disease with a higher incidence in developed countries compared to that in developing countries. In the United States, colorectal cancer remains the second leading cause of death (Clinical Interventions in Aging 2016; 11:967-976). With the improvement of the living standards of people, the incidence and mortality of CRC have been increasing in China since 2000 (CA CANCER J CLIN 2016; 66:115-132). The 5-year survival rates of the patients with early-stage, localized disease (stage I and II) approach 90%, while the survival rate of those with late-stage CRC is only 13.1%. The cost of treatment for patients with late-stage CRC often is enormous and can only alleviate the symptoms of the disease (Clinical Interventions in Aging 2016; 11:967-976).

The development of CRC is a slow process which is generally asymptomatic and difficult to be detected at the early stage until the tumor grows to a few centimeters in size, which may block the passage of feces and lead to cramping, pain or visible bleeding. The development of CRC has gone through multistep process involving a series of histological, morphological, and genetic changes that accumulate over time: namely, from health, hyperplasia, small polyps, large polyps, and adenocarcinoma to cancer. Polyps are abnormal cells that grow or accumulate locally within the intestinal mucosa. The dividing cells in the polyps may accumulate enough genetic changes to penetrate the intestinal wall and eventually evolve into CRC. However, only a small number of polyps have evolved into CRC after more than a decade of development. Two main types of the malignant potential polyps are adenomas and sessile serrated polyps (SSPs), each of which developed into CRC with different risks. The risk of adenoma developing into CRC is related to its size. Generally, Adenomas are larger in size and have a greater potential for developing into CRC. Advanced adenomas (AA) refer to ≥1 cm in size or with ≥25% villous component or high-grade dysplasia of any size. Although only about 10% of the most AA become cancerous, 60%-70% of CRC develop from adenomas, the remaining 25%-35% of CRCs develop from SSPs (Clinical Interventions in Aging 2016; 11:967-976). Therefore, early discovery of CRC and AA, and removal of the lesions, can effectively block the progress of CRC to save the lives of patients, significantly improve the patient's 5-year survival rate, and reduce expensive treatment costs in the late-stage of CRC, which greatly reduces the economic burden of the family and society.

At present, there are several tests for detection of CRC, mainly including colonoscopy, sigmoidoscopy, CT colonography, fecal occult blood test (FOBT) and fecal immunochemical test (FIT).

The sensitivity of colonoscopy for detecting CRC is >95%. Its screening interval is every 10 years. The advantage of colonoscopy is high sensitivity, which can inspect the entire colon and remove the lesion at the same time. However, the disadvantage of which is invasive examination and bowel preparation will bring discomfort, and the patient needs to be calmed. There is a risk of bowel perforation and bleeding during colonoscopy. These limitations contribute to low compliance with colonoscopy screening.

The sensitivity of sigmoidoscopy to detect distal colon is greater than 95%. The screening interval of CRC with sigmoidoscopy is every 5 years in combination with FOBT. The advantage of screening CRC with sigmoidoscopy is high sensitivity, no need for systemic sedation, and the lesion can be removed simultaneously during the examination. The disadvantage of which is semi-invasive examination, easily causing discomfort during examination and the inspection cost is high.

CT colonography uses radiation to visualize the colon, which sensitivity is >90% and performed every 5 years. The advantage of which is so high sensitivity that the entire colon can be observed and no sedation is needed. The disadvantage is that the assay is a semi-invasive examination so that patients will easily feel uncomfortable during the screening process. In addition, the lesions cannot be removed at the same time and radiation safety needs to be considered.

Overview, the tests above to detect CRC based on imaging has high sensitivity, but they are expensive and the bowel preparation is easy to cause discomfort and other side effects. As a result, the patient compliance is low. Additionally, these assays require professional equipment, and doctors with professional skills and rich experience, which may not be available. As a result, the overall screening/detecting rate is low. In addition, some patients are not suitable for these assays. For example, patients with diabetics have a lower success rate of bowel preparation and higher risk of side effects (J Gastrointestin Liver Dis 2010; 19: 369-372, World J Gastrointest Endosc 2013; 5: 39-46).

FOBT and FIT detect hemoglobin in feces of patients by an enzyme reaction and an immunochemical method respectively, with a sensitivity of 33%-75% and 60%-85% respectively for CRC detection, and the tests are performed every 1 year. Although FOBT and FIT are easy to popularize, noninvasive and low cost, the detection rate of precancerous lesions is low (Clinical Interventions in Aging 2016; 11967-976).

During the development of polyps into CRC, mutation and methylation changes in some genes such as APC, KRAS, p53, BRAF, NDRG4, BMP3, etc. are accumulated (Clinical Interventions in Aging 2016; 11:967-976). Therefore, detecting of these mutations or methylation changes helps to detect of CRC and precancerous lesions.

Zou et al. (Clinical Chemistry 2012; 58: 2375-383) used methylation qPCR to detect methylation levels of BMP3, NDRG4, VIM, and TFPI2 genes in tissue samples. In total 37 cases of CRC tissue samples, 25 adenoma tissue samples and 29 healthy human tissue samples were tested. When the specificity was 95%, the sensitivity of BMP3, NDRG4, VIM and TFPI2 genes for CRC detecting was 84%, 92%, 86%, and 92% respectively, and the sensitivity for adenoma detecting was 68%, 76%, 76%, and 88% respectively. It was shown that the detection of genes methylation in colon cancer tissues has high sensitivity and specificity. However, the tissue sampling method is difficult to be widely used, because the sampling process leads to certain damage to the patient's body. Therefore, it is not suitable for screening CRC and precancerous lesions in the general population.

Multitarget stool DNA (mt-sDNA) testing includes methylation and mutation detection of tumor exfoliated cells and hemoglobin detection in stool samples, which is screened every 3 years and has the advantages of high sensitivity, non-invasive and easy to popularize (Clinical Interventions In Aging 2016; 11:967-976). As a screening method, mt-sDNA can detect CRC and AA early which greatly improves the survival rate of patients. Imperiale et al. (N Engl J Med 2014; 370:1287-97) established a system based on mt-sDNA for methylation detection of BMP3 and NDRG4 genes, point mutation detection of KRAS gene and fecal hemoglobin detection, and then assessed the risk of CRC and AA according to a logistic regression formula. The sensitivity of CRC and AA detection was 92.3% and 42.4% respectively, and the specificity was 86.6%.

Mt-sDNA is applied to screen sporadic CRC and AA with a advantage of noninvasive compared to colonoscopy and more sensitive compared to FOBT and FIT, but the sensitivity of AA detection is still far lower than that of CRC (Clinical Interventions in Aging 2016; 11:967-976).

Currently, the products based on mt-sDNA for detecting CRC or AA, such as Cologuard® are mainly developed for European and American populations. No product for CRC and AA detection in the Asian population is available. Particularly, according to “Summary of safety and effectiveness data (SSED)” of Cologuard® issued by the U.S. Food and Drug Administration (www.accessdata.fda.gov/cdrh_docs/pdf13/P30017b.pdf), the sensitity of AA detection using Cologuard® in the white population and the African-American population is 42.3% and 42.4%, respectively, but the sensitivity of AA detection using the same product in the Asian population is only 30.8%. Therefore, there remains a need to develop an effective system for CRC and/or AA detection in the Asian population to cope with the current increase in the incidence and mortality of colorectal cancer in Asian countries.

Although there were many studies on the methods for detecting methylation of BMP3 and NDRG4 genes in stool samples from patients with CRC and AA, there is no detailed and comprehensive researches about the hypermethylated CpG sites in BMP3 and NDRG4 genes of the Asian population (ONCOLOGY LETTERS 2014; 8:1751-1756; ONCOLOGY LETTERS 2015; 9:1383-1387). Moreover, due to limited sample sizes, previous studies of BMP3 and NDRG4 genes methylations in Asian patients were not helpful to identify methylation sites that are most relevant to CRC and AA. Therefore, there remains a need to determine the exact location of the hypermethylated CpG sites of BMP3 and NDRG4 genes in the Asian population, and to design and optimize kits based on these methylated CpG sites, making the detection of AA more sensitive.

### SUMMARY OF THE INVENTION

The present disclosure provides DNA sequences comprising of hypermethylated CpG sites in the promoter region of BMP3 and NDRG4 genes.

The present disclosure also provides preferred primers and probes for detecting methylation of BMP3 or NDRG4 genes, and combinations thereof for detecting methylation of both BMP3 and NDRG4 genes.

The present disclosure further provides a kit for detecting CRC and AA of the Asian population. The DNA sequences comprising of hypermethylated CpG sites in the promoter region of BMP3 and NDRG4 genes can be used as markers for CRC and/or AA detection in Asian population.

Compared to other primers and probes, the pairs of preferred primers and probes for detecting methylation levels of BMP3 and/or NDRG4 genes have surprisingly higher sensitivity and specificity to detect tumor tissue, such as CRC and AA, and especially AA. In addition, the combinations of these preferred primers and probes of the present disclosure also achieve surprisingly higher sensitivity and specificity to detect tumor tissue, such as CRC and AA, and especially AA.

A kit for detecting CRC and AA of Asian population based on above preferred primer and probe combinations is also provided.

In some embodiments, the kit comprises: (1) a preferred combination of pairs of primer and probe and corresponding qPCR reagents; (2) primers and probes for detecting seven mutations ( ) in a coding region of KRAS gene and corresponding qPCR reagents; (3) reagents for detecting hemoglobin in feces.

In some embodiments, results obtained from an assay using the kit are corrected and analyzed according to a logistic regression formula. In some embodiments, the formula is used to calculate a value for determining the presence or absence of CRC and/or AA. In some embodiments, the formula is P=e^{K}/(1+e^{K}), where P is a comprehensive index, and K=a*ΔCt1+b*ΔCt2+c*ΔCt3+d*FIT+X, wherein e is a natural constant, and a, b, c, d, X are clinical constant. In some embodiments, when the P value is equal or more than a predetermined threshold, the result indicates a positive detection of CRC and/or AA in the patient. In some embodiments, when the P value is less than the threshold, the result indicates a negative detection of CRC and/or AA in the patient, and the patient is determined to be healthy.

The present disclosure provides a kit for detecting the presence or the absence of colorectal cancer (CRC) or advanced adenoma (AA) in a patient in need thereof. A patient in need thereof is a patient suspected to have CRC and/or AA, such as a patient having at least one sign of developing CRC and/or AA, or a patient having a risk of developing CRC and/or AA, or a subject having a routine medical checkup but otherwise having no sign or risk.

In some embodiments, the kit comprises a) a first pair of primers and a first probe for detecting the methylation state or level of at least one CpG dinucleotide of the BMP3 gene in a biological sample obtained from the patient. In some embodiments, each of the first pair of primers and first probe comprises a contiguous sequence of at least 16 nucleotides that is identical to, complementary to, or hybridizes under stringent hybridization conditions to SEQ ID NO.: 1,

In some embodiments, the kit comprises b) a second pair of primers and a second probe for detecting the methylation state or level of at least one CpG dinucleotide of the NDRG4 gene in a biological sample obtained from the patient. In some embodiments, each of the second pair of primers and second probe comprises a contiguous sequence of at least 16 nucleotides that is identical to, complementary to, or hybridizes under stringent hybridization conditions to SEQ ID NO.: 2,

In some embodiments, the first pair of primers and the first probe are selected from the group consisting of:

i) a forward primer comprising SEQ ID NO.: 3, a reverse primer comprising SEQ ID NO.: 4, and a probe comprising SEQ ID NO.: 5;

ii) a forward primer comprising SEQ ID NO.: 9, a reverse primer comprising SEQ ID NO.: 10, and a probe comprising SEQ ID NO.: 11; and

iii) a forward primer comprising SEQ ID NO.: 15, a reverse primer comprising SEQ ID NO.: 16, and a probe comprising SEQ ID NO.: 17;

In some embodiments, wherein the second first pair of primers and the second probe are selected from the group consisting of:

iv) a forward primer comprising SEQ ID NO.: 6, a reverse primer comprising SEQ ID NO.: 7, and a probe comprising SEQ ID NO.: 8;

v) a forward primer comprising SEQ ID NO.: 12, a reverse primer comprising SEQ ID NO.: 13, and a probe comprising SEQ ID NO.: 14; and

vi) a forward primer comprising SEQ ID NO.: 18, a reverse primer comprising SEQ ID NO.: 19, and a probe comprising SEQ ID NO.: 20;

In some embodiments, the kit comprises:

i) a forward primer comprising SEQ ID NO.: 3, a reverse primer comprising SEQ ID NO.: 4, and a probe comprising SEQ ID NO.: 5, for detecting the methylation state or level of at least one CpG dinucleotide of the BMP3 gene in the biological sample obtained from the patient, and
ii) a forward primer comprising SEQ ID NO.: 6, a reverse primer comprising SEQ ID NO.: 7, and a probe comprising SEQ ID NO.: 8, for detecting the methylation state or level of at least one CpG dinucleotide of the NDRG4 gene in the biological sample obtained from the patient.

In some embodiments, the kit comprises:

i) a forward primer comprising SEQ ID NO.: 9, a reverse primer comprising SEQ ID NO.: 10, and a probe comprising SEQ ID NO.: 11, for detecting the methylation state or level of at least one CpG dinucleotide of the BMP3 gene in the biological sample obtained from the patient, and
ii) a forward primer comprising SEQ ID NO.: 12, a reverse primer comprising SEQ ID NO.: 13, and a probe comprising SEQ ID NO.: 14, for detecting the methylation state or level of at least one CpG dinucleotide of the NDRG4 gene in the biological sample obtained from the patient.

In some embodiments, the kit comprises:

i) a forward primer comprising SEQ ID NO.: 15, a reverse primer comprising SEQ ID NO.: 16, and a probe comprising SEQ ID NO.: 17, for detecting the methylation state or level of at least one CpG dinucleotide of the BMP3 gene in the biological sample obtained from the patient, and
ii) a forward primer comprising SEQ ID NO.: 18, a reverse primer comprising SEQ ID NO.: 19, and a probe comprising SEQ ID NO.: 20, for detecting the methylation state or level of at least one CpG dinucleotide of the NDRG4 gene in the biological sample obtained from the patient.

In some embodiments, both the first probe and the second probe comprise a fluorescent donor and an acceptor fluorophore.

In some embodiments, the first probe and the second probe are TAQMAN® probes.

In some embodiments, the kit further comprises:

(1) means for detecting the presence or absence of at least one mutation in the KRAS gene in the patient; and
(2) means for detecting the presence or absence of hemoglobin in a biological sample obtained from the patient.

In some embodiments, the means for detecting the presence or absence of at least one mutation in the KRAS gene in the patient comprises at least one pair of primers capable of amplifying the Exon 12 and/or Exon 13 region of the KRAS gene in a polymerase chain reaction (PCR).

In some embodiments, the means for detecting the presence or absence of hemoglobin in the biological sample comprises an anti-hemoglobin antibody.

In some embodiments, the primers are capable of amplifying a KRAS gene region comprising at least one KRAS mutation selected from the group consisting G12D, G12V, G12C, G13D, G12A, G12R, G12S, and G13C.

In some embodiments, the antibody is a colloidal gold-conjugated antibody.

In some embodiments, the kit further comprises means for amplifying an internal quality control gene. An internal control can detect (1) inhibition contamination from sample or extraction method, (2) detect instrument malfunction, (3) chemistry failures (e.g., expired or degraded kit or components, or false combination of reagents), and (4) human error. In some embodiments, the internal control gene is a positive control, such as a gene in a positive control sample that has been determined to have methylation. In some embodiments, the internal control gene is a negative control, such as a gene in a negative control sample that has been determined not to have methylation.

In some embodiments, the kit further comprises instructions for use and/or interpretation of a test result obtained by using the kit.

In some embodiments, the kit further comprises means to detect a complex formed by the antibody and the hemoglobin in the biological sample.

In some embodiments, the biological sample obtained from the patient is a fecal sample.

In some embodiments, the kit further comprises a bisulfite reagent, and a container suitable for mixing the bisulfite reagent and the biological sample of the patient, or polynucleotides obtained from the biological sample.

In some embodiments, instead of using bisulfite, the kit further comprises a methylation sensitive restriction enzyme reagent.

In some embodiments, the kit further comprises: (1) a positive standard and a negative standard for detecting BMP3 methylation in the biological sample, and (2) a positive standard and a negative standard for detecting NDRG4 methylation in the biological sample.

In some embodiments, the positive standard for detecting BMP3 methylation comprises a polynucleotide sequence of:

(SEQ ID NO: 67)
GTTAGTTTGGTCGGGTGTTTTTAAAAATAAAGCGAGGAGGGAAGGTATA
GATAGATTTTGAAAATATTCGGGTTATATACGTCGCGATTTATAGTTTT
TTTTTAGCGTTGGAGTGGAGACGGCGTTCGTAGCGTTTTGCGCGGGTGA
GGTTCGCGTAGTTGTTGGGGAAGAGTTTATTTGTTAGGTTGCGTTGGGT
TAGCGTAGTAAGTGGGGTTGGTCGTTATTTCGTTGTATTCGGTCGCGTT
TCGGGTTTCGTGCGTTTTCGTTTTAG;

In some embodiments, the negative standard for detecting BMP3 methylation comprises a polynucleotide sequence of;

(SEQ ID NO: 68)
GTTAGTTTGGTTGGGTGTTTTTAAAAATAAAGTGAGGAGGGAACGTATAG
ATAGATTTTGAAAATATTTGGGTTATATATGTTGTGATTTATAGTTTTTT
TTTAGTGTTGGAGTGGAGATGGTGTTTGTAGTGTTTTGTGTGGGTGAGGT
TTGTGTAGTTGTTGGGGAAGAGTTTATTTGTTAGGTTGTGTTGGGTTAGT
GTAGTAAGTGGGGTTGGTTGTTATTTTGTTGTATTTGGTTGTGTTTTGGG
TTTTGTGTGTTTTTGTTTTAG;

In some embodiments, the positive standard for detecting NDRG4 methylation comprises a polynucleotide sequence of:

(SEQ ID NO.: 69)
TGAGAAGTCGGCGGGGGCGCGGATCGATCGGGGTGTTTTTTAGGTTTCGC
GTCGCGGTTTTCGTTCGTTTTTTCGTTCGTTTATCGGGTATTTTAGTCGC
GTAGAAGGCGGAAGTTACGCGCGAGGGATCGCGGTTCGTTCGGGATTAGT
TTTAGGTTCGGTATCGTTTCGCGGGTCGAGCGTTTATATTCGTTAAATTT
ACGCGGGTACGTTTTCGCGGCGTATCGTTTTTAGTT.

In some embodiments, the negative standard for detecting NDRG4 methylation comprises a polynucleotide sequence of:

(SEQ ID NO.: 70)
TGAGAAGTTGGTGGGGGTGTGGATTGATTGGGGTGTTTTTTAGGTTTTGT
GTTGTGGTTTTTGTTTGTTTTTTTGTTTGTTTATTGGGTATTTTAGTTGT
GTAGAAGGTGGAAGTTATGTGTGAGGGATTGTGGTTTGTTTGGGATTAGT
TTTAGGTTTGGTATTGTTTTGTGGGTTGAGTGTTTATATTTGTTAAATTT
ATGTGGGTATGTTTTTGTGGTGTATTGTTTTTAGTT.

Also provided is a method for detecting the presence or absence of colorectal cancer (CRC) or advanced adenoma (AA) in a patient in need thereof.

In some embodiments, the method comprises a) obtaining genomic DNA from a biological sample of the patient.

In some embodiments, the method further comprises b) treating the genomic DNA of a), or a fragment thereof, with one or more reagents to convert cytosine bases that are unmethylated thereof to uracil or another base that is detectably dissimilar to cytosine in terms of hybridization properties.

In some embodiments, the method further comprises c) contacting the treated genomic DNA, or the treated fragment thereof, with a first pair of primers for detecting the presence or absence of methylation sites of a gene encoding bone morphogenetic protein 3 (BMP3) in the patient. In some embodiments, the method further comprises contacting the treated genomic DNA, or a fragment thereof, with a second pair of primers for detecting the presence or absence of methylation sites of a gene encoding NDRG family member 4 protein (NDRG4) in the patient.

In some embodiments, the first pair of primers comprise a contiguous sequence of at least 9 nucleotides that is identical to, complementary to, or hybridizes under stringent hybridization conditions to SEQ ID NO.: 1. In some embodiments, the second pair of primers comprise a contiguous sequence of at least 9 nucleotides that is complementary to, or hybridizes under stringent hybridization conditions to SEQ ID NO.: 2.

In some embodiments, the treated genomic DNA or the fragment thereof is either amplified to produce at least one amplificate by the first pair of primers or the second pair of primers, or is not amplified.

In some embodiments, the method further comprises d) determining the presence or absence of CRC or AA in the patient, based on a presence or absence of said amplificate, the methylation state or level of at least one CpG dinucleotide of the BMP3 gene and the NDRG4 gene in the patient.

In some embodiments, a quantitative PCR is used to amplify the methylated BMP3 gene in the sample. In some embodiments, a quantitative PCR is used to amplify the methylated NDRG4 gene in the sample.

In some embodiments, the method also comprises using primers for amplifying a reference gene (a.k.a., normalizer, housekeeping gene, or endogenous control). In some embodiments, a quantitative PCR is used to amplify the reference gene in the sample.

In some embodiments, the first pair of primers and the first probe are selected from the group consisting of:

i) a forward primer comprising SEQ ID NO.: 3, a reverse primer comprising SEQ ID NO.: 4, and a probe comprising SEQ ID NO.: 5;
ii) a forward primer comprising SEQ ID NO.: 9, a reverse primer comprising SEQ ID NO.: 10, and a probe comprising SEQ ID NO.: 11; and
iii) a forward primer comprising SEQ ID NO.: 15, a reverse primer comprising SEQ ID NO.: 16, and a probe comprising SEQ ID NO.: 17.

In some embodiments, the second first pair of primers and the second probe are selected from the group consisting of:

iv) a forward primer comprising SEQ ID NO.: 6, a reverse primer comprising SEQ ID NO.: 7, and a probe comprising SEQ ID NO.: 8;
v) a forward primer comprising SEQ ID NO.: 12, a reverse primer comprising SEQ ID NO.: 13, and a probe comprising SEQ ID NO.: 14; and
vi) a forward primer comprising SEQ ID NO.: 18, a reverse primer comprising SEQ ID NO.: 19, and a probe comprising SEQ ID NO.: 20.

In some embodiments, the method comprises using

i) a forward primer comprising SEQ ID NO.: 3, a reverse primer comprising SEQ ID NO.: 4, and a probe comprising SEQ ID NO.: 5, for detecting the methylation state or level of at least one CpG dinucleotide of the BMP3 gene in the biological sample obtained from the patient, and
ii) a forward primer comprising SEQ ID NO.: 6, a reverse primer comprising SEQ ID NO.: 7, and a probe comprising SEQ ID NO.: 8, for detecting the methylation state or level of at least one CpG dinucleotide of the NDRG4 gene in the biological sample obtained from the patient.

In some embodiments, the method comprises using

i) a forward primer comprising SEQ ID NO.: 9, a reverse primer comprising SEQ ID NO.: 10, and a probe comprising SEQ ID NO.: 11, for detecting the methylation state or level of at least one CpG dinucleotide of the BMP3 gene in the biological sample obtained from the patient, and
ii) a forward primer comprising SEQ ID NO.: 12, a reverse primer comprising SEQ ID NO.: 13, and a probe comprising SEQ ID NO.: 14, for detecting the methylation state or level of at least one CpG dinucleotide of the NDRG4 gene in the biological sample obtained from the patient.

In some embodiments, the method comprises using

i) a forward primer comprising SEQ ID NO.: 15, a reverse primer comprising SEQ ID NO.: 16, and a probe comprising SEQ ID NO.: 17, for detecting the methylation state or level of at least one CpG dinucleotide of the BMP3 gene in the biological sample obtained from the patient, and
ii) a forward primer comprising SEQ ID NO.: 18, a reverse primer comprising SEQ ID NO.: 19, and a probe comprising SEQ ID NO.: 20, for detecting the methylation state or level of at least one CpG dinucleotide of the NDRG4 gene in the biological sample obtained from the patient.

In some embodiments, both the first probe and the second probe comprise a fluorescent donor and an acceptor fluorophore. In some embodiments, the first probe and the second probe are TAQMAN® probes.

In some embodiments, the method further comprises a step of detecting the presence or absence of at least one mutation in the KRAS gene in a biological sample obtained from the patient.

In some embodiments, the method further comprises a step of detecting the presence or absence of hemoglobin in a biological sample obtained from the patient. In some embodiments, the step of detecting the presence or absence of hemoglobin in the biological sample comprises using an anti-hemoglobin antibody. In some embodiments, the antibody is a colloidal gold-conjugated antibody.

In some embodiments, the step of detecting the presence or absence of at least one mutation in the KRAS gene in the patient comprises using at least one pair of primers capable of amplifying the Exon 12 and/or Exon 13 region of the KRAS gene in a polymerase chain reaction (PCR). In some embodiments, the primers are capable of amplifying a KRAS gene region comprising at least one KRAS mutation selected from the group consisting G12D, G12V, G12C, G13D, G12A, G12R, G12S, and G13C.

In some embodiments, the mutant KRAS gene is amplified by one or more pairs of primers selected from the group consisting of:

(1) a forward primer G12D-F comprising SEQ ID NO.: 35, and a reverse primer Kras-R comprising SEQ ID NO.: 42;
(2) a forward primer G13D-F comprising SEQ ID NO.: 36, and a reverse primer Kras-R comprising SEQ ID NO.: 42;
(3) a forward primer G12V-F comprising SEQ ID NO.: 37, and a reverse primer Kras-R comprising SEQ ID NO.: 42;
(4) a forward primer G12C-F comprising SEQ ID NO.: 38, and a reverse primer Kras-R comprising SEQ ID NO.: 42;
(5) a forward primer G12S-F comprising SEQ ID NO.: 39, and a reverse primer Kras-R comprising SEQ ID NO.: 42;
(6) a forward primer G12A-F comprising SEQ ID NO.: 40, and a reverse primer Kras-R comprising SEQ ID NO.: 42; and
(7) a forward primer G12R-F comprising SEQ ID NO.: 41, and a reverse primer Kras-R comprising SEQ ID NO.: 42.

In some embodiments, the KRAS probe for the qPCR comprises SEQ ID NO.: 46.

In some embodiments, the amplification of BMP3 gene is performed in a quantitative PCR (qPCR), and the method further comprises amplifying a first reference gene (i.e., a first reference gene) to determining the Ct value of the BMP3 amplification as ΔCt1.

In some embodiments, the amplification of NDRG4 gene is performed in a quantitative PCR (qPCR), and the method further comprises amplifying a second reference gene (i.e., a second reference gene) to determining the Ct value of the NDRG4 amplification as ΔCt2.

In some embodiments, the amplification of mutant KRAS gene is performed in a quantitative PCR (qPCR), and the method further comprises amplifying a third reference gene (i.e., a third reference gene) to determining the Ct value of the mutant KRAS amplification as ΔCt3.

In some embodiments, the first and the second reference genes are the same. In some embodiments, the same reference gene is a B2M gene.

In some embodiments, the third reference gene is an ACTB gene. In some embodiments, qPCR primers for amplifying ACTB gene comprise SEQ ID NOs.: 43 and 44, and the probe comprise SEQ ID NO.: 46.

In some embodiments, the method comprises using (1) a positive standard and a negative standard for detecting BMP3 methylation in the sample, and (2) a positive standard and a negative standard for detecting NDRG4 methylation in the sample.

In some embodiments, the positive standard for detecting BMP3 methylation comprises a polynucleotide sequence of.

(SEQ ID NO: 67)
GTTAGTTTGGTCGGGTGTTTTTAAAAATAAAGCGAGGAGGGAAGGTATA
GATAGATTTTGAAAATATTCGGGTTATATACGTCGCGATTTATAGTTTT
TTTTTAGCGTTGGAGTGGAGACGGCGTTCGTAGCGTTTTGCGCGGGTGA
GGTTCGCGTAGTTGTTGGGGAAGAGTTTATTTGTTAGGTTGCGTTGGGT
TAGCGTAGTAAGTGGGGTTGGTCGTTATTTCGTTGTATTCGGTCGCGTT
TCGGGTTTCGTGCGTTTTCGTTTTAG;

In some embodiments, the negative standard for detecting BMP3 methylation comprises a polynucleotide sequence of

(SEQ ID NO: 68)
GTTAGTTTGGTTGGGTGTTTTTAAAAATAAAGTGAGGAGGGAACGTATAG
ATAGATTTTGAAAATATTTGGGTTATATATGTTGTGATTTATAGTTTTTT
TTTAGTGTTGGAGTGGAGATGGTGTTTGTAGTGTTTTGTGTGGGTGAGGT
TTGTGTAGTTGTTGGGGAAGAGTTTATTTGTTAGGTTGTGTTGGGTTAGT
GTAGTAAGTGGGGTTGGTTGTTATTTTGTTGTATTTGGTTGTGTTTTGGG
TTTTGTGTGTTTTTGTTTTAG;

In some embodiments, the positive standard for detecting NDRG4 methylation comprises a polynucleotide sequence of

(SEQ ID NO.: 69)
TGAGAAGTCGGCGGGGGCGCGGATCGATCGGGGTGTTTTTTAGGTTTCGCG
TCGCGGTTTTCGTTCGTTTTTTCGTTCGTTTATCGGGTATTTTAGTCGCGT
AGAAGGCGGAAGTTACGCGCGAGGGATCGCGGTTCGTTCGGGATTAGTTTT
AGGTTCGGTATCGTTTCGCGGGTCGAGCGTTTATATTCGTTAAATTTACGC
GGGTACGTTTTCGCGGCGTATCGTTTTTAGTT.

In some embodiments, the negative standard for detecting NDRG4 methylation comprises a polynucleotide sequence of

(SEQ ID NO.: 70)
TGAGAAGTTGGTGGGGGTGTGGATTGATTGGGGTGTTTTTTAGGTTTTGTG
TTGTGGTTTTTGTTTGTTTTTTTGTTTGTTTATTGGGTATTTTAGTTGTGT
AGAAGGTGGAAGTTATGTGTGAGGGATTGTGGTTTGTTTGGGATTAGTTTT
AGGTTTGGTATTGTTTTGTGGGTTGAGTGTTTATATTTGTTAAATTTATGT
GGGTATGTTTTTGTGGTGTATTGTTTTTAGTT.

In some embodiments, the method comprises amplifying a quality control standard.

In some embodiments, a method for detecting the presence or absence of colorectal cancer (CRC) or advanced adenoma (AA) in a patient in need thereof, comprising using a kit of the present disclosure.

The present disclosure further provide a method for detecting the presence or absence of colorectal cancer (CRC) or advanced adenoma (AA) in a patient in need thereof, comprising:

a) obtaining an untreated genomic DNA from a fecal sample of the patient;
b) treating the genomic DNA of a), or a fragment thereof, with one or more reagents to convert cytosine bases that are unmethylated thereof to uracil or another base that is detectably dissimilar to cytosine in terms of hybridization properties;
c) performing a quantitative PCR (qPCR) using the treated genomic DNA of b) as a template, and determining the Ct value of BMP3 gene in the patient as ΔCt1;
d) performing a qPCR using the treated genomic DNA of b) as a template, and determining the Ct value of NDRG4 gene in the patient as ΔCt2;
e) performing a qPCR using the untreated genomic DNA as a template, and determining the Ct value of a mutant KRAS gene in the patient as ΔCt3;
f) performing a fecal immunochemical test of hemoglobin protein in the fecal sample and determining a score as FIT;
g) determining the value of K, wherein K=a*ΔCt1+b*ΔCt2+c*ΔCt3+d*FIT+X, wherein a, b, c, d, X are clinical constants; and
h) determining the value of a comprehensive index P, wherein P=e^{K}/(1+e^{K}), wherein e is the natural constant.

The clinical constants a, b, c, d, and X can be determined by analyzing clinical data distribution among a patient population.

In some embodiments, when P is equal or more than a predetermined threshold value, the patient is determined to have CRC and/or AA, and when P is less than the predetermined threshold value, the patient is determined to be health.

In some embodiments, the predetermined threshold value is calculated from clinical data distribution, such as clinical data obtained from patients that have been determined to have CRC and/or AA, and patients that have been determined not to have CRC and/or AA.

In some embodiments, the qPCR for amplifying BMP3 gene comprises a first pair of primers and a first probe, wherein the first pair of primers and the first probe are selected from the group consisting of:

i) a forward primer comprising SEQ ID NO.: 3, a reverse primer comprising SEQ ID NO.: 4, and a probe comprising SEQ ID NO.: 5;
ii) a forward primer comprising SEQ ID NO.: 9, a reverse primer comprising SEQ ID NO.: 10, and a probe comprising SEQ ID NO.: 11; and
iii) a forward primer comprising SEQ ID NO.: 15, a reverse primer comprising SEQ ID NO.: 16, and a probe comprising SEQ ID NO.: 17.

In some embodiments, the qPCR for amplifying NDRG4 gene comprises a second pair of primers and a second probe, wherein the second pair of primers and the second probe are selected from the group consisting of;

iv) a forward primer comprising SEQ ID NO.: 6, a reverse primer comprising SEQ ID NO.: 7, and a probe comprising SEQ ID NO.: 8;
v) a forward primer comprising SEQ ID NO.: 12, a reverse primer comprising SEQ ID NO.: 13, and a probe comprising SEQ ID NO.: 14; and
vi) a forward primer comprising SEQ ID NO.: 18, a reverse primer comprising SEQ ID NO.: 19, and a probe comprising SEQ ID NO.: 20.

In some embodiments, the method comprises using

i) a forward primer comprising SEQ ID NO.: 3, a reverse primer comprising SEQ ID NO.: 4, and a probe comprising SEQ ID NO.: 5, for detecting the methylation state or level of at least one CpG dinucleotide of the BMP3 gene in the sample, and
ii) a forward primer comprising SEQ ID NO.: 6, a reverse primer comprising SEQ ID NO.: 7, and a probe comprising SEQ ID NO.: 8, for detecting the methylation state or level of at least one CpG dinucleotide of the NDRG4 gene in the sample.

In some embodiments, the method comprises using

i) a forward primer comprising SEQ ID NO.: 9, a reverse primer comprising SEQ ID NO.: 10, and a probe comprising SEQ ID NO.: 11, for detecting the methylation state or level of at least one CpG dinucleotide of the BMP3 gene in the sample, and
ii) a forward primer comprising SEQ ID NO.: 12, a reverse primer comprising SEQ ID NO.: 13, and a probe comprising SEQ ID NO.: 14, for detecting the methylation state or level of at least one CpG dinucleotide of the NDRG4 gene in the sample.

In some embodiments, the method comprises using

i) a forward primer comprising SEQ ID NO.: 15, a reverse primer comprising SEQ ID NO.: 16, and a probe comprising SEQ ID NO.: 17, for detecting the methylation state or level of at least one CpG dinucleotide of the BMP3 gene in the sample, and
ii) a forward primer comprising SEQ ID NO.: 18, a reverse primer comprising SEQ ID NO.: 19, and a probe comprising SEQ ID NO.: 20, for detecting the methylation state or level of at least one CpG dinucleotide of the NDRG4 gene in the sample.

In some embodiments, both the first probe and the second probe comprise a fluorescent donor and an acceptor fluorophore. In some embodiments, the first probe and the second probe are TAQMAN® probes.

In some embodiments, the mutant KRAS gene comprising at least one KRAS mutation selected from the group consisting G12D, G12V, G12C, G13D, G12A, G12R, G12S, and G13C.

In some embodiments, the fecal immunochemical test comprises a colloidal gold-conjugated antibody.

In some embodiments, step c) and step d) of the method comprises using B2M gene as a reference gene.

In some embodiments, the method comprises using

(1) a positive standard and a negative standard for detecting BMP3 methylation in the sample, and
(2) a positive standard and a negative standard for detecting NDRG4 methylation in the sample.

In some embodiments, the positive standard for detecting BMP3 methylation comprises a polynucleotide sequence of

(SEQ ID NO: 67)
GTTAGTTTGGTCGGGTGTTTTTAAAAATAAAGCGAGGAGGGAAGGTATAGA
TAGATTTTGAAAATATTCGGGTTATATACGTCGCGATTTATAGTTTTTTTT
TAGCGTTGGAGTGGAGACGGCGTTCGTAGCGTTTTGCGCGGGTGAGGTTCG
CGTAGTTGTTGGGGAAGAGTTTATTTGTTAGGTTGCGTTGGGTTAGCGTAG
TAAGTGGGGTTGGTCGTTATTTCGTTGTATTCGGTCGCGTTTCGGGTTTCG
TGCGTTTTCGTTTTAG.

In some embodiments, the negative standard for detecting BMP3 methylation comprises a polynucleotide sequence of

(SEQ ID NO: 68)
GTTAGTTTGGTTGGGTGTTTTTAAAAATAAAGTGAGGAGGGAAGGTATAGA
TAGATTTTGAAAATATTTGGGTTATATATGTTGTGATTTATAGTTTTTTTT
TAGTGTTGGAGTGGAGATGGTGTTTGTAGTGTTTTGTGTGGGTGAGGTTTG
TGTAGTTGTTGGGGAAGAGTTTATTTGTTAGGTTGTGTTGGGTTAGTGTAG
TAAGTGGGGTTGGTTGTTATTTTGTTGTATTTGGTTGTGTTTTGGGTTTTG
TGTGTTTTTGTTTTAG.

In some embodiments, the positive standard for detecting NDRG4 methylation comprises a polynucleotide sequence of

(SEQ ID NO.: 69)
TGAGAAGTCGGCGGGGGCGCGGATCGATCGGGGTGTTTTTTAGGTTTCGCG
TCGCGGTTTTCGTTCGTTTTTTCGTTCGTTTATCGGGTATTTTAGTCGCGT
AGAAGGCGGAAGTTACGCGCGAGGGATCGCGGTTCGTTCGGGATTAGTTTT
AGGTTCGGTATCGTTTCGCGGGTCGAGCGTTTATATTCGTTAAATTTACGC
GGGTACGTTTTCGCGGCGTATCGTTTTTAGTT.

In some embodiments, the negative standard for detecting NDRG4 methylation comprises a polynucleotide sequence of

(SEQ ID NO.: 70)
TGAGAAGTTGGTGGGGGTGTGGATTGATTGGGGTGTTTTTTAGGTTTTGTG
TTGTGGTTTTTGTTTGTTTTTTTGTTTGTTTATTGGGTATTTTAGTTGTGT
AGAAGGTGGAAGTTATGTGTGAGGGATTGTGGTTTGTTTGGGATTAGTTTT
AGGTTTGGTATTGTTTTGTGGGTTGAGTGTTTATATTTGTTAAATTTATGT
GGGTATGTTTTTGTGGTGTATTGTTTTTAGTT.

In some embodiments, the method comprises amplifying a quality control standard in the step c) and the step d).

Also provided is a method for diagnosing and treating a colorectal cancer (CRC) and/or advanced adenoma (AA) in a patient in need thereof, comprising determining the presence or absence of CRC and/or AA in the patient by using a kit of the present disclosure, and treating the patient depends on the presence or absence of CRC and/or AA in the patient.

Also provided is a method for diagnosing and treating a colorectal cancer (CRC) and/or advanced adenoma (AA) in a patient in need thereof, comprising determining the presence or absence of CRC and/or AA in the patient by using a method described herein, and treating the patient depends on the presence or absence of CRC and/or AA in the patient.

### BRIEF DESCRIPTION OF THE DRAWINGS

FIG. 1A to FIG. 1D depict the results of CpG island prediction and relative position of amplicons of two BMP3 and NDRG4 genes. “Y”, “R” is degenerate bases. FIG. 1A—The result of CpG islands prediction of promoter region of BMP3 genes; FIG. 1B—The relative position of amplicons of BMP3 gene; FIG. 1C—The result of CpG islands prediction of promoter region of NDRG4 genes; FIG. 1D—The relative position of amplicons of NDRG4 gene.

FIG. 2A depicts the difference in methylation CpG sites of BMP in the white and Asian populations. FIG. 2B depicts the difference in methylation CpG sites of NDRG4 gene in the white and Asian populations.

FIG. 3A depicts the analytical sensitivity amplification curve of BMP3 with the primers and probes in the preferred group 1. FIG. 3B depicts the analytical sensitivity amplification curve of NDRG4 with primers and probes in the preferred group 1. FIG. 3C depicts the analytical sensitivity amplification curve of BMP3 with the primers and probes in the preferred group 2. FIG. 3D depicts the analytical sensitivity amplification curve of NDRG4 with primers and probes in the preferred group 2. FIG. 3E depicts the analytical sensitivity amplification curve of BMP3 with the primers and probes in the preferred group 3. FIG. 3F depicts the analytical sensitivity amplification curve of NDRG4 with primers and probes in the preferred group 3. FIG. 3G depicts the analytical sensitivity amplification curve of BMP3 with the primers and probes in the comparative group 1. FIG. 3H depicts the analytical sensitivity amplification curve of NDRG4 with primers and probes in the comparative group 1. FIG. 3I depicts the analytical sensitivity amplification curve of BMP3 with the primers and probes in the comparative group 2. FIG. 3J depicts the analytical sensitivity amplification curve of NDRG4 with primers and probes in the comparative group 2. FIG. 3K depicts the analytical sensitivity amplification curve of BMP3 with the primers and probes in the comparative group 3. FIG. 3L depicts the analytical sensitivity amplification curve of NDRG4 with primers and probes in the comparative group 3.

FIG. 4A depicts the analytical specificity amplification curve of BMP3 with the primers and probes in the preferred group 1. FIG. 4B depicts the analytical specificity amplification curve of NDRG4 with primers and probes in the preferred group 1. FIG. 4C depicts the analytical specificity amplification curve of BMP3 with the primers and probes in the preferred group 2. FIG. 4D depicts the analytical specificity amplification curve of NDRG4 with primers and probes in the preferred group 2. FIG. 4E depicts the analytical specificity amplification curve of BMP3 with the primers and probes in the preferred group 3. FIG. 4F depicts the analytical specificity amplification curve of NDRG4 with primers and probes in the preferred group 3. FIG. 4G depicts the analytical specificity amplification curve of BMP3 with the primers and probes in the comparative group 1. FIG. 4H depicts the analytical specificity amplification curve of NDRG4 with primers and probes in the comparative group 1. FIG. 4I depicts the analytical specificity amplification curve of BMP3 with the primers and probes in the comparative group 2. FIG. 4J depicts the analytical specificity amplification curve of NDRG4 with primers and probes in the comparative group 2. FIG. 4K depicts the analytical specificity amplification curve of BMP3 with the primers and probes in the comparative group 3. FIG. 4L depicts the analytical specificity amplification curve of NDRG4 with primers and probes in the comparative group 3.

FIG. 5A to FIG. 5C depicts the amplification curves using the primers and probes in the preferred group 1, preferred group 2, and preferred group 3 respectively for detecting BMP3 methylations in clinical sample. FIG. 5D to FIG. 5F depict the amplification curves using the primers and probes in the comparative group 1, comparative group 2, and comparative group 3 respectively for detecting BMP3 methylations in the same assay.

FIG. 6A to FIG. 6C depicts the amplification curves using the primers and probes in the preferred group 1, preferred group 2, and preferred group 3 respectively for detecting NDRG4 methylations in clinical sample. FIG. 6D to FIG. 6F depict the amplification curves using the primers and probes in the comparative group 1, comparative group 2, and comparative group 3 respectively for detecting NDRG4 methylations in the same assay.

### DETAIL DESCRIPTION

### Definitions

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Nucleic acid” or “oligonucleotide” or “polynucleotide”, as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

As used herein the phrase “subject in need thereof” refers to an animal or human subject who is known to have cancer, at risk of having cancer (e.g., a genetically predisposed subject, a subject with medical and/or family history of cancer, a subject who has been exposed to carcinogens, occupational hazard, environmental hazard) and/or a subject who exhibits suspicious clinical signs of cancer (e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness). Additionally or alternatively, the subject in need thereof can be a healthy human subject undergoing a routine well-being check up.

As used herein the term “about” refers to 10%.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5-SSC, and 1% SDS, incubating at 42° C., or, 5×SSC, 1% SDS, incubating at 65° C., with wash in 0.2×SSC, and 0.1% SDS at 65° C.

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

As used herein the term “diagnosing” refers to classifying pathology, or a symptom, determining a severity of the pathology (e.g., grade or stage), monitoring pathology progression, forecasting an outcome of pathology and/or prospects of recovery.

The phrase “consisting essentially of” means that the composition or method may include additional ingredients and/or steps, but only if the additional ingredients and/or steps do not materially alter the basic and novel characteristics of the claimed composition or method.

### 1. DNA Sequences Comprising Hypermethylated CpG Sites in the Promoter Region of BMP3 and NDRG4 Genes in Chinese Population of CRC and AA

This invention provides DNA sequences comprising of detailed hypermethylated CpG sites in the promoter region of BMP3 and NDRG4 genes in Asian population (e.g., the Chinese population), which is can be used as marker for CRC and AA detection.

In some embodiments, provided is the natural sequence of BMP3 gene as follows (5′ to 3′), showing potentially methylated sites marked by the superscript “m”:

(SEQ ID NO.: 65)
GCCAGTTTGGC^{m}CGGGTGTTCCCAAAAATAAAG^{m}CGAGGAGGGAAGGTACA
GACAGATCTTGAAAACACC^{m}CGGGCCACACA^{m}CGC^{m}CG^{m}CGACCTACAGCT
CTTTCTCAG^{m}CGTTGGAGTGGAGA^{m}CGG^{m}CGCCCGCAG^{m}CGCCCTG^{m}CG^{m}C
GGGTGAGGTC^{m}CG^{m}CGCAGCTGCTGGGGAAGAGCCCACCTGTCAGGCTG^{m}C
GCTGGGTCAG^{m}CGCAGCAAGTGGGGCTGGC^{m}CGCTATCT^{m}CGCTGCACCCG
GC^{m}CG^{m}CGTCC^{m}CGGGCTC^{m}CGTG^{m}CGCCCT^{m}CGCCCCAG.

In some embodiments, provided is the natural sequence of NDRG4 gene as follows (5′ to 3′), showing potentially methylated sites marked by the superscript “m”:

(SEQ ID NO.: 66)
TGAGAAGT^{m}CGG^{m}CGGGGG^{m}CG^{m}CGGAT^{m}CGAC^{m}CGGGGTGTCCCCCAGGC
TC^{m}CG^{m}CGT^{m}CG^{m}CGGTCCC^{m}CGCT^{m}CGCCCTCC^{m}CGCC^{m}CGCCCAC^{m}CGG
GCACCCCAGC^{m}CG^{m}CGCAGAAGG^{m}CGGAAGCCA^{m}CG^{m}CG^{m}CGAGGGAC^{m}C
G^{m}CGGTC^{m}CGTC^{m}CGGGACTAGCCCCAGGCC^{m}CGGCAC^{m}CGCCC^{m}CG^{m}CGG
GC^{m}CGAG^{m}CGCCCACACC^{m}CGCCAAACCCA^{m}CG^{m}CGGGCA^{m}CGCCCC^{m}CG^{m}
CGG^{m}CGCAC^{m}CGCCCCCAGCC.

After treating the natural genomic DNA, or a fragment thereof, with one or more reagents to convert cytosine bases that are unmethylated thereof to uracil (e.g., by bisulfite) or another base that is detectably dissimilar to cytosine in terms of hybridization properties, the converted sequence of BMP3 gene is as follow (5′ to 3′), showing potentially methylated sites marked by the superscript “m”:

(SEQ ID NO.: 1)
GTTAGTTTGGT^{m}CGGGTGTTTTTAAAAATAAAG^{m}CGAGGAGGGAAGGTATA
GATAGATTTTGAAAATATT^{m}CGGGTTATATA^{m}CGT^{m}CG^{m}CGATTTATAGTT
TTTTTTTAG^{m}CGTTGGAGTGGAGA^{m}CGG^{m}CGTT^{m}CGTAG^{m}CGTTTTG^{m}CG^{m}
CGGGTGAGGTT^{m}CG^{m}CGTAGTTGTTGGGGAAGAGTTTATTTGTTAGGTTG^{m}
CGTTGGGTTAG^{m}CGTAGTAAGTGGGGTTGGT^{m}CGTTATTT^{m}CGTTGTATT^{m}
CGGT^{m}CG^{m}CGTTT^{m}CGGGTTT^{m}CGTG^{m}CGTTTT^{m}CGTTTTAG.

After treating the natural genomic DNA, or a fragment thereof, with one or more reagents to convert cytosine bases that are unmethylated thereof to uracil (e.g., by bisulfite) or another base that is detectably dissimilar to cytosine in terms of hybridization properties, the converted sequence of NDRG4 gene is as follow (5′ to 3′), showing potentially methylated sites marked by the superscript “m”:

(SEQ ID NO.: 2)
TGAGAAGT^{m}CGG^{m}CGGGGG^{m}CG^{m}CGGAT^{m}CGAT^{m}CGGGGTGTTTTTTAGGT
TT^{m}CG^{m}CGT^{m}CG^{m}CGGTTTT^{m}CGTT^{m}CGTTTTTT^{m}CGTT^{m}CGTTTAT^{m}CGG
GTATTTTAGT^{m}CG^{m}CGTAGAAGG^{m}CGGAAGTTA^{m}CG^{m}CG^{m}CGAGGGAT^{m}C
G^{m}CGGTT^{m}CGTT^{m}CGGGATTAGTTTTAGGTT^{m}CGGTAT^{m}CGTTT^{m}CG^{m}CGG
GT^{m}CGAG^{m}CGTTTATATT^{m}CGTTAAATTTA^{m}CG^{m}CGGGTA^{m}CGTTTT^{m}CG^{m}
CGG^{m}CGTAT^{m}CGTTTTTAGTT.

The DNA sequences comprising of detailed hypermethylated CpG sites in the promoter region of BMP3 and NDRG4 genes in Asian population of the present disclosure is particularly useful for detecting CRC and/or AA in Asian population. For example, primers and probes can be designed to target one or more specific methylation sites in BMP3 and/or NDRG4 genes, as tools to determine the BMP3 and/or NDRG4 methylation state and level, therefore to determine the tumor condition in a patient in need thereof.

### 2. The Three Pairs of Preferred Primers and Probes for Detecting Methylation of BMP3 and NDRG4 Gene Respectively and Corresponding Reagents.

This invention provides three pairs of preferred primers and probes for detecting methylation levels of BMP3 and NDRG4 genes, respectively. These primers and probes are designed to target the high-frequency methylated CpG sites in the Asian population (e.g., the Chinese population).

These specific pairs of preferred primers and probes have surprisingly higher sensitivity and specificity in detecting CRC and AA, especially for AA detection in the Asian population, when compared to those in existing commercial products such as Cologuard®.

The sequences of primers and probes are as follows:

| Gene | Group | Primer/Probe | Sequence ID | Sequences (5′ to 3′) |
|---|---|---|---|---|
| BMP3 | Preferred 1 | Forward Primer | SEQ ID NO.: 3 | TTTGAAAATATTCGGGTTATATACGTCGC |
| | | Reverse Primer | SEQ ID NO.: 4 | ATAAACTCTTCCCCAACAACTACGCGAA |
| | | Probe | SEQ ID NO.: 5 | AGCGTTGGAGTGGAGACGGCGTTCG |
| | Preferred 2 | Forward Primer | SEQ ID NO.: 9 | AATATTCGGGTTATATACGTCGCGA |
| | | Reverse Primer | SEQ ID NO.: 10 | GCAACCTAACAAATAAACTCTTCCCCAA |
| | | Probe | SEQ ID NO.: 11 | TGGAGTGGAGACGGCGTTCGTAGCGT |
| | Preferred 3 | Forward Primer | SEQ ID NO.: 15 | AATATTCGGGTTATATACGTCGCGATT |
| | | Reverse Primer | SEQ ID NO.: 16 | ACTTACTACGCTAACCCAACG |
| | | Probe | SEQ ID NO.: 17 | TAGCGTTGGAGTGGAGACGGCGTTCGTA |
| NDRG4 | Preferred 1 | Forward Primer | SEQ ID NO.: 6 | ATCGATCGGGGTGTTTTTTAGGTTTC |
| | | Reverse Primer | SEQ ID NO.: 7 | CCTTCTACGCGACTAAAATACCCGAT |
| | | Probe | SEQ ID NO.: 8 | CGTCGCGGTTTTCGTTCGTTTTTTCGTTCGT |
| | Preferred 2 | Forward Primer | SEQ ID NO.: 12 | GCGGGTGAGAAGTCGGC |
| | | Reverse Primer | SEQ ID NO.: 13 | GTAACTTCCGCCTTCTACGC |
| | | Probe | SEQ ID NO.: 14 | TAGGTTTCGCGTCGCGGTTTTCGTT |
| | Preferred 3 | Forward Primer | SEQ ID NO.: 18 | CGGTTTTCGTTCGTTTTTTCG |
| | | Reverse Primer | SEQ ID NO.: 19 | AACCTAAAACTAATCCCGAACGAACC |
| | | Probe | SEQ ID NO.: 20 | TCGTTTATCGGGTATTTTAGTCGCGTAG |

The oligonucleotides of the present disclosure, advantageously permit the extremely specific amplification of the hypermethylated CpG sites in the promoter region of BMP3 or NDRG4 in a biological sample obtained from an Asian patient.

In some embodiments, provided are oligonucleotides that are partially or completely complementary to a sequence of SEQ ID NOs: 3 to 20.

In some embodiments, provided are oligonucleotides having one or more modifications compared to a probe sequence, such as SEQ ID NOs: 5, 11, 17, 8, 14, and 20. In some embodiments, the modification can happen at the 5′ end and/or 3′ end of one of the nucleotide sequences recited in SEQ ID NO.: 5, 11, 17, 8, 14, and 20.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine amongst others.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In some embodiments, an oligonucleotide in a sequence of SEQ ID NOs: 5, 11, 17, 8, 14, and 20 is replaced by a unnatural nucleotide, such as an artificial nucleic acid. Artificial nucleic acids include, but are not limited to, peptide nucleic acid (PNA), Morpholino, locked nucleic acid (LNA), glycol nucleic acid (GNA) and threose-nucleic acid (TNA). Each of these is distinguished from naturally occurring DNA or RNA by changes to the backbone of the molecule.

In some embodiments, a probe of the present disclosure comprises a label at the 5′ and the probe.

In some embodiments, the label at the 5′ of a probe comprises a fluorescent dye, such as a fluorophore. As used herein, fluorophore is a fluorescent chemical compound that can re-emit light upon light excitation. Fluorophores typically contain several combined aromatic groups, or planar or cyclic molecules with several n bonds. Non-protein organic fluorophores include, but are not limited to, xanthene derivative (e.g., fluorescein, rhodamine, Oregon green, eosin, and Texas red); cyanine derivatives (e.g., cyanine, indocarbocyanine, oxacarbocyanine, thiacarbocyanine, and merocyanine), squaraine derivatives and ring-substituted squaraines (e.g., Seta, SeTau, and Square dyes), naphthalene derivatives (e.g., dansyl and prodan derivatives), coumarin derivatives; oxadiazole derivatives (e.g., pyridyloxazole, nitrobenzoxadiazole and benzoxadiazole); anthracene derivatives (e.g., anthraquinones, including DRAQ5, DRAQ7 and CyTRAK Orange); pyrene derivatives (cascade blue, etc.), oxazine derivatives (e.g., Nile red, Nile blue, cresyl violet, oxazine 170, etc.; acridine derivatives (e.g., proflavin, acridine orange, acridine yellow, etc.); arylmethine derivatives (e.g., auramine, crystal violet, malachite green); tetrapyrrole derivatives (e.g., porphin, phthalocyanine, bilirubin). Particular examples include, but are not limited to, VIC, PET, Texas Red, Cy3, Cy5, FAM(6-Carboxyfluorescein), HEX (6-carboxy-2′,4,4′,5′,7,7′-hexachlorofluorescein), ROX (5(6)-Carboxy-X-rhodamine), JOE (6-carboxy-4′,5′-dichloro-21,71-dimethoxyfluorescein), TET (5′-tetrachloro-fluorescein phosphoramidite), NED (fluorescein benzoxanthene), TAMRA (6-carboxy-N,N,N,N-tetramethylrhodamine), FITC (fluorescein isothiocyanate). Examples of particular fluorophores that can be used in the probes disclosed herein are known to those of skill in the art and include those provided in U.S. Pat. No. 5,866,366 to Nazarenko et al., such as 4-acetamido-4′-isothiocyanatostilbene-2,2′disulfonic acid; acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), QFITC (XRITC), -6-carboxy-fluorescein (HEX), and TET (Tetramethyl fluorescein); fluorescamine; IR144; IR1446; Malachite Green isothiocyanate; 4-methylumbelliferone; ortho cresolphthalein; nitrotyrosine; pararosanilin; Phenol Red; B-phycoerythrin; o-phthaldialdehyde; pyrene and derivatives such as pyrene, pyrene butyrate and succinimidyl 1-pyrene butyrate; Reactive Red 4 (CIBACRON™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, N,N,N′,N′-tetramethyl-6-carboxyrhodamine (TAMRA), tetramethyl rhodamine, and tetramethyl rhodamine isothiocyanate (TRITC); sulforhodamine B; sulforhodamine 101 and sulfonyl chloride derivative of sulforhodamine 101 (Texas Red); riboflavin; rosolic acid and terbium chelate derivatives; LightCycler Red 640; Cy5.5; and Cy56-carboxyfluorescein; boron dipyrromethene difluoride (BODIPY); acridine; stilbene; 6-carboxy-X-rhodamine (ROX); Cy3; Cy3.5, Cy5, Cy5.5, VIC® (Applied Biosystems); LC Red 640; LC Red 705; OregonGreen™; CALRed™; Red640; and Yakima yellow; LighterCycler®Cyan500; LighterCycler®; Red610; Alexa 647; Alexa 555; 5-(2-aminoethyl)amino-1-naphthalene sulfonic acid (EDANS); tetramethyl rhodamine (TMR); tetramethyl rhodamine isocyanate (TMRITC), fluorescein isocyanate (FITC), χ-rhodamine, derivatives thereof, or any combination thereof, amongst others. More fluorescent dyes are described in U.S. Pat. Nos. 5,866,366, 6,818,431, 6,056,859, 9,140,688, 9,581,587, 6,165,765, 6,485,909, 8,158,358, 7,625,723, 7,560,236, 7,867,701, 9,150,912, 7,960,543, 6,555,383, 6,881,570, 8,198,026, 5,625,081, 8,445,291, 9,194,801, 8,835,110, 7,893,227, 9,243,289, 7,427,674, 9,512,493, US Patent Application Publication Nos: 20170152552, 20030170672, 20160281151, 20130084558, 20060281100, 20140234833, 20150072340, 20050089910, 20090081677, 2014002402220180171393, 20060188886, 20010018185, 20110151446, and WO/2000/017330A1, WO/2008/030071A1, WO/2013/049631A1, WO/2016/179090A1, WO/2016/123895A1, WO/2003/079022A1, each of which is herein incorporated by reference in its entirety.

In some embodiments, a probe of the present disclosure comprises a fluorescent donor and an acceptor fluorophore. As used herein, an acceptor fluorophore (e.g., a “fluorescent quencher”), is a fluorophore which absorbs energy from a donor fluorophore, for example in the range of about 400 to 900 nm. Acceptor fluorophores generally absorb light at a wavelength which is usually at least 10 nm higher (such as at least 20 nm higher) than the maximum absorbance wavelength of the donor fluorophore. Acceptor fluorophores have an excitation spectrum which overlaps with the emission of the donor fluorophore, such that energy emitted by the donor can excite the quencher. Any acceptor fluorophores known in the art can be utilized. In a particular example, an acceptor fluorophore is a dark quencher, such as Dabcyl, QSY7 (Molecular Probes), QSY9 (Molecular Probes), QSY21 (Molecular Probes), QSY33 (Molecular Probes), BLACK HOLE QUENCHERS™ (Glen Research, e.g., BHQ-1, BHQ-2, BHQ-3), ECLIPSE™ Dark Quencher (Epoch Biosciences), DDQ-I, DDQ-II, Dabcyl, Eclipse, or IOWA BLACK™ (Integrated DNA Technologies, e.g., Iowa Black FQ, Iowa Black RQ). More fluorescent quenchers are described in U.S. Pat. Nos. 9,957,546, 9,274,008, US Patent Publication Nos: 20140295422, 20090042205, 20160281182, 20180142284, 20140147929, and WO/2009/009615A1, WO/2016/160572A1, WO/2016/178953A1, WO/2018/229663A1, WO/2010/051544A2, WO/2013/152220A2, each of which is herein incorporated by reference in its entirety. A quencher can reduce or quench the emission of a donor fluorophore. In such an example, instead of detecting an increase in emission signal from the acceptor fluorophore when in sufficient proximity to the donor fluorophore (or detecting a decrease in emission signal from the acceptor fluorophore when a significant distance from the donor fluorophore), an increase in the emission signal from the donor fluorophore can be detected when the quencher is a significant distance from the donor fluorophore (or a decrease in emission signal from the donor fluorophore when in sufficient proximity to the quencher acceptor fluorophore).

In some embodiments, primers and probes of the present disclosure are based on fluorescence resonance energy transfer (FRET). Examples of oligonucleotides using FRET that can be used to detect amplicons include linear oligoprobes, such as HybProbes, 5′ nuclease oligoprobes, such as TAQMAN® probes, hairpin oligoprobes, such as molecular beacons, scorpion primers and UniPrimers, minor groove binding probes, and self-fluorescing amplicons, such as sunrise primers.

In some embodiments, primers and/or probes of the present disclosure are labeled by other functional entities, such as biotin, haptenes, antigens, chemical groups, radioactive substances, enzymatic markers, etc. The detection of a marked amplification product may be accomplished, for example, using fluorescence methods, chemoluminescence methods, densitometry methods, photometry methods, precipitation reactions, enzymatic reactions including enzymatic reinforcement reactions, SPR (“surface plamon resonance”) methods, ellipsometry methods, measurement of the index of refraction, measurement of reflectance, and similar methods.

In some embodiments, primers and probes described herein can be used in a quantitative PCR to determine the methylation state and level in BMP3 and/or NDRG4 gene in a patient. In some embodiments, an additional reaction can be included to amplify one or more reference gene. In some embodiments, the reference gene is a gene in a patient whose activity would not be affected by the presence or absence of CRC and AA, and would not be affected by the methylation state and level of BMP3 and NDRG4. In some embodiments, the reference genes include, but are not limited to β-globin (HBB), telomerase (TERT), glyceraldehyde-3-phosphate dehydrogenase (GAPDH), albumin (ALB), β-actin (ACTB), Beta 2 microglobulin (B2M), and T cell receptor γ (TRG).

In some embodiments, B2M gene is used as a reference gene in the quantitative PCR for detecting methylation state and level of BMP3/NDRG4.

In some embodiments, one or more other controls can be introduced, including but not limited to, no-template control (for detecting reagent or equipment contamination and to confirm positive results); no-amplification control (for detecting background fluorescence generated by degraded labelled probes), and positive control (for detecting inhibitors or malfunction, and to confirm that reagents and equipment are working).

In some embodiments, a qPCR is used for determining if there is amplification of methylated BMP3 gene or methylated NDRG4 gene in the sample. The detected signal from the probe of BMP3 or NDRG4 is quantitated either by reference to a standard curve or by comparing the Ct values to that of a reference gene. Analysis of housekeeping genesis often used to normalize the results. The cycle threshold (Ct) is defined as the number of cycles required for the fluorescent signal to cross a predetermined threshold (e.g., exceeding the background level, such as exceeding the level of amplification in a negative control sample). In some embodiments, the threshold is automatically determined by the software of the qPCR instrument or other suitable methods. In some embodiments, the threshold is set just above (e.g., about 0.01%, 0.1%, 1%, 5%, or 10% higher) the terminal fluorescent value in a negative control sample.

In some embodiments, when the Ct value associated with BMP3 or NDRG4 amplification in a test sample is no more than (≤) about 35, 34, 33, 32, 31, 30, or less, the sample is determined as containing methylated BMP3 or NDRG4, and the patient has CRC and/or AA (positive result), otherwise the sample is determined as not containing the methylated BMP3 or NDRG4, and the patient does not have CRC or AA (negative result). For the reference gene amplification, when the Ct value associated with a control gene amplification in the sample is not more than (≤) about 34, 33, 32, 31, 30, 29 or less, the reference gene amplification is determined to be positive, otherwise the reference gene amplification is determined to be negative. When the reference gene amplification is determined to be negative, the test result is invalidated.

In some embodiments, the difference between the Ct value associated with BMP3 is compared to the Ct value associated with the reference gene amplification (ΔCt=Ct_{gene of interest}−Ct_{reference gene}) and referred as ΔCt1. In some embodiments, when ΔCt1 is not more than a predetermined critical value (≤the critical value), then the sample is determined to have BMP3 methylation (positive result), and the patient is determined to have CRC or AA. In some embodiments, when ΔCt1 is more than a predetermined critical value (>the critical value), then the sample is determined not to have BMP3 methylation (negative result), and the patient is determined to be healthy. In some embodiments, the critical value is the corresponding ΔCt value for a sample comprising 5 ng/μL nucleotide sequence having a methylation rate of 1%, such as about 8, 9, or 10.

In some embodiments, the difference between the Ct value associated with NDRG4 is compared to the Ct value associated with the reference gene amplification (ΔCt=Ct_{gene of interest}−Ct_{reference gene}) and referred as ΔCt2. In some embodiments, when ΔCt2 is not more than a predetermined critical value (≤the critical value), then the sample is determined to have NDRG4 methylation (positive result), and the patient is determined to have CRC or AA. In some embodiments, when ΔCt2 is more than a predetermined critical value (>the critical value), then the sample is determined not to have NDRG4 methylation (negative result), and the patient is determined to be healthy. In some embodiments, the critical value is the corresponding ΔCt value for a sample comprising 5 ng/μL nucleotide sequence having a methylation rate of 1%, such as about 8, 9, or 10.

Preferred primers and probes of the present disclosure have surprisingly high sensitivity and specificity in detecting CRC and AA in the Asian population, especially for AA detection, when compared to those in existing commercial products such as Cologuard®.

As used herein, the term “sensitivity” refers to the rate when patients actually having CRC and/or AA in a given population are correctly detected. In some embodiments, the sensitivity is at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more, or 100%. In some embodiments, the size of the population is at least about 50, 100, 200, 300, 400, 500, 600, 700, 800, 900, 1000, 2000, 3000, 4000, 5000, 6000, 7000, 8000, 9000, 10,000, or more.

As used herein, the term “specificity” refers to the rate when patients actually not having CRC or AA a given population are correctly diagnosed as not having the condition. In some embodiments, the specificity is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more, or 100%. In some embodiments, the size of the population is at least about 50, 100, 200, 300, 400, 500, 600, 700, 800, 900, 1000, 2000, 3000, 4000, 5000, 6000, 7000, 8000, 9000, 10,000, or more.

As demonstrated in the Examples, the preferred primers and probes provide surprisingly high sensitivity and specificity in detecting methylation of BMP3 and NDRG4, therefore lead to surprisingly high sensitivity and specificity in detecting CRC and/or AA. For example, the sensitivity of CRC detection with the three pairs of preferred primers and probes of BMP3 gene is at least 85%; the sensitivity of CRC detection with the three pairs of preferred primers and probes of NDRG4 gene is at least 90%; the sensitivity of AA detection with the three pairs of preferred primers and probes of BMP3 gene is at least 66%; the sensitivity of AA detection with the three pairs of preferred primers and probes of NDRG4 gene is at least 73%. Also, the specificity of CRC and AA detection with the three pairs of preferred primers and probes of BMP3 gene is about 97%-100% (e.g., at least about 97.8%); the specificity of CRC and AA detection with the three pairs of preferred primers and probes of NDRG4 gene is also about 97%-100% (e.g., at least about 97.8%).

3. Three Sets of Preferred Primers-Probe Combinations for Detecting CRC and/or AA Inpatients.

The present disclosure also provides three sets of preferred primer and probe combinations for detecting methylation levels of BMP3 and NDRG4 genes, in order to determine the presence or absence of CRC or AA in a patient. These specific combinations have surprisingly higher sensitivity and specificity in detecting CRC and AA in the Asian population, especially for AA detection, when compared to those in existing commercial products such as Cologuard®

The sequences of three sets of preferred primers and probes are as follows:

| Combination | | | Primers/ | | |
|---|---|---|---|---|---|
| No. | Gene | Group | Probes | Sequence ID | Sequence (5′ to 3′) |
| 4 | BMP3 | Preferred 1 | Forward | SEQ ID NO.: 3 | TTTGAAAATATTCGGGTTATATACGTCGC |
| | | | Primer | | |
| | | | Reverse Primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred 1 | Forward | SEQ ID NO.: 6 | ATCGATCGGGGTGTTTTTTAGGTTTC |
| | | | Primer | | |
| | | | Reverse Primer | SEQ ID NO.: 7 | CCTTCTACGCGACTAAAATACCCGAT |
| | | | Probe | SEQ ID NO.: 8 | CGTCGCGGTTTTCGTTCGTTTTTTCGTTCGT |
| 5 | BMP3 | Preferred 1 | Forward | SEQ ID NO.: 3 | TTTGAAAATATTCGGGTTATATACGTCGC |
| | | | Primer | | |
| | | | Reverse Primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred 2 | Forward | SEQ ID NO.: 12 | GCGGGTGAGAAGTCGGC |
| | | | Primer | | |
| | | | Reverse Primer | SEQ ID NO.: 13 | GTAACTTCCGCCTTCTACGC |
| | | | Probe | SEQ ID NO.: 14 | TAGGTTTCGCGTCGCGGTTTTCGTT |
| 7 | BMP3 | Preferred 2 | Forward | SEQ ID NO.: 9 | AATATTCGGGTTATATACGTCGCGA |
| | | | Primer | | |
| | | | Reverse Primer | SEQ ID NO.: 10 | GCAACCTAACAAATAAACTCTTCCCCAA |
| | | | Probe | SEQ ID NO.: 11 | TGGAGTGGAGACGGCGTTCGTAGCGT |
| | NDRG4 | Preferred 1 | Forward | SEQ ID NO.: 6 | ATCGATCGGGGTGTTTTTTAGGTTTC |
| | | | Primer | | |
| | | | Reverse Primer | SEQ ID NO.: 7 | CCTTCTACGCGACTAAAATACCCGAT |
| | | | Probe | SEQ ID NO.: 8 | CGTCGCGGTTTTCGTTCGTTTTTTCGTTCGT |

As demonstrated in the Examples, the preferred combinations of BMP3 and NDRG4 primers-probe sets provide surprisingly high sensitivity and specificity in detecting methylation of BMP3 and NDRG4, therefore lead to surprisingly high sensitivity and specificity in detecting CRC and/or AA. In some embodiments, this sensitivity and specificity are obtained when the same assay also includes KRAS gene analysis and hemoglobin test, as explained in Example 5. For example, the overall sensitivity of CRC detection with the three preferred combinations of primers and probes of BMP3 and NDRG4 genes in combination of the KRAS gene analysis and the hemoglobin test is at least 95%; the sensitivity of AA detection with the three preferred combinations of primers and probes of BMP3 and NDRG4 gene in combination of the KRAS gene analysis and the hemoglobin test is at least 93%; the sensitivity of CRC+AA detection with the three preferred combinations of primers and probes of BMP3 and NDRG4 genes in combination of the KRAS gene analysis and the hemoglobin test is at least 97%; the specificity of CRC+AA detection with the three preferred combinations of primers and probes of BMP3 and NDRG4 genes in combination of the KRAS gene analysis and the hemoglobin test is at least 97%.

### 4. The Kit for Detecting CRC and AA

The present disclosure provides kits for BMP3/NDRG4 methylation detection, and/or for CRC/AA detection in a patient in need therefore. In some embodiments, the kits are particularly suitable for Asian patients, such as Chinese patients.

In some embodiments, this kit comprise: (1) at least one of the three sets of preferred primer and probe combinations for detecting CRC and AA (Combination Nos. 4, 5, and 7 in Table 20), and corresponding qPCR reagents; (2) means for detecting seven mutations in the coding region of KRAS gene (i.e., PG12 [symbol] G13D [symbol] G12V [symbol] G12C [symbol] G12S [symbol] G12A [symbol] and G13R), such as suitable primers and probes, and corresponding PCR reagents; (3) means for detecting hemoglobin in as tool sample, such as reagents based on FIT technology (e.g., an anti-hemoglobin antibody, and reagents for detecting the complex formed by the antibody and hemoglobin in the stool sample).

In some embodiments, the kit comprises at least one set of the preferred primer-probe combination below:

(1) The three sets of preferred primer and probe combinations is as follows:

| NO. | Gene | Group | Primer/Probe | Sequence ID | Sequence (5′ to 3′) |
|---|---|---|---|---|---|
| 4 | BMP3 | Preferred 1 | Forward Primer | SEQ ID NO.: 3 | TTTGAAAATATTCGGGTTATATACGTCGC |
| | | | Reverse Primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred 1 | Forward Primer | SEQ ID NO.: 6 | ATCGATCGGGGTGTTTTTTAGGTTTC |
| | | | Reverse Primer | SEQ ID NO.: 7 | CCTTCTACGCGACTAAAATACCCGAT |
| | | | Probe | SEQ ID NO.: 8 | CGTCGCGGTTTTCGTTCGTTTTTTCGTTCGT |
| 5 | BMP3 | Preferred 1 | Forward Primer | SEQ ID NO.: 3 | TTTGAAAATATTCGGGTTATATACGTCGC |
| | | | Reverse Primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred 2 | Forward Primer | SEQ ID NO.: 12 | GCGGGTGAGAAGTCGGC |
| | | | Reverse Primer | SEQ ID NO.: 13 | GTAACTTCCGCCTTCTACGC |
| | | | Probe | SEQ ID NO.: 14 | TAGGTTTCGCGTCGCGGTTTTCGTT |
| 7 | BMP3 | Preferred 2 | Forward Primer | SEQ ID NO.: 9 | AATATTCGGGTTATATACGTCGCGA |
| | | | Reverse Primer | SEQ ID NO.: 10 | GCAACCTAACAAATAAACTCTTCCCCAA |
| | | | Probe | SEQ ID NO.: 11 | TGGAGTGGAGACGGCGTTCGTAGCGT |
| | NDRG4 | Preferred 1 | Forward Primer | SEQ ID NO.: 6 | ATCGATCGGGGTGTTTTTTAGGTTTC |
| | | | Reverse Primer | SEQ ID NO.: 7 | CCTTCTACGCGACTAAAATACCCGAT |
| | | | Probe | SEQ ID NO.: 8 | CGTCGCGGTTTTCGTTCGTTTTTTCGTTCGT |

### (2) Triplex Quantitative PCR Reagents for Detecting Methylation Level of BMP3 and NDRG4 Genes.

In some embodiments, the kits comprise reagents to perform a multiplex PCR for detecting methylation of BMP3 and NDRG4 at the same time. In some embodiments, the multiplex PCR is a quantitative PCR.

In some embodiments, the reagents include Taq DNA polymerase. In some embodiments, the final concentration of Taq DNA polymerase is about 2 U/reaction. In some embodiments, the reagents include MgCl₂. In some embodiments, the final concentration of MgCl₂ in the reaction is 2 mM. In some embodiments, the reagents include dNTPs. In some embodiments, the dNTP has a final concentration of 0.2 mM. In some embodiments, the reagents include about 0.5 mM to 0.75 mM primers of amplifying BMP3, NDRG4 and reference genes. In some embodiments, the reagents include about 0.1 mM to 0.25 mM probes hybridized to DNA sequences of BMP3, NDRG4 and reference genes. In some embodiments, the reagents further include PCR buffer, such as concentrated PCR buffer (e.g., 5× or 10×), which can be diluted to the final concentration of 1×. In some embodiments, B2M is the reference gene and amplified for quality control in quantitative PCR.

### (3) Primers and Probes for Detecting Seven Mutations in the Coding Region of KRAS Gene.

In some embodiments, the kits comprise means for detecting mutations in the KRAS gene. In some embodiments, the kits comprise primers and probes that are designed to amplify and detect seven mutant hotspots in Exon 12 and Exon 13 in the open reading region of KRAS gene, which are G12D, G13D, G12V, G12C, G12S, G12A, and G13R. In some embodiments, the sequences of primers and probes are as follows:

| Name | Primer/Probe | Sequence ID | Sequences (5′ to 3′) |
|---|---|---|---|
| G12D-F | Forward primer | SEQ ID NO.: 35 | AACTTGTGGTAGTTGGAGGTGA |
| G13D-F | Forward primer | SEQ ID NO.: 36 | AACTTGTGGTAGTTGGAGCTGGGGA |
| G12V-F | Forward primer | SEQ ID NO.: 37 | AACTTGTGGTAGTTGGAGTTGT |
| G12C-F | Forward primer | SEQ ID NO.: 38 | AAACTTGTGGTAGTTGGGGCTT |
| G12S-F | Forward primer | SEQ ID NO.: 39 | AAACTTGTGGTAGTTGGTGCTA |
| G12A-F | Forward primer | SEQ ID NO.: 40 | AACTTGTGGTAGTTGGAGCAGC |
| G12R-F | Forward primer | SEQ ID NO.: 41 | AAACTTGTGGTAGTTGGAGCTC |
| Kras-R | Reverse primer | SEQ ID NO.: 42 | GAATGGTCCTGCACCAGTAATATG |
| ACTB-F | Forward primer | SEQ ID NO.: 43 | AGGGCTTCTTGTCCTTTCCTT |
| ACTB-R | Reverse primer | SEQ ID NO.: 44 | CGTGCTCGATGGGGTACTTC |
| KRAS-P | Probe | SEQ ID NO.: 45 | AGGCAAGAGTGCCTTGACGATACAGC |
| ACTB-P | Probe | SEQ ID NO.: 46 | CGTGATGGTGGGCATGGGTCAGAAGGA |

### (4) The Multiplex Quantitative PCR Reagents for Detecting Codon Mutations of KRAS Gene.

Also provided is a multiplex quantitative PCR system for detecting all seven mutations of KRAS gene. In some embodiments, a multiplex quantitative PCR reaction comprise Taq DNA polymerase with final concentration of 2.5 U/reaction, MgCl₂ with final concentration of 1 mM, dNTPs with final concentration of 0.1 mM, 0.3-0.9 μM primers of amplifying KRAS and ACTB genes, 0.05-0.1 μM probes hybridized to DNA sequences of KRAS and ACTB genes, and 1×PCR buffer. ACTB is a reference gene and amplified for quality control in quantitative PCR.

### (5) The Kit of Detecting Hemoglobin in Feces

In some embodiments, the kits comprise reagents for detecting hemoglobin. In some embodiments, hemoglobin is tested qualitatively by enzyme-linked immune sorbent assay (ELISA).

### 5. The Logistic Regression Model

In some embodiments, after having the results of the BMP3/NDRG4 methylation test, the KRAS mutation test, and the hemoglobin test, all results are compiled and subjected to a logistic regression model, in order to determine the presence or the absence of CRC and/or AA in the patients.

In some embodiments, the method comprises calculating the value of a comprehensive cancer index value P, wherein P=e^{K}/(1+e^{K}), wherein e is the natural constant.

In some embodiments, K is defined to be K=a*ΔCt1+b*ΔCt2+c*ΔCt3+d*FIT+X, wherein a, b, c, d, X are clinical constant. ΔCt, ΔCt2 and ΔCt3 are Ct value of BMP3, NDRG4 and KRAS subtract to that of reference genes.

In some embodiments, when P value is equal or more than predetermined threshold, the test result is positive, otherwise negative. Positive result indicates the person is possible to have CRC or AA, otherwise is healthy.

### 6. Treatment Methods

Methods of the present disclosure in some embodiments comprise treating the patients in need after the patients are classified to having colorectal cancer and/or adenoma. In some embodiments, the treating include, but are not limited to, surgery, chemotherapy, radiation therapy, immunotherapy, palliative care, exercise.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relieve symptoms of the pathology yet results in incomplete cure of the pathology. It will be appreciated that in certain cases the treatment regimen may be associated with some discomfort to the subject or adverse side effects (e.g., damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

In some embodiments, the treatments include, but is not limited to, fluorouracil, capecitabine, oxaliplatin, irinotecan, UFT, FOLFOX, FOLFOXIRI, and FOLFIRI, antiangiogenic drugs such as bevacizumab, and epidermal growth factor receptor inhibitors (e.g., cetuximab and panitumumab).

### 7. Advantages of the Present Invention

Without wishing to be bound by any particular theory, the present invention at least has the following advantages:
(1) The detailed methylated CpG sites of promoter regions of BMP3 and NDRG4 genes in the Chinese population are provided and can be as a biomarker for detecting CRC and/or AA.
(2) The kit is more suitable for CRC and AA detection in Chinese population than other similar products based on mt-sDNA, such as Cologuard®, because the present invention targets Chinese population specific methylation CpG sites, while the previous products are directed to the white people population.
(3) The sensitivity and specificity of CRC detection are higher compared to other similar products based on mt-sDNA.
(4) The sensitivity and specificity of AA detection are significantly improved compared to other similar products based on mt-sDNA.
(5) The method is noninvasive and easy for home sampling, therefore leads to good patient compliance, so that it can be widely used as a CRC and AA screening method. The method reduces the incidence and mortality due to CRC in the Asian population.

### EXAMPLES

### Example 1

Discovery that methylated CpG sites in the promoter regions of BMP3 and NDRG4 genes respectively in Chinese CRC and AA population.

### (1) Sample Collection

In total 191 colon FFPE tissue samples were collected from patients with CRC and AA confirmed by colonoscopy, including 50 colorectal cancer tissues and 49 paired adjacent normal tissues, 46 adenomas cancer tissues and 46 paired adjunct normal tissues.

### (2) DNA Extraction

Genomic DNAs were extracted from FFPE samples with the TaKaRa MiniBEST FFPE DNA Extraction Kit (catalogue number: 9782). The detailed operation steps are described as follows:
i. Scrap 30 mg paraffin section tissue with a sterile scalpel and remove excess paraffin.
ii. Put the paraffin section tissue in a 1.5 mL centrifuge tube, and add 500 μL of buffer DP, Mix and incubate in water at 80° C. for 1 minute, and then vortex for 10 seconds. Add 180 μL of Buffer GL and vortex.
iii. The mixture was centrifuged at 12,000 rpm for 1 minute at room temperature, and then the solution was formed into two layers (upper oil phase, lower aqueous phase). Add 20 μL of Proteinase K (20 mg/mL) and 10 μL of RNase(10 mg/mL) to the lower aqueous phase, and mix thoroughly with pipette up and down gently. Be careful not to disturb the layers. And then water bath at 56° C. for 1 hours.
iv. The solution of the previous step was incubated at 90° C. for 30 minutes and cooled to room temperature. And then add 200 μL of Buffer GB and 200 μL of 100% ethanol to the solution, vortex for 10 seconds. Centrifuge at 12,000 rpm for 1 minute at room template, and the solution was formed into two layers (upper oil phase, lower aqueous phase)
v. Put a spin column in a collection tube, and add lower aqueous phase of the previous step into the spin column. Be careful not to disturb the layers, and centrifuge at 12,000 rpm for 2 minutes at room temperature, and then discard the waste.
vi. Add 500 μL of Buffer WA to the spin column, and centrifuge at 12,000 rpm for 1 minute at room temperature, and then discard the waste.
vii. Add 500 μL of Buffer WB to the spin column, centrifuge at 12,000 rpm for 1 minute at room temperature, and then discard the waste. And repeat once.
viii. Put the spin column in the collection tube, centrifuge at 12,000 rpm for 2 minutes at room temperature.
ix. Put the spin column in a new 1.5 mL centrifuge tube, add 50-100 μL of sterilized water or elution buffer to the center of the spin column membrane, and then place it at room temperature for 5 minutes.
x. Centrifuge at 12,000 rpm for 2 minutes at room temperature, and DNA is eluted.
xi. The eluted DNA is quantified with Nanodrop 2000 fluorometer, and stored at −20° C. for use.

### (3) The Prediction of CpG Islands in Promoter Regions of BMP3 and NDRG4 Genes and Primer Designing for Amplicon Sequencing.

i. The Prediction of CpG Islands of Promoter Regions of BMP3 and NDRG4 Genes.

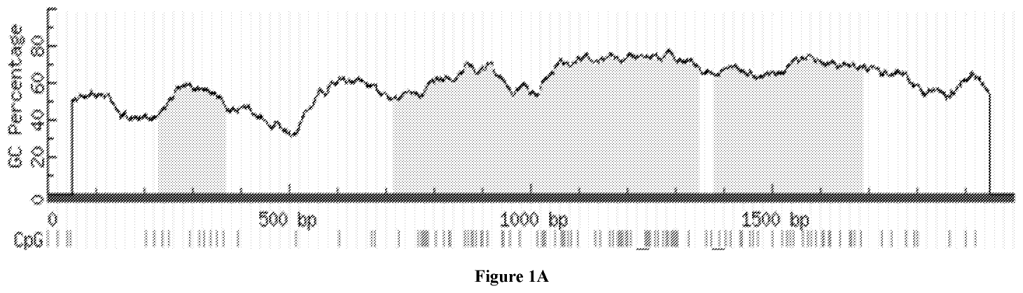

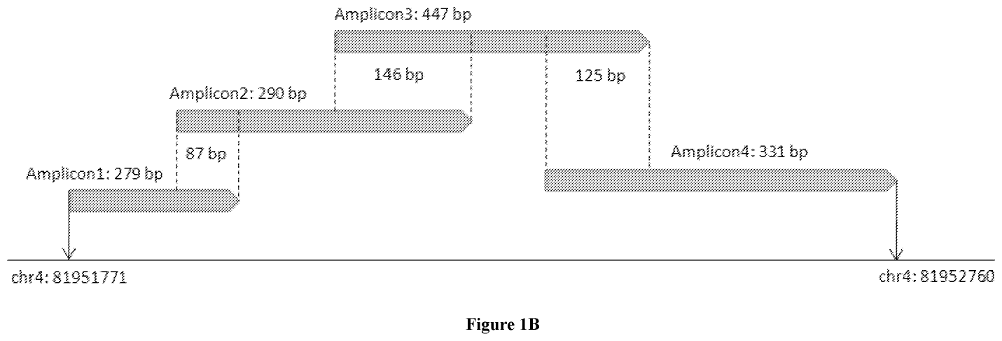

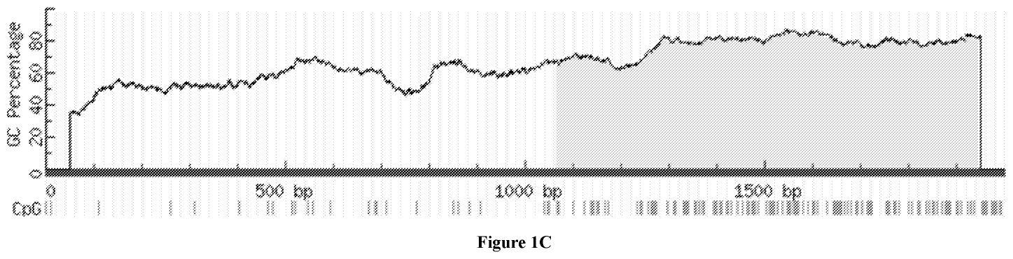

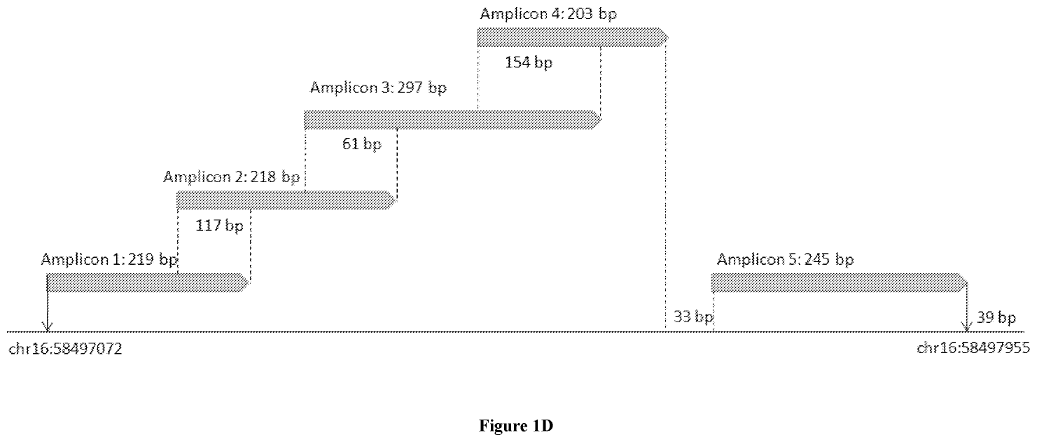

The promoter sequences of BMP3 and NDRG4 genes were downloaded, including DNA sequences approximately 1000-1500 bp upstream from transcription start site (TSS) and 5′UTR region. The CpG islands of the sequences were predicted with MethPrimer software (www.urogene.org/methprimer/). As shown in Figure A to FIG. 1D, two larger ones of the three CpG islands of BMP3 gene are located at approximately 400 bp upstream from TSS and whole 5′UTR region(chr4: 81951752-81952760), and only one CpG island of NDRG4 gene is located at approximately 500 bp upstream from TSS and partial 5′UTR region(chr16:58497061-58497938). The build of human reference genome is GRCh37/hg19.

ii. Primer Designing to Amplify Sequence of CpG Islands Predicted of BMP3 and NDRG4 Genes.

Based on the length of the sequences to be amplified and read length of sequencing, four and five pairs of primers were designed for BMP3 and NDRG4 genes, respectively. There is as much overlap as possible between adjacent amplicons so that the CpG islands of the two genes can be sequenced thoroughly. The primers of the two genes are listed in Table 1, and the relative position of amplicons of two genes is shown in FIG. 1A to FIG. 1D.

TABLE 1
the primers for amplicon sequencing of BMP3 and NDRG4 genes

| Gene | Amplicon | Primer | Sequence ID | Sequence (5′ to 3′) |
|---|---|---|---|---|
| BMP3 | Amplicon 1 | Forward Primer 1 | SEQ ID NO.: 47 | AGTTTGGTGTAAGTTAAGAG |
| BMP3 | | Reverse Primer 1 | SEQ ID NO.: 48 | CTAACTCTATTTTAAACRCCA |
| BMP3 | Amplicon 2 | Forward Primer 2 | SEQ ID NO.: 49 | GTTTTAATTTTTGGAAAAGGTAA |
| BMP3 | | Reverse Primer 2 | SEQ ID NO.: 50 | ACCTAACAAATAAACTCTTCC |
| BMP3 | Amplicon 3 | Forward Primer 3 | SEQ ID NO.: 51 | GAAGGTATAGATAGATTTTGAA |
| BMP3 | | Reverse Primer 3 | SEQ ID NO.: 52 | CACCTAACACAACTTTACRAAACT |
| BMP3 | Amplicon 4 | Forward Primer 4 | SEQ ID NO.: 53 | GTATTTAGTTATGGTTGGGGYGAGTA |
| BMP3 | | Reverse Primer 4 | SEQ ID NO.: 54 | CTCACCTACTACTACCGCCCR |
| NDRG4 | Amplicon 1 | Forward Primer 1 | SEQ ID NO.: 55 | AGGTTTTTGAGTTTTTTGGTTTTTTT |
| NDRG4 | | Reverse Primer 1 | SEQ ID NO.: 56 | CCCTCCAAACCCCCTATAAC |
| NDRG4 | Amplicon 2 | Forward Primer 2 | SEQ ID NO.: 57 | GGATGGGGATGTTTTTGTAG |
| NDRG4 | | Reverse Primer 2 | SEQ ID NO.: 58 | RGRGAAACCTAAAAAACACC |
| NDRG4 | Amplicon 3 | Forward Primer 3 | SEQ ID NO.: 59 | GYGGAGYGGGTGAGAAGT |
| NDRG4 | | Reverse Primer 3 | SEQ ID NO.: 60 | CRAACAACCAAAAACCCCTC |
| NDRG4 | Amplicon 4 | Forward Primer 4 | SEQ ID NO.: 61 | GTTYGTTYGGGATTAGTTTTAGG |
| NDRG4 | | Reverse Primer 4 | SEQ ID NO.: 62 | CRCAAACRAAAAACRAAAC |
| NDRG4 | Amplicon 5 | Forward Primer 5 | SEQ ID NO.: 63 | GYGGYGTTTTYGTTTTTG |
| NDRG4 | | Reverse Primer 5 | SEQ ID NO.: 64 | CRACRACTAAAAATCCCCAA |

DNA samples were subjected to bisulfite treatment as described below.

### (4) Bisulfite Treatment

i. The extracted DNA is put at room temperature to thaw, and the DNA concentration is diluted to 20 ng/μL. Add 40 μL of the diluted DNA to a 1.5 mL centrifuge tube, and then add 4 μL of 3M NaOH solution, incubate at 42° C. for 20 minutes.
ii. Add 400 μL of a conversion solution, mix, and incubate at 50° C. of 16 hours in the dark.
iii. Add 550 μL of binding solution, mix, and transfer the solution to a DNA purification column. Centrifuge at 13,000 rpm for 90 seconds and discard the waste. Centrifuge again for 3 minutes and discard the waste.
iv. Add 600 μL of 90′ ethanol to the DNA purification column, centrifuge at 13,000 rpm for 90 seconds, and discard the waste. Centrifuge again at 13,000 rpm for 15 seconds.
v. Add 300 μL of desulfurization solution (0.3 M NaOH of 90% ethanol solution) to the DNA purification, put it at room temperature for 30 minutes. Centrifuge at 13,000 rpm for 90 seconds and discard the waste.
vi. Add 600 μL of 90% ethanol, centrifuge at 13,000 rpm for 90 seconds, and discard the waste. Repeat this step once, and centrifuge again at 13,000 rpm for 3 minutes.
vii. Put the DNA purification column into a new 1.5 mL centrifuge tube, and add 40 μL of eluate. Incubate the tube at 50° C. for 30 minutes, and centrifuge at 13,000 rpm for 90 seconds. Store the converted DNA solution at −20° C. for use.

### (5) Library Preparation and Amplicon Sequencing

(a) Multiplex PCR Amplification
i. Prepare PCR master mixture as follows:

TABLE 2

| No. | reagents | Final concentration |
|---|---|---|
| 1 | 2 × QIAGNE Multiplex PCR Master | 1× |
| | Mix(QIAGEN, Catalogue NO.: 206143) | |
| 2 | 10 × primer mix(2 μM each) | 0.2 μM |
| 3 | RNase-free Water | Up to 40 μL |

ii. Vortex the mixture gently and pipette 40 μL of which to each PCR tubes, and then add 10 μL of bisulfate treated DNA.
iii. Vortex gently and PCR amplification was preformed as follows: one cycle of denatured at 95° C. for 15 minutes, 35 cycles of denatured at 94° C. for 30 seconds, annealed at 55° C. for 90 seconds, and elongated at 72° C. for 90 seconds, one cycle of elongated at 72° C. for 10 minutes, and finally held at 4° C. forever.
iv. 5 μL of PCR products were blended with 6× loading buffer (TakaRa, catalogue No.:9156) and loaded to 1% (w/v) agarose gel with a control of DL2000 DNA marker (TakaRa, catalogue No.:3427Q), and electrophoresis at 120V for 40 minutes.
v. If there was a non-specific amplification, it was necessary to recovered remaining 45 μL of PCR products after electrophoresis according to the kit instructions (QIAGEN, catalogue No.: 28704).

(b) Purification and Quantification of PCR Products

PCR product was purified with MinElute PCR Purification Kit according to the kit instructions (QIAGEN, Catalogue No.: 28004). Purified PCR product was quantified with Qubit™ dsDNA BR Assay Kit (Catalogue No.: Q32850).

(c) Adapter Ligation

Adapters were ligated with purified PCR products with NEBNext Quick Ligation Module (NEB, Catalogue Number: E6056L), and the reaction mixture were prepared as follows:

TABLE 3
Reaction Mixture for adapter litigation

| | | Volume |
|---|---|---|
| No. | Reagents | (μL)/reaction |
| 1 | NEBNext Quick Ligation Reaction Buffer(5×) | 10 |
| 2 | Adapter | 5 |
| 3 | Quick T4 DNA Ligase | 5 |
| 4 | RNase-free Water | 10 |

Add 30 μL of mixture to each PCR tubes and then add 20 μL of purified PCR products respectively. The solution were mixed gently, incubated at 20° C. for 15 minutes and held at 4° C. for 10 minutes.

(d) Purification of Ligation Products
i. Put Agencourt AMpure XP beads (Beckman, Catalogue number: A63882) to room temperature for use.
ii. Centrifuge the PCR tubes at 280 g for 1 minute at 20° C., and transfer the 50 μL of ligation products to a new 96-well PCR plate.
iii. Vortex the AMpure XP beads for 30 seconds to be dispersed evenly, and add 56 μL of beads to each well of the PCR plate. Pipette the mixture gently for 10 times to mix, and keep still at room temperature for 5 minutes.
iv. Place the 96-well PCR plate on a 96-well magnetic plate, keep it sill for 2 minutes until the supernatant was clear. Keep the 96 well PCR plate on the 96-well magnetic plate and remove the supernatant, and then add 200 μL of freshly prepared 80% ethanol to each wells, and incubate at room temperature for 30 seconds, and remove the supernatant.
v. Keep the 96-well PCR plate on the 96-well magnetic plate and add 200 μL of freshly prepared 80% ethanol to each wells. And then incubate at room temperature for 30 seconds. Discard the supernatant and remove the residual ethanol with 10 μL pipette.
vi. Keep the 96-well PCR plate on the magnetic plate and let the beads dry naturally for 10 minutes.
vii. Remove the 96-well PCR plate from the magnetic plate and add 27.5 μL of 10 mM Tris (pH 8.5) to each wells of the 96-well plate. Pipette up and down gently to mix beads and Tris for 10 times until the beads were dispersed thoroughly. Keep the plate still at room temperature for 2 minutes.
viii. Place the 96-well PCR plate on the magnetic plate and still for 2 minutes until the supernatant is clear. Pipette 25 μL of the supernatant to a new PCR tube and store it at −20° C. for use.

(e) PCR Amplification and Product Purification

PCR master mixture was prepared as follows and mixed gently. Add 40 μL of mixture to each PCR tubes and then add 5 μL of purified ligation products.

TABLE 4
PCR reaction mixture

| NO. | Reagents | Final concentration |
|---|---|---|
| 1 | NEBNext Ultra Q5 Master Mix | 1× |
| | (NEB, catalogue number: M0544) | |
| 2 | Forward Primer(10 μM) | 1 μM |
| 3 | Reverse Primer(10 μM) | 1 μM |
| 4 | Nuclease-free water | Up to 40 10 μL |

Vortex the PCR tubes gently and centrifuge briefly, and carries PCR reaction as the following condition: one cycle of denaturing at 98° C. for 30 seconds, 8 to 15 cycles of denaturing at 98° C. for 10 seconds and elongating at 65° C. for 75 seconds, one cycle of denaturing at 65° C. for 5 minutes, and finally hold at 4° C. PCR products were purified according to (d) Purification of ligation products.

(f) Detection and Sequencing of Library

The concentration and size distribution of purified PCR products were analyzed with Qubit™ dsDNA BR Assay kit (catalogue number: Q32850) and the Agilent 2100 Bioanalyzer Instrument, respectively. Libraries were pooling with equal molar concentration and sequenced on Illumina HiSeq2500 with read length of PE125.

(g) Sequencing Data Analysis

Raw data was demultiplexed according sample indexes and sequencing reads were mapped to reference gene sequence of BMP3 and NDRG4 with SHRiMP V2.04 software. Methylated CpG sites were identified based on mapping results. And finally, It founded that 26 and 39 CpG sites of BMP3 and NDRG4 genes were hypermethylated at CRC and AA tissues compared to adjacent normal tissues (p<0.05), which indicated that these methylated CpG sites can be a DNA biomarker for early diagnosis of CRC and AA.

TABLE 5
Methylated frequency of CpG sites of BMP3 gene in CRC tissues and adjacent normal tissues.

| | | | The number of | | The number of | | |
|---|---|---|---|---|---|---|---|
| | | | methylated | | methylated | | |
| | | Location to | samples divided | | samples divided | | |
| | Chromosome | transcript | by the total | Methylation | by the total | Methylation | |
| CpG | coordinate | start site | number of samples- | percentage- | number of samples- | percentage- | P- |
| sites | (hg19) | (NM_001201.3) | CRC | CRC | Normal | Normal | value |
| #1 | 81952078 | −41 | 34/50 | 0.68 | 2/49 | 0.04 | <0.01 |
| #2 | 81952099 | −20 | 38/50 | 0.76 | 2/49 | 0.04 | <0.01 |
| #3 | 81952135 | 17 | 33/50 | 0.66 | 1/49 | 0.02 | <0.01 |
| #4 | 81952146 | 28 | 34/50 | 0.68 | 2/49 | 0.04 | <0.01 |
| #5 | 81952149 | 31 | 34/50 | 0.68 | 1/49 | 0.02 | <0.01 |
| #6 | 81952151 | 33 | 35/50 | 0.7 | 2/49 | 0.04 | <0.01 |
| #7 | 81952172 | 54 | 33/50 | 0.66 | 0/49 | 0.00 | <0.01 |
| #8 | 81952186 | 68 | 33/50 | 0.66 | 1/49 | 0.02 | <0.01 |
| #9 | 81952189 | 71 | 36/50 | 0.72 | 1/49 | 0.02 | <0.01 |
| #10 | 81952193 | 75 | 37/50 | 0.74 | 0/49 | 0.00 | <0.01 |
| #11 | 81952198 | 80 | 34/50 | 0.68 | 1/49 | 0.02 | <0.01 |
| #12 | 81952205 | 87 | 37/50 | 0.74 | 2/49 | 0.04 | <0.01 |
| #13 | 81952207 | 89 | 37/50 | 0.74 | 2/49 | 0.04 | <0.01 |
| #14 | 81952218 | 100 | 34/50 | 0.68 | 1/49 | 0.02 | <0.01 |
| #15 | 81952220 | 102 | 37/50 | 0.74 | 2/49 | 0.04 | <0.01 |
| #16 | 81952255 | 137 | 35/50 | 0.7 | 0/49 | 0.00 | <0.01 |
| #17 | 81952266 | 148 | 33/50 | 0.66 | 1/49 | 0.02 | <0.01 |
| #18 | 81952285 | 167 | 38/50 | 0.76 | 1/49 | 0.02 | <0.01 |
| #19 | 81952293 | 175 | 36/50 | 0.72 | 1/49 | 0.02 | <0.01 |
| #20 | 81952302 | 184 | 35/50 | 0.7 | 1/49 | 0.02 | <0.01 |
| #21 | 81952306 | 188 | 33/50 | 0.66 | 1/49 | 0.02 | <0.01 |
| #22 | 81952308 | 190 | 36/50 | 0.72 | 0/49 | 0.00 | <0.01 |
| #23 | 81952313 | 195 | 35/50 | 0.7 | 0/49 | 0.00 | <0.01 |
| #24 | 81952320 | 202 | 35/50 | 0.7 | 2/49 | 0.04 | <0.01 |
| #25 | 81952324 | 206 | 38/50 | 0.76 | 2/49 | 0.04 | <0.01 |
| #26 | 81952330 | 212 | 33/50 | 0.66 | 2/49 | 0.04 | <0.01 |

TABLE 6
Methylated frequency of CpG sites of BMP3 gene in AA tissues and adjacent normal tissues.

| | | | The number | | The number | | |
|---|---|---|---|---|---|---|---|
| | | | of methylated | | of methylated | | |
| | | Location to | samples divided | | samples divided | | |
| | Chromosome | transcript | by the total | Methylation | by the total | Methylation | |
| CpG | coordinate | start site | number of | percentage- | number of | percentage- | P |
| sites | (hg19) | (NM_001201.3) | samples-AA | AA | samples-AA | Normal | value |
| #1 | 81952078 | −41 | 30/50 | 0.60 | 2/49 | 0.04 | <0.01 |
| #2 | 81952099 | −20 | 29/50 | 0.58 | 1/49 | 0.02 | <0.01 |
| #3 | 81952135 | 17 | 32/50 | 0.64 | 2/49 | 0.04 | <0.01 |
| #4 | 81952146 | 28 | 29/50 | 0.58 | 2/49 | 0.04 | <0.01 |
| #5 | 81952149 | 31 | 30/50 | 0.60 | 2/49 | 0.04 | <0.01 |
| #6 | 81952151 | 33 | 32/50 | 0.64 | 0/49 | 0.00 | <0.01 |
| #7 | 81952172 | 54 | 32/50 | 0.64 | 2/49 | 0.04 | <0.01 |
| #8 | 81952186 | 68 | 32/50 | 0.64 | 2/49 | 0.04 | <0.01 |
| #9 | 81952189 | 71 | 28/50 | 0.56 | 0/49 | 0.00 | <0.01 |
| #10 | 81952193 | 75 | 29/50 | 0.58 | 1/49 | 0.02 | <0.01 |
| #11 | 81952198 | 80 | 29/50 | 0.58 | 0/49 | 0.00 | <0.01 |
| #12 | 81952205 | 87 | 31/50 | 0.62 | 1/49 | 0.02 | <0.01 |
| #13 | 81952207 | 89 | 29/50 | 0.58 | 0/49 | 0.00 | <0.01 |
| #14 | 81952218 | 100 | 32/50 | 0.64 | 1/49 | 0.02 | <0.01 |
| #15 | 81952220 | 102 | 28/50 | 0.56 | 1/49 | 0.02 | <0.01 |
| #16 | 81952255 | 137 | 29/50 | 0.58 | 0/49 | 0.00 | <0.01 |
| #17 | 81952266 | 148 | 31/50 | 0.62 | 0/49 | 0.00 | <0.01 |
| #18 | 81952285 | 167 | 32/50 | 0.64 | 1/49 | 0.02 | <0.01 |
| #19 | 81952293 | 175 | 33/50 | 0.66 | 0/49 | 0.00 | <0.01 |
| #20 | 81952302 | 184 | 32/50 | 0.64 | 1/49 | 0.02 | <0.01 |
| #21 | 81952306 | 188 | 30/50 | 0.60 | 0/49 | 0.00 | <0.01 |
| #22 | 81952308 | 190 | 31/50 | 0.62 | 1/49 | 0.02 | <0.01 |
| #23 | 81952313 | 195 | 29/50 | 0.58 | 1/49 | 0.02 | <0.01 |
| #24 | 81952320 | 202 | 32/50 | 0.64 | 1/49 | 0.02 | <0.01 |
| #25 | 81952324 | 206 | 31/50 | 0.62 | 2/49 | 0.04 | <0.01 |
| #26 | 81952330 | 212 | 32/50 | 0.64 | 1/49 | 0.02 | <0.01 |

TABLE 7
Methylated frequency of CpG sites of NDRG4 gene in CRC tissues and adjacent normal tissues.

| | | | | | The number | | |
|---|---|---|---|---|---|---|---|
| | | | The number | | of methylated | | |
| | | | of methylated | | samples divided | | |
| | | Location to | samples divided | | by the total | | |
| | Chromosome | transcript | by the total | Methylation | number of | Methylation | |
| CpG | coordinate | start site | number of | percentage- | samples- | percentage- | P |
| sites | (hg19) | (NM_020465.3) | samples-CRC | CRC | normal tissue | Normal | value |
| #1 | chr16: 58497349 | −200 | 38/50 | 0.75 | 1/49 | 0.02 | <0.01 |
| #2 | chr16: 58497352 | −197 | 35/50 | 0.70 | 2/49 | 0.04 | <0.01 |
| #3 | chr16: 58497358 | −191 | 35/50 | 0.70 | 2/49 | 0.04 | <0.01 |
| #4 | chr16: 58497360 | −189 | 39/50 | 0.78 | 1/49 | 0.03 | <0.01 |
| #5 | chr16: 58497365 | −184 | 39/50 | 0.78 | 1/49 | 0.03 | <0.01 |
| #6 | chr16: 58497369 | −180 | 39/50 | 0.77 | 1/49 | 0.02 | <0.01 |
| #7 | chr16: 58497388 | −161 | 37/50 | 0.74 | 1/49 | 0.02 | <0.01 |
| #8 | chr16: 58497390 | −159 | 37/50 | 0.73 | 1/49 | 0.03 | <0.01 |
| #9 | chr16: 58497393 | −156 | 40/50 | 0.79 | 1/49 | 0.03 | <0.01 |
| #10 | chr16: 58497395 | −154 | 37/50 | 0.74 | 1/49 | 0.02 | <0.01 |
| #11 | chr16: 58497402 | −147 | 38/50 | 0.76 | 1/49 | 0.03 | <0.01 |
| #12 | chr16: 58497406 | −143 | 39/50 | 0.78 | 1/49 | 0.02 | <0.01 |
| #13 | chr16: 58497414 | −135 | 37/50 | 0.74 | 1/49 | 0.03 | <0.01 |
| #14 | chr16: 58497418 | −131 | 36/50 | 0.72 | 0/49 | 0.00 | <0.01 |
| #15 | chr16: 58497425 | −124 | 36/50 | 0.72 | 1/49 | 0.02 | <0.01 |
| #16 | chr16: 58497438 | −111 | 36/50 | 0.72 | 2/49 | 0.04 | <0.01 |
| #17 | chr16: 58497440 | −109 | 36/50 | 0.71 | 1/49 | 0.02 | <0.01 |
| #18 | chr16: 58497449 | −100 | 37/50 | 0.73 | 2/49 | 0.04 | <0.01 |
| #19 | chr16: 58497458 | −91 | 39/50 | 0.77 | 2/49 | 0.04 | <0.01 |
| #20 | chr16: 58497460 | −89 | 37/50 | 0.74 | 1/49 | 0.03 | <0.01 |
| #21 | chr16: 58497462 | −87 | 38/50 | 0.75 | 1/49 | 0.03 | <0.01 |
| #22 | chr16: 58497470 | −79 | 35/50 | 0.70 | 1/49 | 0.02 | <0.01 |
| #23 | chr16: 58497472 | −77 | 36/50 | 0.71 | 2/49 | 0.04 | <0.01 |
| #24 | chr16: 58497477 | −72 | 36/50 | 0.72 | 1/49 | 0.03 | <0.01 |
| #25 | chr16: 58497481 | −68 | 37/50 | 0.74 | 1/49 | 0.02 | <0.01 |
| #26 | chr16: 58497499 | −50 | 37/50 | 0.73 | 1/49 | 0.02 | <0.01 |
| #27 | chr16: 58497505 | −44 | 40/50 | 0.79 | 2/49 | 0.04 | <0.01 |
| #28 | chr16: 58497510 | −39 | 40/50 | 0.79 | 1/49 | 0.02 | <0.01 |
| #29 | chr16: 58497512 | −37 | 40/50 | 0.79 | 2/49 | 0.04 | <0.01 |
| #30 | chr16: 58497517 | −32 | 35/50 | 0.70 | 1/49 | 0.02 | <0.01 |
| #31 | chr16: 58497521 | −28 | 39/50 | 0.77 | 1/49 | 0.02 | <0.01 |
| #32 | chr16: 58497531 | −18 | 38/50 | 0.76 | 1/49 | 0.03 | <0.01 |
| #33 | chr16: 58497542 | −7 | 36/50 | 0.71 | 1/49 | 0.02 | <0.01 |
| #34 | chr16: 58497544 | −5 | 37/50 | 0.74 | 1/49 | 0.03 | <0.01 |
| #35 | chr16: 58497550 | 2 | 37/50 | 0.74 | 0/49 | 0.00 | <0.01 |
| #36 | chr16: 58497556 | 8 | 38/50 | 0.75 | 1/49 | 0.03 | <0.01 |
| #37 | chr16: 58497558 | 10 | 39/50 | 0.78 | 2/49 | 0.04 | <0.01 |
| #38 | chr16: 58497561 | 13 | 36/50 | 0.71 | 2/49 | 0.04 | <0.01 |
| #39 | chr16: 58497566 | 18 | 35/50 | 0.70 | 1/49 | 0.03 | <0.01 |

TABLE 8
Methylated frequency of CpG sites of NDRG4 gene in AA tissues and adjacent normal tissues.

| | | | | | The number | | |
|---|---|---|---|---|---|---|---|
| | | | The number | | of methylated | | |
| | | | of methylated | | samples divided | | |
| | | Location to | samples divided | | by the total | | |
| | Chromosome | transcript | by the total | Methylation | number of | Methylation | |
| CpG | coordinate | start site | number of | percentage- | samples- | percentage- | P |
| sites | (hg19) | (NM_020465.3) | samples-AA | AA | normal tissue | Normal | value |
| #1 | chr16: 58497349 | −200 | 28/46 | 0.60 | 1/46 | 0.02 | <0.01 |
| #2 | chr16: 58497352 | −197 | 28/46 | 0.60 | 1/46 | 0.02 | <0.01 |
| #3 | chr16: 58497358 | −191 | 27/46 | 0.59 | 1/46 | 0.03 | <0.01 |
| #4 | chr16: 58497360 | −189 | 29/46 | 0.62 | 1/46 | 0.03 | <0.01 |
| #5 | chr16: 58497365 | −184 | 30/46 | 0.65 | 2/46 | 0.04 | <0.01 |
| #6 | chr16: 58497369 | −180 | 28/46 | 0.61 | 2/46 | 0.04 | <0.01 |
| #7 | chr16: 58497388 | −161 | 29/46 | 0.64 | 1/46 | 0.03 | <0.01 |
| #8 | chr16: 58497390 | −159 | 29/46 | 0.64 | 1/46 | 0.03 | <0.01 |
| #9 | chr16: 58497393 | −156 | 30/46 | 0.65 | 1/46 | 0.02 | <0.01 |
| #10 | chr16: 58497395 | −154 | 28/46 | 0.60 | 2/46 | 0.04 | <0.01 |
| #11 | chr16: 58497402 | −147 | 29/46 | 0.62 | 1/46 | 0.03 | <0.01 |
| #12 | chr16: 58497406 | −143 | 29/46 | 0.62 | 1/46 | 0.03 | <0.01 |
| #13 | chr16: 58497414 | −135 | 28/46 | 0.61 | 1/46 | 0.02 | <0.01 |
| #14 | chr16: 58497418 | −131 | 30/46 | 0.65 | 1/46 | 0.02 | <0.01 |
| #15 | chr16: 58497425 | −124 | 29/46 | 0.64 | 2/46 | 0.04 | <0.01 |
| #16 | chr16: 58497438 | −111 | 29/46 | 0.64 | 1/46 | 0.02 | <0.01 |
| #17 | chr16: 58497440 | −109 | 29/46 | 0.62 | 2/46 | 0.04 | <0.01 |
| #18 | chr16: 58497449 | −100 | 29/46 | 0.64 | 1/46 | 0.03 | <0.01 |
| #19 | chr16: 58497458 | −91 | 29/46 | 0.62 | 1/46 | 0.02 | <0.01 |
| #20 | chr16: 58497460 | −89 | 27/46 | 0.59 | 1/46 | 0.03 | <0.01 |
| #21 | chr16: 58497462 | −87 | 29/46 | 0.62 | 1/46 | 0.02 | <0.01 |
| #22 | chr16: 58497470 | −79 | 29/46 | 0.64 | 1/46 | 0.02 | <0.01 |
| #23 | chr16: 58497472 | −77 | 30/46 | 0.65 | 2/46 | 0.04 | <0.01 |
| #24 | chr16: 58497477 | −72 | 30/46 | 0.65 | 2/46 | 0.04 | <0.01 |
| #25 | chr16: 58497481 | −68 | 28/46 | 0.60 | 2/46 | 0.04 | <0.01 |
| #26 | chr16: 58497499 | −50 | 29/46 | 0.63 | 1/46 | 0.02 | <0.01 |
| #27 | chr16: 58497505 | −44 | 28/46 | 0.61 | 1/46 | 0.02 | <0.01 |
| #28 | chr16: 58497510 | −39 | 27/46 | 0.59 | 1/46 | 0.02 | <0.01 |
| #29 | chr16: 58497512 | −37 | 30/46 | 0.65 | 2/46 | 0.04 | <0.01 |
| #30 | chr16: 58497517 | −32 | 29/46 | 0.62 | 2/46 | 0.04 | <0.01 |
| #31 | chr16: 58497521 | −28 | 29/46 | 0.64 | 1/46 | 0.02 | <0.01 |
| #32 | chr16: 58497531 | −18 | 29/46 | 0.62 | 2/46 | 0.04 | <0.01 |
| #33 | chr16: 58497542 | −7 | 29/46 | 0.64 | 1/46 | 0.02 | <0.01 |
| #34 | chr16: 58497544 | −5 | 29/46 | 0.63 | 1/46 | 0.02 | <0.01 |
| #35 | chr16: 58497550 | 2 | 29/46 | 0.62 | 1/46 | 0.02 | <0.01 |
| #36 | chr16: 58497556 | 8 | 28/46 | 0.61 | 1/46 | 0.03 | <0.01 |
| #37 | chr16: 58497558 | 10 | 30/46 | 0.65 | 1/46 | 0.03 | <0.01 |
| #38 | chr16: 58497561 | 13 | 29/46 | 0.63 | 1/46 | 0.02 | <0.01 |
| #39 | chr16: 58497566 | 18 | 30/46 | 0.65 | 1/46 | 0.02 | <0.01 |

### Example 2

### Comparison of Differential Methylation CpG Sites or BMP3 and NDRG4 Genes to Related to CRC Among Different Races.

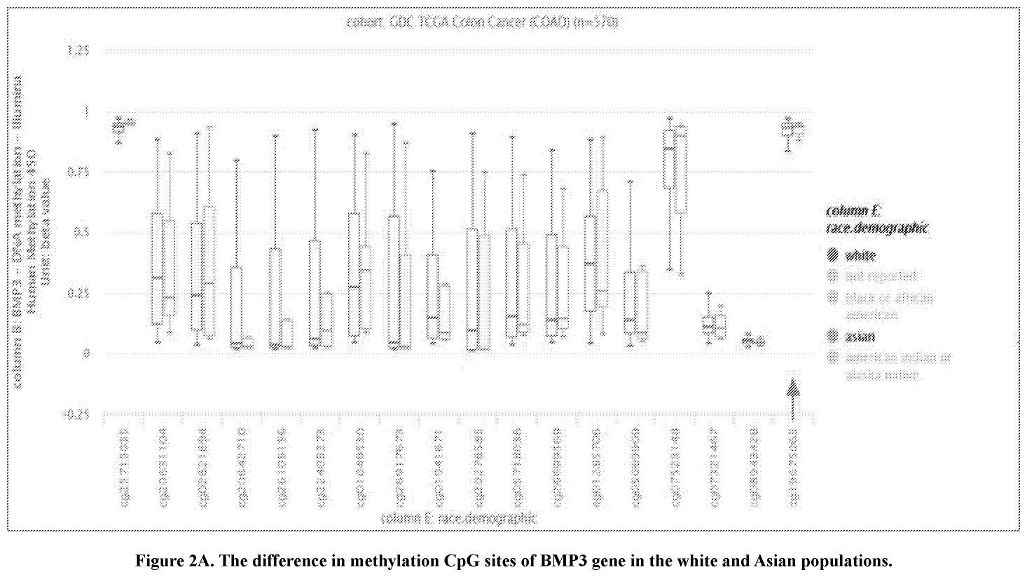

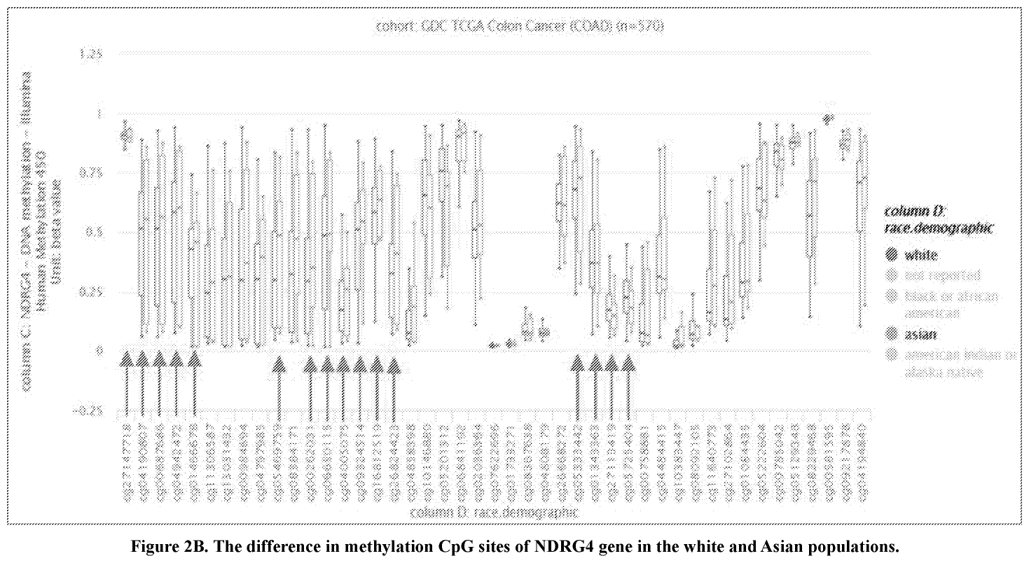

We analyzed the methylation microarray data of BMP3 and NDRG4 genes in TCGA database (Illumina Human Methylation 450 data) and found that there are five significantly different methylation CpG sites in the promoter region of NDRG4 genes between the white and Asian populations (FIG. 2 and Table 9). In order to further verify the differential methylation CpG sites, tissue and blood samples were collected from 106 CRC and AA patients in China. The DNAs were extracted and treated with bisulfate. The promoter regions of BMP3 and NDRG4 genes were amplified and sequenced. We analyzed the sequencing data and found indeed that hypermethylation CpG sites of Asian population are different from that of the white in TCGA database, and the hypermethylation CpG sites are different between CRC and AA tissue samples. According to the different methylation CpG sites, we developed a detection kit specifically for CRC and AA screening in the Asian population (e.g. Chinese population).

TABLE 9
the difference in methylation CpG sites of BMP3 and NDRG4 gene in the white and Asian populations.

| | | | | | | Methylation |
|---|---|---|---|---|---|---|
| | | Methylation | | Coordinate | Gene | percentage(White > |
| NO. | Gene | Site | One-way Anova | (hg19) | region | Asian) |
| 1 | BMP3 | cg19675063 | p = 0.001735, (f = 4.408) | 81975265 | Exon3 | Y |
| 2 | NDRG4 | cg27147718 | p = 0.00004290, (f = 6.558) | 58496542 | promoter | Y |
| 3 | NDRG4 | cg04190807 | p = 0.01761, (f = 3.036) | 58497230 | promoter | N |
| 4 | NDRG4 | cg00687686 | p = 0.01657, (f = 3.072) | 58497236 | promoter | N |
| 5 | NDRG4 | cg04942472 | p = 0.01955, (f = 2.973) | 58497239 | promoter | N |
| 6 | NDRG4 | cg01466678 | p = 0.02327, (f = 2.867) | 58497395 | promoter | N |
| 7 | NDRG4 | cg05469759 | p = 0.03612, (f = 2.599) | 58498456 | Intron1 | N |
| 8 | NDRG4 | cg00262031 | p = 0.02585, (f = 2.803) | 58498574 | Intron1 | N |
| 9 | NDRG4 | cg06650115 | p = 0.01221, (f = 3.256) | 58498585 | Intron1 | N |
| 10 | NDRG4 | cg04005075 | p = 0.01938, (f = 2.978) | 58498636 | Intron1 | N |
| 11 | NDRG4 | cg09324514 | p = 0.0004509, (f = 5.194) | 58498710 | Intron1 | N |
| 12 | NDRG4 | cg16812519 | p = 0.01862, (f = 3.002) | 58498754 | Intron1 | N |
| 13 | NDRG4 | cg26824423 | p = 0.007544, (f = 3.543) | 58498818 | Intron1 | N |
| 14 | NDRG4 | cg05333442 | p = 0.003665, (f = 3.970) | 58533743 | Intron3 | N |
| 15 | NDRG4 | cg01343363 | p = 0.01099, (f = 3.319) | 58533808 | Intron3 | Y |
| 16 | NDRG4 | cg27113419 | p = 0.006302, (f = 3.649) | 58533979 | Intron3 | Y |
| 17 | NDRG4 | cg05725404 | p = 0.01145, (f = 3.294) | 58534157 | Intron3 | N |

### Example 3

### Screening of Primers and Probes for BMP3 and NDRG4 Genes

### (1) Designing and Selection of Primers and Probes of BMP3 and NDRG4 Genes.

qPCR Primers and probes were designed based on the methylated CpG sites of BMP3 and NDRG4 genes. Three pairs of preferred primers and probes were identified. The preferred primers and probes are compared with several other candidate primes and probes of BMP3 and NDRG4 genes with positive controls and negative controls. The information of primers and probes is shown in Table 10.

TABLE 10
the information of preferred and remaining
primers and probes

| | | Primer/ | Primer/ | Sequence |
|---|---|---|---|---|
| Gene | Group | Probe | Probe ID | (5′ to 3′) |
| BMP3 | Preferred 1 | Forward | SEQ ID | TTTGAAAATATTCG |
| | | primer | NO.: 3 | GGTTATATACGTCG |
| | | | | C |
| BMP3 | Preferred 1 | Reverse | SEQ ID | ATAAACTCTTCCCC |
| | | primer | NO.: 4 | AACAACTACGCGAA |
| BMP3 | Preferred 1 | Probe | SEQ ID | AGCGTTGGAGTGGA |
| | | | NO.: 5 | GACGGCGTTCG |
| NDRG4 | Preferred 1 | Forward | SEQ ID | ATCGATCGGGGTGT |
| | | primer | NO.: 6 | TTTTTAGGTTTC |
| NDRG4 | Preferred 1 | Reverse | SEQ ID | CCTTCTACGCGACT |
| | | primer | NO.: 7 | AAAATACCCGAT |
| NDRG4 | Preferred 1 | Probe | SEQ ID | CGTCGCGGTTTTCG |
| | | | NO.: 8 | TTCGTTTTTTCGTT |
| | | | | CGT |
| BMP3 | Preferred 2 | Forward | SEQ ID | AATATTCGGGITAT |
| | | primer | NO.: 9 | ATACGTCGCGA |
| BMP3 | Preferred 2 | Reverse | SEQ ID | GCAACCTAACAAAT |
| | | primer | NO.: 10 | AAACTCTTCCCCAA |
| BMP3 | Preferred 2 | Probe | SEQ ID | TGGAGTGGAGACGG |
| | | | NO.: 11 | CGTTCGTAGCGT |
| KDRG4 | Preferred 2 | Forward | SEQ ID | GCGGGTGAGAAGTC |
| | | primer | NO.: 12 | GGC |
| NDRG4 | Preferred 2 | Reverse | SEQ ID | GTAACTTCCGCCTT |
| | | primer | NO.: 13 | CTACGC |
| NDRG4 | Preferred 2 | Probe | SEQ ID | TAGGTTTCGCGTCG |
| | | | NO.: 14 | CGGTTTTCGTT |
| BMP3 | Preferred 3 | Forward | SEQ ID | AATATTCGGGTTAT |
| | | primer | NO.: 15 | ATACGTCGCGATT |
| BMP3 | Preferred 3 | Reverse | SEQ ID | ACTTACTACGCTAA |
| | | printer | NO.: 16 | CCCAACG |
| BMP3 | Preferred 3 | Probe | SEQ ID | TAGCGTTGGAGTGG |
| | | | NO.: 17 | AGACGGCGTTCGTA |
| NDRG4 | Preferred 3 | Forward | SEQ ID | CGGTTTTCGTTCGT |
| | | primer | NO.: 18 | TTTTTCG |
| NDRG4 | Preferred 3 | Reverse | SEQ ID | AACCTAAAACTAAT |
| | | primer | NO.: 19 | CCCGAACGAACC |
| KDRG4 | Preferred 3 | Probe | SEQ ID | TCGTTTATCGGGTA |
| | | | NO.: 20 | TTTTAGTCGCGTAG |
| BMP3 | Control 1 | Forward | SEQ ID | GAGTGGAGACGGCG |
| | | primer | NO.: 21 | TTCGTA |
| BMP3 | Control 1 | Reverse | SEQ ID | CCACTTACTACGCT |
| | | primer | NO.: 22 | AACCCAACG |
| BMP3 | Control 1 | Probe | SEQ ID | CGGGTGAGGTTCGC |
| | | | NO.: 23 | GTAGTTGTTGGG |
| NDRG4 | Control 1, | Forward | SEQ ID | GGGTGTTTTTTAGG |
| | Control 2, | primer | NO.: 24 | TTTCGCGTC |
| | Control 3 | | | |
| NDRG4 | Control 1 | Reverse | SEQ ID | CGTAACTTCCGC |
| | | primer | NO.: 25 | CTTCTACGC |
| NDRG4 | Control 1, | Probe | SEQ ID | ACGCGACTAAAAT |
| | Control 2, | | NO.: 26 | ACCCGATAAACGA |
| | Control 3 | | | ACGAAAAAACGAAC |
| BMP3 | Control 2 | Forward | SEQ ID | TTAGGTTGCGTT |
| | | primer | NO.: 27 | GGGTTAGCG |
| BMP3 | Control 2 | Reverse | SEQ ID | ACTCCGAAAAC |
| | | primer | NO.: 28 | GCAAAAAACCG |
| BMP3 | Control 2 | Probe | SEQ ID | ATTCGGTCGCG |
| | | | NO.: 29 | TTTCGGGTTTC |
| | | | | GTGC |
| NDRG4 | Control 2 | Reverse | SEQ ID | GACCCGCGAAA |
| | | primer | NO.: 30 | CGATACCG |
| BMP3 | Control 3 | Forward | SEQ ID | TATTCGGGTTA |
| | | primer | NO.: 31 | TATACGTCGC |
| BMP3 | Control 3 | Reverse | SEQ ID | CTTACTACGCT |
| | | primer | NO.: 32 | AACCCAACG |
| BMP3 | Control 3 | Probe | SEQ ID | CCCAACAACTA |
| | | | NO.: 33 | CGCGAACCTCA |
| | | | | CCCG |
| NDRG4 | Control 3 | Reverse | SEQ ID | TCGCGCGTAAC |
| | | primer | NO.: 34 | TTCCGCCTT |

(2) Comparison Preferred Primers and Probes with Controls with Positive and Negative Controls of BMP3 and NDRG4 Genes.

Standard samples with different methylation ratios were formed by spiking positive control DNAs into negative control DNAs of BMP3 and NDRG4 genes respectively in different ratio (Table 11). Analytical sensitivity was compared by amplify the standard sample DNA with preferred and control primers and probes of BMP3 and NDRG4 genes respectively. Analytical specificity was compared by amplify negative control DNA of BMP3 and NDRG4 genes with preferred and control primers and probes of BMP3 and NDRG4 genes respectively, and the quantity of negative control DNAs is 104 copies, 105 copies, 10⁶ copies, 10′ copies and 10′ copies per reaction.

TABLE 11
standard sample of BMP3 and NDRG4 genes

| Methylation | Copy number of | Copy number of |
|---|---|---|
| ratio | methylated DNA | total DNA |
| 1 | 10⁵ | 10⁵ |
| 1/10 | 10⁴ | 10⁵ |
| 1/10² | 10³ | 10⁵ |
| 1/10³ | 10² | 10⁵ |
| 1/10⁴ | 10 | 10⁵ |

The master mixture was prepared as Table 5 and quantitative PCR reaction condition is denaturation at 95° C. for 1 minutes firstly, and then 50 cycles of denaturation at 95° C. for 20 seconds and elongation at 60° C. for 1 minutes.

TABLE 12
the final concentration of reagents of methylation qPCR

| No. | Reagents | Final concentration | |
|---|---|---|---|
| 1 | 10 × PCR buffer | 1× | |
| 2 | MgCl₂ | 2 | mM |
| 3 | dNTPs | 0.2 | mM |
| 4 | Taq DNA polymerase | 2 | U |
| 5 | Each primer | 0.75 | mM |
| 6 | Each probe | 0.25 | mM |
| 7 | DNA template | 2 | μL |

| | | |
|---|---|---|
| 8 | Ultrapure water | Up to 50 μL |

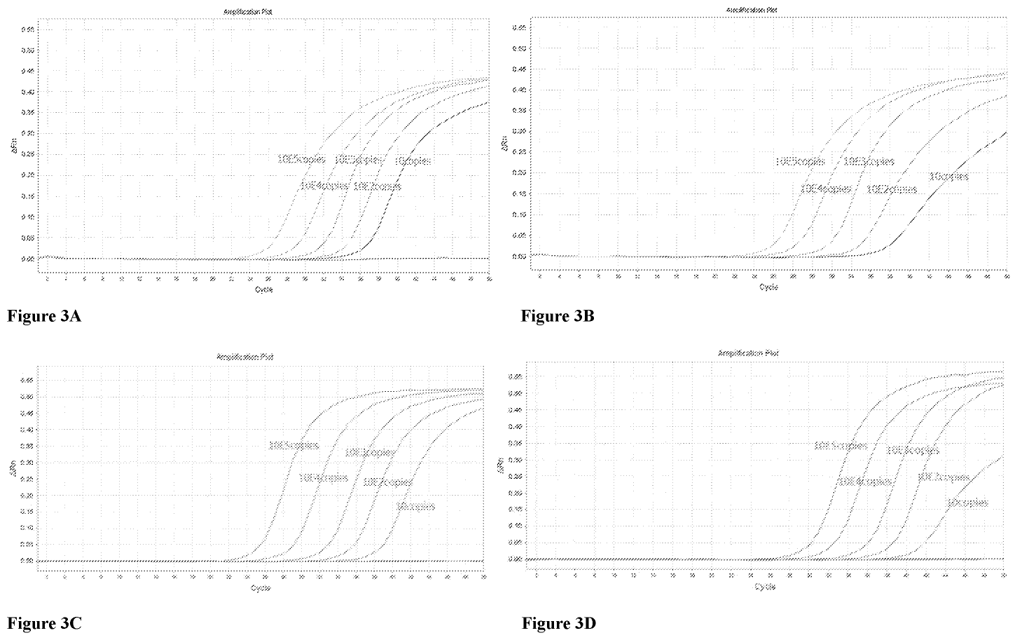

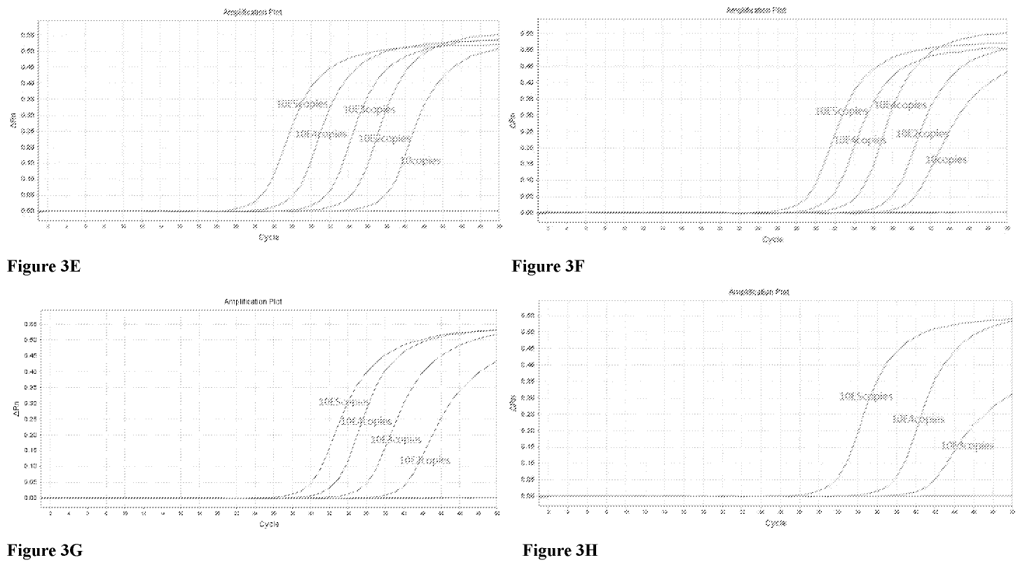

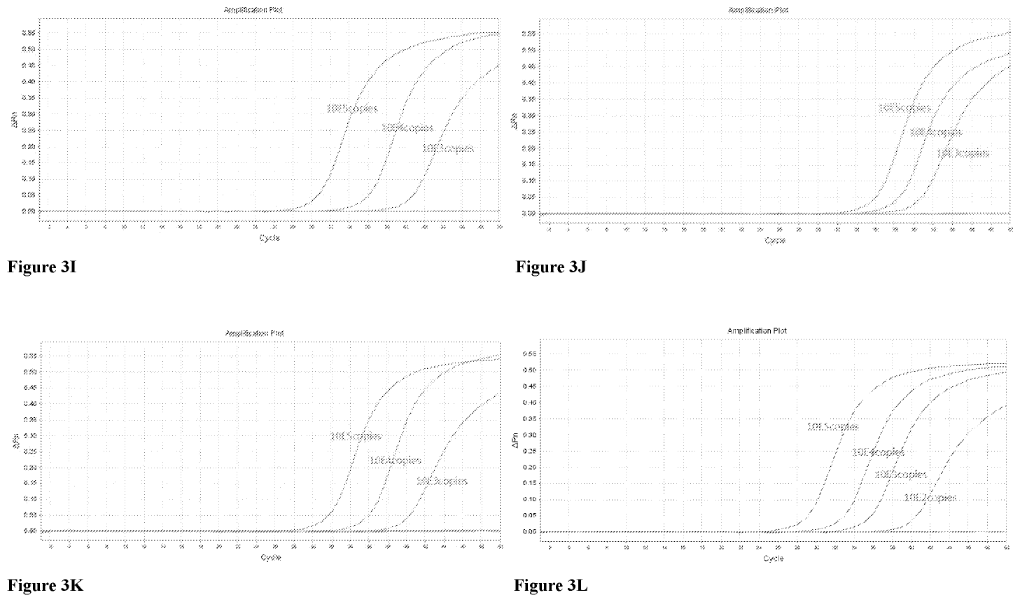

As shown in Table 13 and FIG. 3A to FIG. 3L, 1/10⁴ methylated DNA can be detected using the three pairs of preferred primers and probes, but three pairs of controls cannot detected.

As shown in Table 13 and FIG. 3A to FIG. 3L, the preferred three pairs of BMP3 and NDRG4 primers and probes of the present invention are capable of stably detecting methylation levels as low as one in ten thousand, while three pairs of comparative primers and probes are incapable of detecting one in ten thousand methylation levels.

TABLE 13
analytical sensitivity comparison of preferred and control primers and probes of BMP3 and NDRG4 genes.

| | | Copy | Copy | | | | | | |
|---|---|---|---|---|---|---|---|---|---|
| | | number of | number | Preferred primers and probes | | | Control primers and probes | | |
| | Methylated | methylated | of total | Preferred | Preferred | Preferred | Control | Control | Control |
| Gene | ratio | DNA | DNA | 1 | 2 | 3 | 1 | 2 | 3 |
| BMP3 | 1 | 10⁵ | 10⁵ | Y | Y | Y | Y | Y | Y |
| | 1/10 | 10⁴ | 10⁵ | Y | Y | Y | Y | Y | Y |
| | 1/10² | 10³ | 10⁵ | Y | Y | Y | Y | Y | Y |
| | 1/10³ | 10² | 10⁵ | Y | Y | Y | Y | N | N |
| | 1/10⁴ | 10 | 10⁵ | Y | Y | Y | N | N | N |
| NDRG4 | 1 | 10⁵ | 10⁵ | Y | Y | Y | Y | Y | Y |
| | 1/10 | 10⁴ | 10⁵ | Y | Y | Y | Y | Y | Y |
| | 1/10² | 10³ | 10⁵ | Y | Y | Y | Y | Y | Y |
| | 1/10³ | 10² | 10⁵ | Y | Y | Y | N | N | Y |
| | 1/10⁴ | 10 | 10⁵ | Y | Y | Y | N | N | N |

Y: detected,
N: Undetermined

Analytical sensitivity amplification curves of BMP3 and NDRG4 are shown in FIG. 3A to FIG. 3L for each of the preferred combination, compared to control groups.

| Primers and probes | BMP3 | NDRG4 |
|---|---|---|
| Preferred 1 | FIG. 3A | FIG. 3B |
| Preferred 2 | FIG. 3C | FIG. 3D |
| Preferred 3 | FIG. 3E | FIG. 3F |
| Control 1 | FIG. 3G | FIG. 3H |
| Control 2 | FIG. 3I | FIG. 3J |
| Control 3 | FIG. 3K | FIG. 3L |

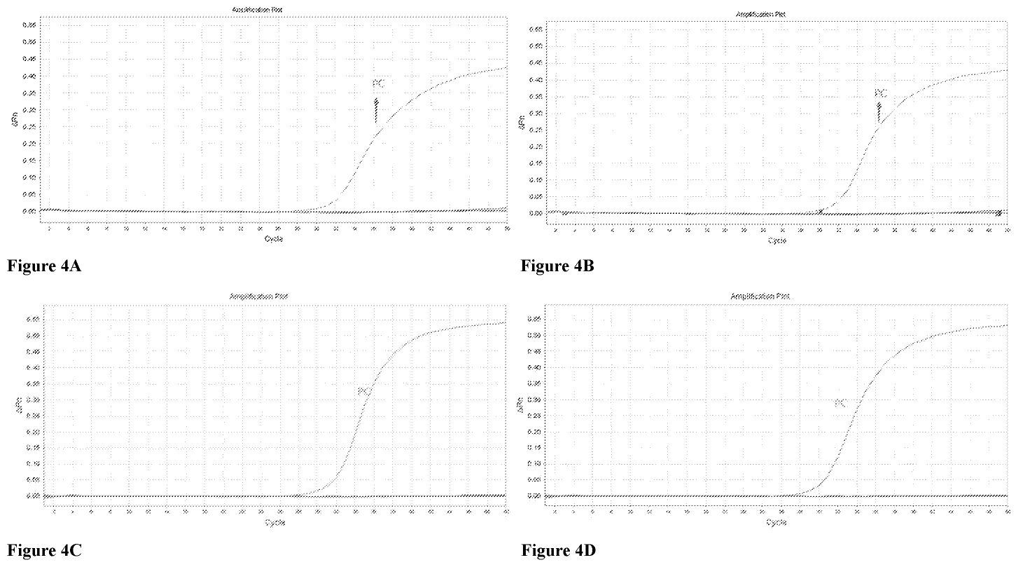

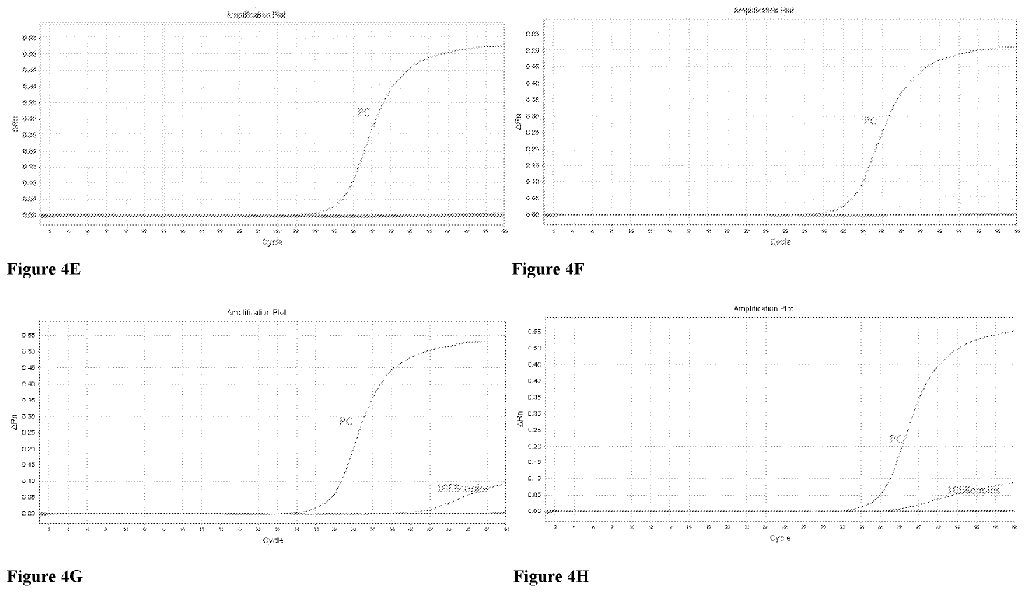

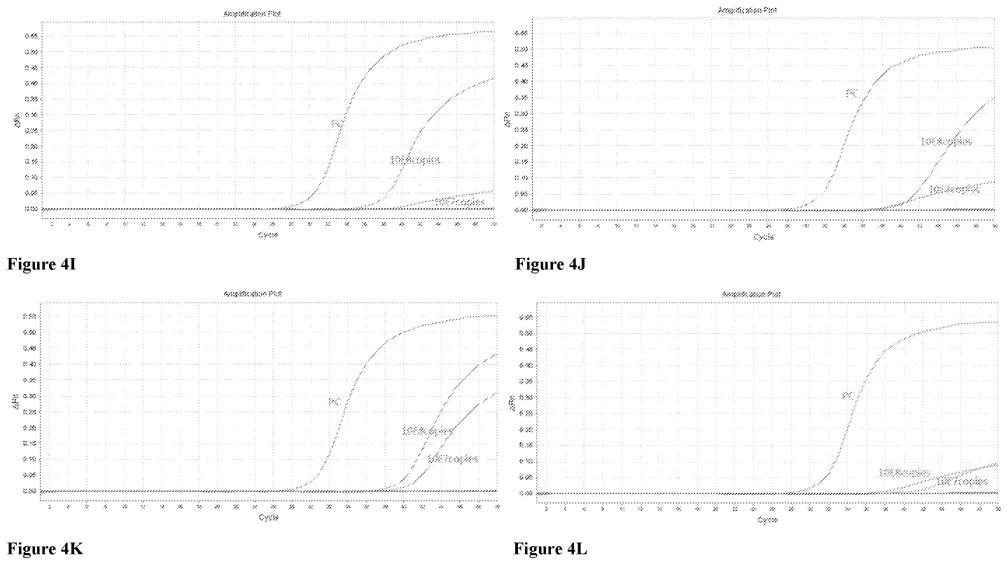

As shown in Table 14 and FIG. 4A to FIG. 4L, the three pairs of preferred primers and probes for BMP3 and NDRG4 genes have no amplification signals for different concentrations of unmethylated DNA, while the comparative primers and probes exhibit different degrees of non-specific amplification.

TABLE 14
Comparison of analytical specificity between
preferred and control primers and probe

| BMP3 | NDRG4 | | | | | | |
|---|---|---|---|---|---|---|---|
| (Copies | (Copies | Preferred primers and probes | | | Control primers and probes | | |
| per | per | Preferred | Preferred | Preferred | Control | Control | Control |
| reaction) | reaction) | 1 | 2 | 3 | 1 | 2 | 3 |
| 10⁸ | 10⁸ | N | N | N | Y | Y | Y |
| 10⁷ | 10⁷ | N | N | N | N | Y | Y |
| 10⁶ | 10⁶ | N | N | N | N | N | N |
| 10⁵ | 10⁵ | N | N | N | N | N | N |
| 10⁴ | 10⁴ | N | N | N | N | N | N |

Y: no amplification,
N: non-specific amplification

Analytical specificity amplification curves of BMP3 and NDRG4

| Primers and probes | BMP3 | NDRG4 |
|---|---|---|
| Preferred 1 | FIG. 4A | FIG. 4B |
| Preferred 2 | FIG. 4C | FIG. 4D |
| Preferred 3 | FIG. 4E | FIG. 4F |
| Control 1 | FIG. 4G | FIG. 4H |
| Control 2 | FIG. 4I | FIG. 4I |
| Control 3 | FIG. 4K | FIG. 4L |

### Example 4

Validation of Preferred and Comparative Methylated Primers and Probes is Performed with Fecal Samples of BMP3 and NDRG4 Genes.

Methylation level of BMP3 and NDRG4 genes in 81 fecal samples was detected with the three pairs of preferred and comparative primers and probes. The three comparative primers and probes are:

Comparative 1: BMP3 forward primer SEQ ID NO.:21, BMP3 reverse primer SEQ ID NO.: 22, and BMP3 probe SEQ ID NO.: 23; NDRG4 forward primer SEQ ID NO.: 24, NDRG4 reverse primer SEQ ID NO.: 25, and NDRG4 probe SEQ ID NO.: 26;
Comparative 2: BMP3 forward primer SEQ ID NO.:27, BMP3 reverse primer SEQ ID NO.: 28, and BMP3 probe SEQ ID NO.: 29, NDRG4 forward primer SEQ ID NO.: 24, NDRG4 reverse primer SEQ ID NO.: 30, and NDRG4 probe SEQ ID NO.: 26;
Comparative 3: BMP3 forward primer SEQ ID NO.:31, BMP3 reverse primer SEQ ID NO.: 32, and BMP3 probe SEQ ID NO.: 33; NDRG4 forward primer SEQ ID NO.: 24, NDRG4 reverse primer SEQ ID NO.: 34, and NDRG4 probe SEQ ID NO.: 26.

The information of fecal samples is showed in Table 15.

TABLE 15
the statistics of eighty-one fecal samples

| Colonoscopy | Numbers |
|---|---|
| Normal | 46 |
| adenomas | 15 |
| Colorectal cancer | 20 |
| total | 81 |

Fecal DNAs were extracted from samples by following the method described below.

### (1) Extraction of Fecal DNA

i. Add 40 mL of lysate to 4-6 g fecal samples, and vortex thoroughly, and then incubate at 50° C. for 16 hours.
ii. Then centrifuge at 5000 rpm for 10 minutes. Pay attention to the weighing balance before centrifugation. After the end of the centrifugation, carefully remove the centrifuge tubes and do not shake it vigorously.
iii. Pipette 9 mL of supernatant into anew 50 mL centrifuge tube, and then add 1 mL of extracting adjuvant, 60 μL of magnetic beads and 10 mL of isopropanol. Vortex for 10 seconds, and incubate at 65° C. for 20 minutes. Mix up and down once per 5 minutes during incubating.
iv. After the incubation, put the 50 mL centrifuge tube on a magnetic stand and keep steady for 3 minutes until the supernatant is clear and discard the supernatant.
v. Remove the 50 mL centrifuge tube from magnetic stand and add 12 mL of washing solution. Vortex until the magnetic beads fall off the tube well, and keep steady for 3 minutes, and put the 50 mL centrifuge tube back to magnetic stand for 3 minutes until the supernatant is clear and discarded.
vi. Add 15 mL of 80% ethanol solution, vortex until beads fall off the tube well and keep steady for 3 minutes. Put the tube back into magnetic stand and keep steady for 3 minutes until the supernatant is clear and discarded.
vii. Repeat the previous step once.
viii. Pipette the bottom residual liquid, keep the tube open, and incubate at 65° C. for 5 minutes. Remove the tube from the magnetic strand until the beads is dry and add 1.5 mL of preheated eluent I. Pipette the beads from the well to eluent I with 1000 μL pipette, and transfer the mixture to a 2 mL centrifuge tube and close the lid, and then incubate at 65° C. for 5 minutes.
ix. Centrifuge at 13000 rpm for 3 minutes, pipette 600 μL of supernatant to a new 1.5 mL of tube, and then add 600 μL of binding solution and vortex thoroughly.
x. Transfer 600 μL of the above mixture to a DNA purification column, centrifuge at 13,000 rpm for 1 minutes, and discard the waste.
xi. Deal with the remaining mixture as previous step, and centrifuge at 13,000 for 2 minutes.
xii. Add 600 μL of 90% ethanol solution to the DNA purification column, centrifuge at 13,000 rpm for 1 minute and discard the waste.
xiii. Repeat the previous step 2 times.
xiv. Centrifuge at 13,000 rpm for 3 minutes, and put the DNA purification column into a new 1.5 mL centrifuge tube. Open the DNA purification column and incubate at 65° C. for 5 minutes to dry.
xv. Drop 100 μL of preheated eluate II to the middle of the DNA purification column, close the lid, and then incubate at 65° C. for 5 minutes. Centrifuge at 13,000 rpm for 2 minutes. Obtain the eluted DNA solution and store it at 2-8° C. for use. Long-term storage should be kept at −25° C. to −15° C.

### (2) Bisulfite Treatment

The detailed operation steps according to example 3.

(3) qPCR

QPCR master mixture was prepared as follows:

TABLE 16

| NO. | Reagent | Final concentration | |
|---|---|---|---|
| 1 | 10 × PCR buffer | 1× | |
| 2 | MgCl₂ | 2 | mM |
| 3 | dNTP s | 0.2 | mM |
| 4 | Taq DNA Polymerase | 2 | U |
| 5 | Each primer | 0.75 | μM |
| 6 | Each probe | 0.25 | μM |
| 7 | DNA template | 2 | μL |

| | | |
|---|---|---|
| 8 | Ultrapure water | Up to 50 μL |

qPCR reaction condition is one cycle of denaturing at 95° C. for 2 minutes, and 50 cycles of denaturing at 95° C. for 20 seconds and elongating at 95° C. for 1 minutes. B2M gene is as a reference gene for quality control of qPCR reaction.

### (4) Results

As shown in Table 17, the sensitivity of CRC and AA detection in 81 fecal samples with the three pairs of preferred primers and probes of BMP3 and NDRG4 genes are up to 85.0° %-95.0% (CRC by BMP3 methylation), 66.7%-73.3% (AA by BMP3 methylation), and 90.0%-95.0% (CRC by NDRG4 methylation) and 73.3%-86.7% (AA by NDRG4 methylation), respectively. In addition, the specificity of CRC and AA detection using either BMP3 methylation data or NDRG4 methylation data is both about 97.8%-100.0%.

However, the sensitivity of CRC and AA detection in eighty-one fecal samples with the three pairs of comparative primers and probes of BMP3 and NDRG4 genes are 85.0%-90.0% % (CRC by BMP3 methylation), 46.7%-60.0% (AA by BMP3 methylation), and 90.0%-95.0% (CRC by NDRG4 methylation) and 66.7%-73.3% respectively (AA by NDRG4 methylation). Also, the overall specificity of CRC and AA detection using either BMP3 methylation data or NDRG4 methylation data is up about 91.3%-93.5% and 93.5%-95.7%, respectively.

It can be seen that the preferred primers and probes are superior to the comparative primers and probes in the detection of the clinical samples, especially for AA detection.

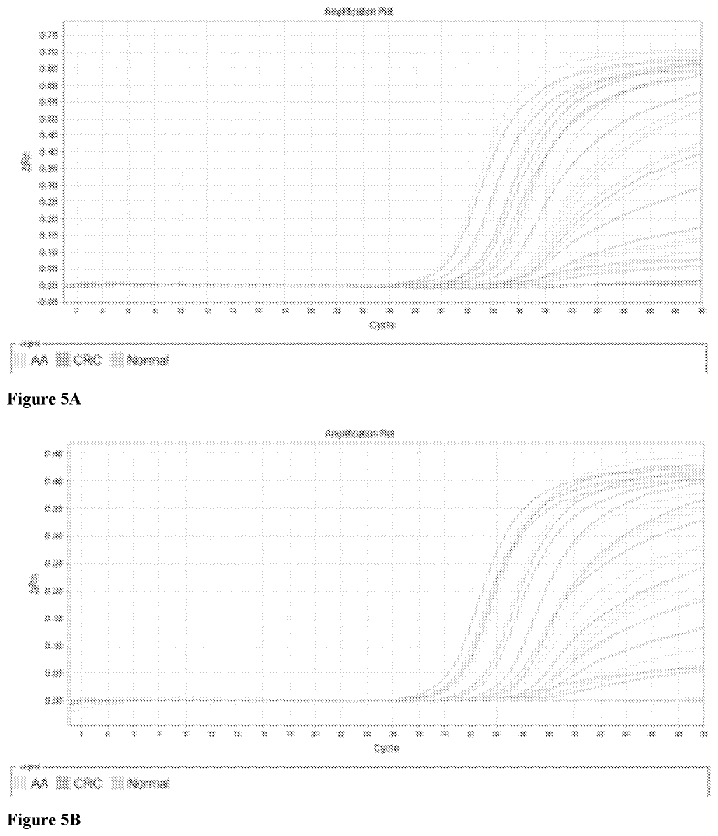

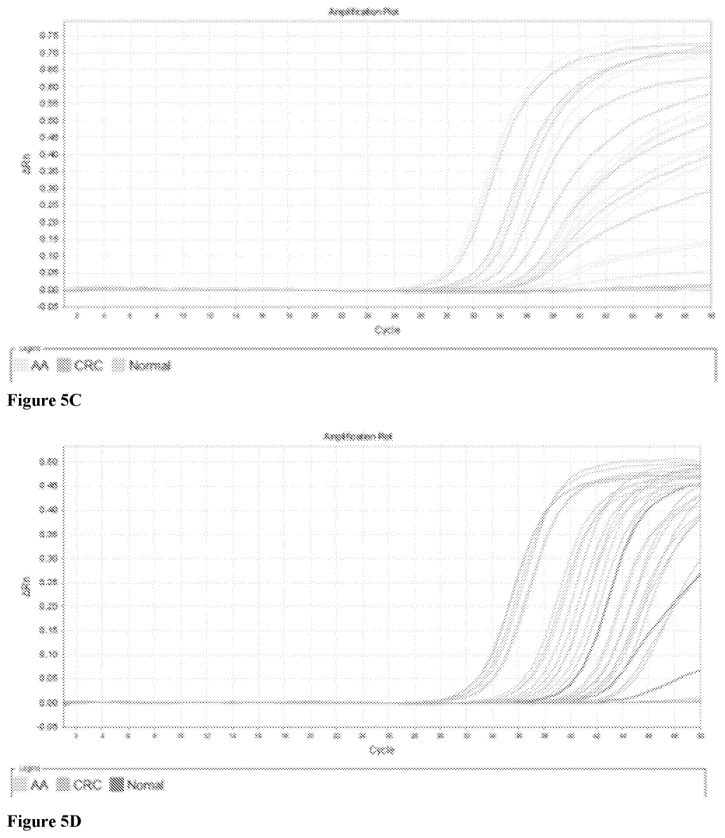

TABLE 17
the result statistics of clinical samples detection
BMP3

| Item | Preferred 1 | | | Preferred 2 | | | Preferred 3 | | |
|---|---|---|---|---|---|---|---|---|---|
| Samples grouping | CRC | AA | Normal | CRC | AA | Normal | CRC | AA | Normal |
| Number of positive samples | 19/20 | 11/15 | 0/46 | 18/20 | 11/15 | 1/46 | 17/20 | 10/15 | 1/46 |
| detected | | | | | | | | | |
| Sensitivity | 95.0% | 73.3% | / | 90.0% | 73.3% | / | 85.0% | 66.7% | / |
| Specificity | 100.0% | | | 97.8% | | | 97.8% | | |
| Total compliance rate | 93.8% | | | 91.4% | | | 88.9% | | |
| Amplification curve | FIG. 5A | | | FIG. 5B | | | FIG. 5C | | |
| Item | Comparatve 1 | | | Comparatve 2 | | | Comparatve 3 | | |
| Samples grouping | CRC | AA | Normal | CRC | AA | Normal | CRC | AA | Normal |
| Number of positive samples | 18/20 | 9/15 | 3/46 | 17/20 | 8/15 | 4/46 | 17/20 | 7/15 | 4/46 |
| detected | | | | | | | | | |
| Sensitivity | 90.0% | 60.0% | / | 85.0% | 53.3% | / | 85.0% | 46.7% | / |

| | | | |
|---|---|---|---|
| Specificity | 93.5% | 91.3% | 91.3% |
| Total compliance rate | 86.4% | 82.7% | 81.5% |
| Amplification curve | FIG. 5D | FIG. 5E | FIG. 5F |

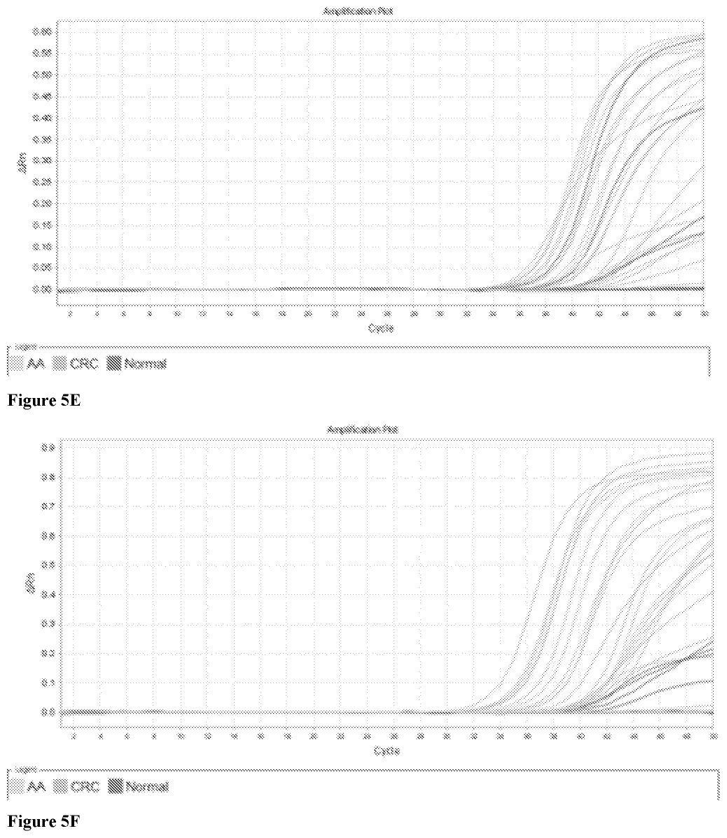

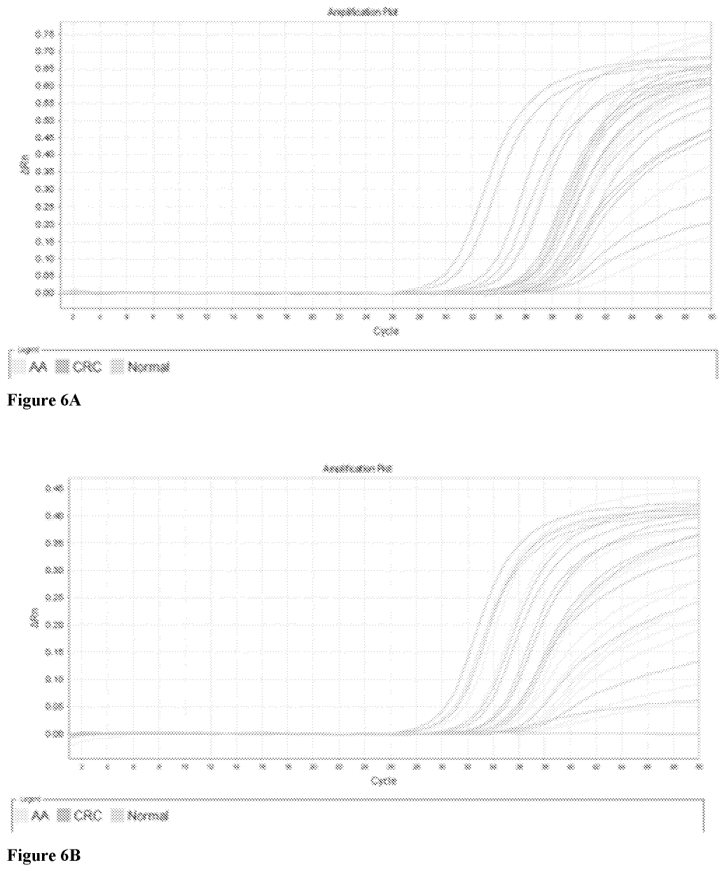

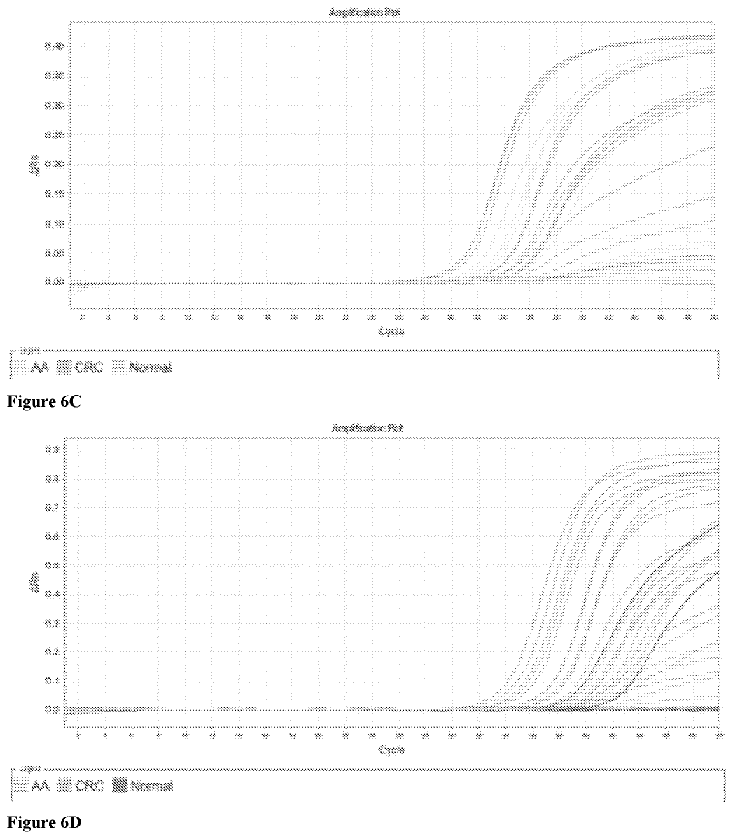

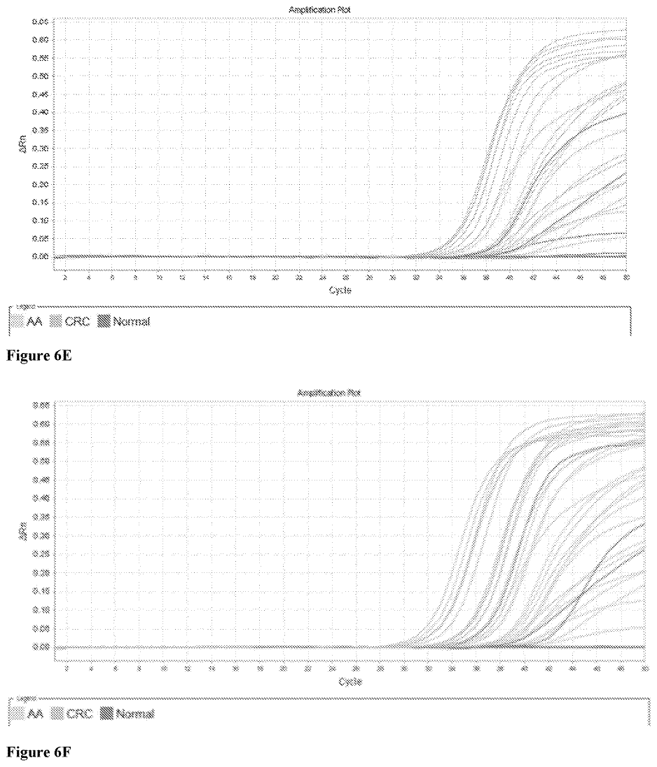

NDRG4

| Item | Preferred 1 | | | Preferred 2 | | | Preferred 3 | | |
|---|---|---|---|---|---|---|---|---|---|
| Samples grouping | CRC | AA | Normal | CRC | AA | Normal | CRC | AA | Normal |
| Number of positive samples | 19/20 | 13/15 | 0/46 | 19/20 | 12/15 | 0/46 | 18/20 | 11/15 | 1/46 |
| detected | | | | | | | | | |
| Sensitivity | 95.0% | 86.7% | / | 95.0% | 80.0% | / | 90.0% | 73.3% | / |
| Specificity | 100.0% | | | 100.0% | | | 97.8% | | |
| Total compliance rate | 96.3% | | | 95.1% | | | 91.4% | | |
| Amplification curve | FIG. 6A | | | FIG. 6B | | | FIG. 6C | | |
| Item | Comparatve 1 | | | Comparatve 2 | | | Comparatve 3 | | |
| Samples grouping | CRC | AA | Normal | CRC | AA | Normal | CRC | AA | Normal |
| Number of positive samples | 18/20 | 10/15 | 2/46 | 18/20 | 10/15 | 3/46 | 19/20 | 11/15 | 3/46 |
| detected | | | | | | | | | |
| Sensitivity | 90.0% | 66.7% | / | 90.0% | 66.7% | / | 95.0% | 73.3% | / |

| | | | |
|---|---|---|---|
| Specificity | 95.7% | 93.5% | 93.5% |
| Total compliance rate | 88.9% | 87.7% | 90.1% |
| Amplification curve | FIG. 6D | FIG. 6E | FIG. 6F |

TABLE 18
detection results of 81 fecal samples

| | | | | Ct BMP3 | | | | | | NDRG4 | | | | | |
|---|---|---|---|---|---|---|---|---|---|---|---|---|---|---|---|
| | | | | Preferred primers | | | Comparative primers | | | Preferred primers | | | Comparative primers | | |
| | | | | and probes | | | and probes | | | and probes | | | and probes | | |
| No. | Gender | Age | Colonoscopy | 1 | 2 | 3 | 1 | 2 | 3 | 1 | 2 | 3 | 1 | 2 | 3 |
| 1 | Man | 50 | CRC- stage I | 33.9 | 32.1 | 37.5 | 32.7 | 35.8 | 36.2 | U | 33.6 | 35 | 33.9 | 38.8 | 40.1 |
| 2 | Man | 51 | CRC-stage II | 34.9 | 32.2 | 29.1 | 41.3 | 37.6 | 39.9 | 39.4 | 38.4 | 33.8 | 34 | 38.1 | 40.3 |
| 3 | Female | 56 | CRC-stage III | 35.1 | 33.1 | 31.4 | 34.9 | 41.2 | 35.1 | 30.7 | 30.4 | 33.2 | 34.2 | 42.6 | 35 |
| 4 | Man | 72 | CRC-stage I | 35.7 | U | U | 33.4 | 40.1 | 36 | 37.2 | 38.9 | U | U | U | 36.7 |
| 5 | Female | 75 | CRC-stage II | 31.9 | 36.6 | 32.7 | 37.9 | 38.3 | 34.5 | 35.4 | 37.4 | 34.5 | 36.4 | 33 | 40.5 |
| 6 | Female | 55 | CRC-stage II | 37.5 | 35.1 | 29.5 | 43 | 39.8 | 39.7 | 34.4 | 35.9 | 35.6 | 35.1 | 37.4 | 42 |
| 7 | Female | 50 | CRC-stage II | 36.2 | 32.2 | 38.8 | 39.7 | 37.2 | 37.3 | 33.6 | 37.4 | 38.8 | 42.3 | 36.4 | 37.5 |
| 8 | Man | 62 | CRC-stage IV | U | U | U | U | U | U | 36.1 | 34.5 | 39.9 | 41 | 41.8 | 40.4 |
| 9 | Female | 74 | CRC-stage I | 35.7 | 37.7 | 29.3 | 38 | 36 | 44 | 29.6 | 32.3 | 36.6 | 42.6 | 36.8 | 33 |
| 10 | Female | 73 | CRC-stage II | 31.7 | 29.7 | 33.9 | 32.6 | 42 | 32.1 | 36.5 | 35.7 | 39.7 | 39.3 | 33.5 | 31.8 |
| 11 | Man | 52 | CRC-stage II | 34 | 40 | 35.9 | 39.5 | 38.1 | 42.4 | 36.9 | U | 36.5 | 42.6 | 37.8 | 36.6 |
| 12 | Female | 57 | CRC-stage II | 34.5 | 29.1 | 29.3 | 34.6 | 42.6 | 41.3 | 37.7 | 31.8 | 35.6 | 42.3 | 39 | 42.6 |
| 13 | Man | 51 | CRC-stage I | 37.6 | 34 | 33.2 | 38.8 | U | U | 31.9 | 35.4 | 35.9 | 39.1 | 35.1 | 34.6 |
| 14 | Man | 60 | CRC-stage II | 36.2 | 31.7 | 35.6 | 40.1 | 42.2 | 33.1 | 29.6 | 31.7 | 29.1 | 33.8 | 36.4 | 36.5 |
| 15 | Female | 49 | CRC-stage III | 31.1 | 37 | 29.4 | 41.9 | 35.8 | 34.3 | 34.4 | 33.6 | 35.6 | 40.3 | 39.2 | 39 |
| 16 | Man | 49 | CRC-stage IV | 34 | 35.5 | U | 41.8 | 35.9 | 37.5 | 37.6 | 32.3 | 32.3 | 38.1 | U | 31.3 |
| 17 | Man | 48 | CRC-stage I | 38.1 | 37.1 | 35.8 | 39 | 40.7 | 38.3 | 29.4 | 30.7 | U | U | 37.1 | U |
| 18 | Man | 54 | CRC-stage II | 36.1 | 33.7 | 35.2 | U | U | U | 29.4 | 37 | 33.5 | 34.7 | 34 | 41.5 |
| 19 | Female | 60 | CRC-stage III | 29.2 | 36.4 | 33.6 | 38.4 | 36.5 | 33.7 | 32.9 | 30.1 | 35.1 | 33.5 | 38.5 | 41.3 |
| 20 | Female | 62 | CRC-stage IV | 36.4 | 34.7 | 38.5 | 39.5 | 37.9 | 38.9 | 36.4 | 29.4 | 33.1 | 35.8 | 43.1 | 42.8 |
| 21 | Female | 50 | AA | 38.7 | U | U | 35.3 | U | U | 34.6 | 37 | U | U | U | 40.9 |
| 22 | Female | 56 | AA | U | 35.6 | 30 | 40.9 | 38.6 | 35 | 34.6 | U | 29.3 | 42.7 | 33.4 | U |
| 23 | Man | 58 | AA | U | U | 29.6 | 39.3 | 38 | 41.1 | 39.3 | 35.1 | 39.5 | 39.4 | U | 37.4 |
| 24 | Man | 67 | AA | 36.3 | 32.8 | 34.2 | U | U | U | 33.9 | 33 | 32.7 | 36 | 35.5 | U |
| 25 | Man | 51 | AA | 35.9 | 33.7 | 33.9 | 36.4 | 38 | 39 | 32.7 | 33.9 | U | U | 35.8 | 40.4 |
| 26 | Female | 71 | AA | 29.3 | 30 | 35.4 | 40.3 | 37.7 | 34.4 | 30.7 | 34.9 | 38.3 | 33.8 | 41.1 | 33 |
| 27 | Man | 65 | AA | U | 30.3 | U | U | U | U | 34.8 | 34 | 36.8 | 41.9 | 42.7 | 36.1 |
| 28 | Man | 62 | AA | 37.5 | 29.2 | U | U | U | U | U | U | 36.8 | 43.1 | 43.9 | 32.8 |
| 29 | Female | 45 | AA | 31 | 37.6 | 37.4 | 37.2 | 38.5 | 37.8 | 30.8 | 31.7 | 30.8 | U | U | U |
| 30 | Man | 67 | AA | 29.7 | 34.3 | 29.8 | 31.3 | U | U | 33.9 | U | U | U | U | U |
| 31 | Female | 53 | AA | 36.3 | U | U | U | 35.4 | 34.6 | 29.5 | 34.9 | 37.7 | 39.6 | 40 | 42.9 |
| 32 | Female | 68 | AA | 38.4 | 37.3 | 30.4 | U | U | U | U | 37.2 | U | U | U | 35.1 |
| 33 | Female | 50 | AA | 36.9 | 39 | 32.1 | 36.6 | U | U | 39.5 | 29.1 | 30.7 | 37.1 | 37.6 | 36.6 |
| 34 | Female | 48 | AA | U | U | U | U | 37.3 | 35.8 | 38.3 | 32.2 | 32.4 | 36.2 | 36 | 43.9 |
| 35 | Man | 50 | AA | 35.5 | 29.1 | 32.7 | 39.4 | 42.7 | U | 29.8 | 33.8 | 32.9 | 36.4 | 38.2 | 42 |
| 36 | Man | 45 | Normal | U | U | U | U | U | U | U | U | U | U | U | U |
| 37 | Female | 50 | Normal | U | U | U | U | U | U | U | U | U | U | U | U |
| 38 | Man | 59 | Normal | U | U | U | U | U | U | U | U | U | U | U | U |
| 39 | Man | 73 | Normal | U | U | U | U | U | U | U | U | U | U | U | U |
| 40 | Man | 47 | Normal | U | U | U | U | U | U | U | U | U | U | U | U |
| 41 | Man | 71 | Normal | U | U | U | U | U | U | U | U | U | U | U | U |
| 42 | Female | 45 | Normal | U | U | U | U | U | U | U | U | U | U | U | U |
| 43 | Female | 62 | Normal | U | U | U | U | U | U | U | U | U | U | U | U |
| 44 | Man | 55 | Normal | U | U | U | U | U | U | U | U | U | U | U | U |
| 45 | Female | 54 | Normal | U | U | U | U | U | U | U | U | U | U | U | U |
| 46 | Man | 63 | Normal | U | U | U | 39.4 | U | U | U | U | U | U | U | U |
| 47 | Female | 53 | Normal | U | U | U | U | U | U | U | U | U | 41.2 | U | U |
| 48 | Man | 67 | Normal | U | U | U | U | 37.2 | 41.2 | U | U | U | U | U | U |
| 49 | Man | 69 | Normal | U | U | U | U | U | U | U | U | U | U | U | U |
| 50 | Man | 54 | Normal | U | U | U | U | U | U | U | U | U | U | U | U |
| 51 | Man | 50 | Normal | U | U | U | U | U | U | U | U | U | U | U | U |
| 52 | Female | 65 | Normal | U | U | U | U | U | U | U | U | U | U | 40.3 | 41 |
| 53 | Female | 56 | Normal | U | U | U | U | U | U | U | U | U | U | U | U |
| 54 | Man | 67 | Normal | U | 35.6 | U | U | 41.3 | U | U | U | U | U | U | U |
| 55 | Man | 70 | Normal | U | U | U | U | U | U | U | U | U | U | U | U |
| 56 | Female | 75 | Normal | U | U | U | U | U | U | U | U | U | U | U | U |
| 57 | Female | 50 | Normal | U | U | U | U | U | U | U | U | U | U | U | U |
| 58 | Female | 66 | Normal | U | U | U | U | U | U | U | U | U | U | U | U |
| 59 | Man | 49 | Normal | U | U | U | U | U | 41.6 | U | U | U | U | U | U |
| 60 | Man | 71 | Normal | U | U | U | 40.8 | U | U | U | U | U | U | U | U |
| 61 | Man | 70 | Normal | U | U | U | U | U | U | U | U | U | U | 40.5 | U |
| 62 | Man | 73 | Normal | U | U | U | U | U | U | U | U | U | U | U | U |
| 63 | Man | 48 | Normal | U | U | U | U | 38.5 | U | U | U | U | U | U | U |
| 64 | Man | 52 | Normal | U | U | U | U | U | U | U | U | U | U | U | 39.6 |
| 65 | Man | 71 | Normal | U | U | U | U | U | U | U | U | U | U | U | U |
| 66 | Female | 60 | Normal | U | U | U | U | U | 41.9 | U | U | 35.9 | 39.4 | U | U |
| 67 | Female | 65 | Normal | U | U | U | U | U | U | U | U | U | U | U | U |
| 68 | Female | 74 | Normal | U | U | U | U | U | U | U | U | U | U | U | U |
| 69 | Man | 71 | Normal | U | U | U | U | U | U | U | U | U | U | U | U |
| 70 | Female | 73 | Normal | U | U | U | U | U | U | U | U | U | U | U | U |
| 71 | Man | 49 | Normal | U | U | U | U | U | U | U | U | U | U | U | U |
| 72 | Female | 69 | Normal | U | U | U | U | U | U | U | U | U | U | U | U |
| 73 | Man | 59 | Normal | U | U | U | 42.7 | 41.9 | 42.5 | U | U | U | U | U | U |
| 74 | Man | 71 | Normal | U | U | 37.1 | U | U | U | U | U | U | U | U | U |
| 75 | Female | 54 | Normal | U | U | U | U | U | U | U | U | U | U | 38.9 | 35.2 |
| 76 | Man | 71 | Normal | U | U | U | U | U | U | U | U | U | U | U | U |
| 77 | Female | 63 | Normal | U | U | U | U | U | U | U | U | U | U | U | U |
| 78 | Man | 74 | Normal | U | U | U | U | U | U | U | U | 11 | U | U | U |
| 79 | Man | 62 | Normal | U | U | U | U | U | U | U | U | U | U | U | U |
| 80 | Female | 75 | Normal | U | U | U | U | U | U | U | U | U | U | U | U |
| 81 | Man | 48 | Normal | U | U | U | U | U | U | U | U | U | U | U | U |

Notes
U: undetermined

### Example 5

### Screening of Methylation Primer and Probe Combinations of BMP3 and DNRG4 Genes

### (1) Fecal DNA Extraction

Fecal DNA is extracted according to the protocol of example 3.

### (2) Methylation Detection of BMP3 and NDRG4 Genes

The combinations of primer and probe of BMP3 and NDRG4 genes is shown as follow. qPCR was performed with the nine combinations respectively according to the steps of example 3.

TABLE 19
Combinations of primer and probe

| Combination No. | BMP3 | NDRG4 |
|---|---|---|
| 1 | Preferred 3 | Preferred 1 |
| 2 | Preferred 3 | Preferred 2 |
| 3 | Preferred 3 | Preferred 3 |
| 4 | Preferred 1 | Preferred 1 |
| 5 | Preferred 1 | Preferred 2 |
| 6 | Preferred 1 | Preferred 3 |
| 7 | Preferred 2 | Preferred 1 |
| 8 | Preferred 2 | Preferred 2 |
| 9 | Preferred 2 | Preferred 3 |

The sequences of primers and probes of nine combinations of BMP3 and NDRG4 genes are as follows:

TABLE 20
Sequences of primers and probes in the combinations

| Combination | | | | | Sequence |
|---|---|---|---|---|---|
| No. | Gene | Group | Printer/Probe | Sequence ID | (5′ to 3′) |
| 1 | BMP3 | Preferred 3 | Forward primer | SEQ ID NO.: 15 | AATATTCGGGTTATATACGTCGCGATT |
| | | | Reverse primer | SEQ ID NO.: 16 | ACTTACTACGCTAACCCAACG |
| | | | Probe | SEQ ID NO.: 17 | TAGCGTTGGAGTGGAGACGGCGTTCGTA |
| | NDRG4 | Preferred 1 | Forward primer | SEQ ID NO.: 6 | ATCGATCGGGGTCTTTTTTAGGTTTC |
| | | | Reverse primer | SEQ ID NO.: 7 | CCTTCTACGCGACTAAAATACCCGAT |
| | | | Probe | SEQ ID NO.: 8 | CGTCGCGGTTTTCGTTCGTTTTTTCGTTCGT |
| 2 | BMP3 | Preferred 3 | Forward primer | SEQ ID NO.: 15 | AATATTCGGGTTATATACGTCGCGATT |
| | | | Reverse pnmer | SEQ ID NO.: 16 | ACTTACTACGCTAACCCAACG |
| | | | Probe | SEQ ID NO.: 17 | TAGCGTTGGAGTGGAGACGGCGTTCGTA |
| | NDRG4 | Preferred 2 | Forward primer | SEQ ID NO.: 12 | GCGGGTGAGAAGTCGGC |
| | | | Reverse primer | SEQ ID NO.: 13 | GTAACTTCCGCCTTCTACGC |
| | | | Probe | SEQ ID NO.: 14 | TAGGTTTCGCGTCGCGGTTTTCGTT |
| 3 | BMP3 | Prefened 3 | Forward primer | SEQ ID NO.: 15 | AATATTCGGGTTATATACGTCGCGATT |
| | | | Reverse primer | SEQ ID NO.: 16 | ACTTACTACGCTAACCCAACG |
| | | | Probe | SEQ ID NO.: 17 | TAGCGTTGGAGTGGAGACGGCGTTCGTA |
| | NDRG4 | Preferred 3 | Forward primer | SEQ ID NO.: 18 | CGGTTTTCGTTCGTTTTTTCG |
| | | | Reverse primer | SEQ ID NO.: 19 | AACCTAAAACTAATCCCGAACGAACC |
| | | | Probe | SEQ ID NO.: 20 | TCGTTTATCGGGTATTTTAGTCGCGTAG |
| 4 | BMP3 | Preferred 1 | Forward primer | SEQ ID NO.: 3 | TTTGAAAATATTCGGGTTATATACGTCGC |
| | | | Reverse primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred ! | Forward primer | SEQ ID NO.: 6 | ATCGATCGGGGTGTTTTTTAGGTTTC |
| | | | Reverse primer | SEQ ID NO.: | CCTTCTACGCGACTAAAATACCCGAT |
| | | | Probe | SEQ ID NO.: 8 | CGTCGCGGTTTTCGTTCGTTTTTTCGTTCGT |
| 5 | BMP3 | Preferred 1 | Forward primer | SEQ ID NO.: 3 | TTTGAAAATATTCGGGTTATATACGTCGC |
| | | | Reverse primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred 2 | Forward primer | SEQ ID NO.: 12 | GCGGGTGAGAAGTCGGC |
| | | | Reverse primer | SEQ ID NO.: 13 | GTAACTTCCGCCTTCTACGC |
| | | | Probe | SEQ ID NO.: 14 | TAGGTTTCGCGTCGCGGTTTTCGTT |
| 6 | BMP3 | Preferred 1 | Forward primer | SEQ ID NO.: 3 | TTTGAAAATATTCGGGTTATATACGTCGC |
| | | | Reverse primer | SEQ ID NO.: 4 | ATAAACTCTTCCCCAACAACTACGCGAA |
| | | | Probe | SEQ ID NO.: 5 | AGCGTTGGAGTGGAGACGGCGTTCG |
| | NDRG4 | Preferred 3 | Forward primer | SEQ ID NO.: 18 | CGGTTTTCGTTCGTTTTTTCG |
| | | | Reverse primer | SEQ ID NO.: 19 | AACCTAAAACTAATCCCGAACGAACC |
| | | | Probe | SEQ ID NO.: 20 | TCGTTTATCGGGTATTTTAGTCGCGTAG |
| 7 | BMP3 | Preferred 2 | Forward primer | SEQ ID NO.: 9 | AATATTCGGGTTATATACGTCGCGA |
| | | | Reverse punier | SEQ ID NO.: 10 | GCAACCTAACAAATAAACTCTTCCCCAA |
| | | | Probe | SEQ ID NO.: 11 | TGGAGTGGAGACGGCGTTCGTAGCGT |
| | NDRG4 | Preferred 1 | Forward primer | SEQ ID NO.: 6 | ATCGATCGGGGTGTTTTTTAGGTTTC |
| | | | Reverse primer | SEQ ID NO.: 7 | CCTTCTACGCGACTAAAATACCCGAT |
| | | | Probe- | SEQ ID NO.: 8 | CGTCGCGGTTTTCGTTCGTTTnTCGTTCGT |
| 8 | BMP3 | Preferred 2 | Forward primer | SEQ ID NO.: 9 | AATATTCGGGTTATATACGTCGCGA |
| | | | Reverse primer | SEQ ID NO.: 10 | GCAACCTAACAAATAAACTCTTCCCCAA |
| | | | Probe | SEQ ID NO.: 11 | TGGAGTGGAGACGGCGTTCGTAGCGT |
| | NDRG4 | Preferred 2 | Forward primer | SEQ ID NO.: 12 | GCGGGTGAGAAGTCGGC |
| | | | Reverse primer | SEQ ID NO.: 13 | GTAACTTCCGCCTTCTACGC |
| | | | Probe | SEQ ID NO.: 14 | TAGGTTTCGCGTCGCGGTTTTCGTT |
| 9 | BMP3 | Preferred 2 | Forward primer | SEQ ID NO.: 9 | AATATTCGGGTTATATACGTCGCGA |
| | | | Reverse primer | SEQ ID NO.: 10 | GCAACCTAACAAATAAACTCTTCCCCAA |
| | | | Probe | SEQ ID NO.: 11 | TGGAGTGGAGACGGCGTTCGTAGCGT |
| | NDRG4 | Preferred 3 | Forward primer | SEQ ID NO.: 18 | CGGTTTTCGTTCGTTTTTTCG |
| | | | Reverse primer | SEQ ID NO.: 19 | AACCTAAAACTAATCCCGAACGAACC |
| | | | Probe | SEQ ID NO.: 20 | TCGTTTATCGGGTATTTTAGTCGCGTAG |

QPCR master mixture used in the reaction is as follows:

TABLE 21
QPCR master mixture for BMP3/NDRG4

| NO. | Reagents | Final concentration | |
|---|---|---|---|
| 1 | 10 × PCR buffer | 1× | |
| 2 | MgCl₂ | 2 | mM |
| 3 | dNTP s | 0.2 | mM |
| 4 | Taq DNA Polymerase | 2 | U |
| 5 | Each primer | 0.75 | μM |
| 6 | Each probe | 0.25 | μM |
| 7 | DNA template | 2 | μL |

| | | |
|---|---|---|
| 8 | Ultrapure water | Up to 50 μL |

The qPCR reaction condition is one cycle of denatured at 95° C. for 2 minutes, and 50 cycles of denatured at 95° C. for 20 seconds and elongated at 60° C. for 1 minute.

### (3) Variants Detection of KRAS Gene

Seven mutation hotspots in codon 12 and 13 of KRAS gene were detected. The seven mutant are G12D [symbol] G13D [symbol] G12V [symbol] G12C [symbol] G12S [symbol] G12A and G13R and sequences of which primers and probes are in Table 22.

Table 22
Primers and probes to detect
seven mutation of KRAS gene

| Primer | Primer/ | Sequence | Sequence |
|---|---|---|---|
| Name | Probe | ID | (5' to 3') |
| G12D-F | Forward | SEQ ID | AACTTGTGGTA |
| | primer | NO.: 35 | GTTGGAGGTGA |
| G13D-F | Forward | SEQ ID | AACTTGTGGT |
| | primer | NO.: 36 | AGTTGGAGCT |
| | | | GGGGA |
| G12V-F | Forward | SEQ ID | AACTTGTGGT |
| | primer | NO.: 37 | AGTTGGAGTT |
| | | | GT |
| G12C-F | Forward | SEQ ID | AAACTTGTGG |
| | primer | NO.: 38 | TAGTTGGGGC |
| | | | TT |
| G12S-F | Forward | SEQ ID | AAACTTGTGG |
| | primer | NO.: 39 | TAGTTGGTGC |
| | | | TA |
| G12A-F | Forward | SEQ ID | AACTTGTGGT |
| | primer | NO.: 40 | AGTTGGAGCA |
| | | | GC |
| G12R-F | Forward | SEQ ID | AAACTTGTGGT |
| | primer | NO.: 41 | AGTTGGAGCTC |
| Kras-R | Reverse | SEQ ID | GAATGGTCCTG |
| | primer | NO.: 42 | CACCACTAATA |
| | | | TG |
| ACTB-F | Forward | SEQ ID | AGGGCTTCTTG |
| | primer | NO.: 43 | TCCTTTCCTT |
| ACTB-R | Reverse | SEQ ID | CGTGCTCGATG |
| | primer | NO.: 44 | GGGTACTTC |
| KRAS-P | Probe | SEQ ID | AGGCAAGAGTG |
| | | NO.: 45 | CCTTGACGATA |
| | | | CAGC |
| ACTB-P | Probe | SEQ ID | CGTGATGGTGG |
| | | NO.: 46 | GCATGGGTCAG |
| | | | AAGGA |

### QPCR Master Mixture Used in the Reaction is as Follows:

TABLE 23
QPCR master mixture for KRAS

| No. | Reagents | Final concntration | |
|---|---|---|---|
| 1 | G12A-F | 0.72 | μM |
| 2 | G12C-F | 0.60 | μM |
| 3 | G12D-F | 0.72 | μM |
| 4 | G12R-F | 0.48 | μM |
| 5 | G12S-F | 0.90 | μM |
| 6 | G12V-F | 0.72 | μM |
| 7 | G13D-F | 0.48 | μM |
| 8 | Kras-R | 0.90 | μM |
| 9 | Kras-P | 0.10 | μM |
| 10 | ACTB-F | 0.30 | μM |
| 11 | ACTB -R | 0.30 | μM |
| 12 | ACTB -P | 0.05 | μM |
| 13 | 5 × PCR Buffer, —Mg²⁺ | 1× | |
| 14 | MgCl₂ | 1.0 | mM |
| 15 | dNTPs | 0.1 | mM |
| 16 | Taq DNA Polymerase | 2.5 | U |
| 17 | DNA template | 2 | μL |

| | | |
|---|---|---|
| 18 | Ultrapure water | Up to 50 μL |

QPCR reaction condition is one cycle of denatured at 95° C. for 5 minutes and 45 cycles of denatured at 95° C. for 15 seconds, annealed at 71° C. for 60 seconds and then elongated at 55° C. for 50 seconds.

The reaction quality control of qPCR was carried out using ACTB as a reference gene.

### (4) Fecal Hemoglobin Test

Fecal hemoglobin was detected with fecal immunochemical Test (FIT), and the result is positive or negative.

(5) Generate a Score with a Formula

Bring the Ct value of qPCR detection of BMP3, NDRG4 and KRAS genes, and positive and negative result of fecal hemoglobin test into the logistic regression formula as follow:

P=e^{K}/(1+e^{K})

Where: P is a comprehensive index, K=a*ΔCt1+b*ΔCt2+c*ΔCt3+d*FIT+X, e is a natural constant, a, b, c, d, X are clinical constant. ΔCt1, ΔCt2, and ΔCt3 are Ct value of target genes subtract to that of reference genes.

The test result is positive if P value is equal or more than a predetermined threshold, otherwise is negative. Positive result indicates that the subject is possible to have CRC or AA.

### (6) Test Result

The eighty-one fecal samples of example 3 were detected and the results of different combinations of primers and probes of BMP3 and NDRG4 genes are shown in Table 24.

TABLE 24
the detection result of 81 fecal samples

| | | | | | | | Number of | | |
|---|---|---|---|---|---|---|---|---|---|
| | | | | | | | samples | | |
| | | | | | Number of | Number of | tested | | |
| Combination | | | Sensitivity | Specificity | samples tested | samples tested | negative in | Sensitivity | Sensitivity |
| No. | BMP3 | NDRG4 | (CRC + AA) | (CRC + AA) | positive in CRC | positive in AA | normal | (CRC) | (AA) |
| 1 | Preferred 3 | Preferred 1 | 94.3% | 97.8% | 19/20 | 14/15 | 45/46 | 95.0% | 93.3% |
| 2 | Preferred 3 | Preferred 2 | 91.4% | 97.8% | 18/20 | 14/15 | 45/46 | 90.0% | 93.3% |
| 3 | Preferred 3 | Preferred 3 | 80.0% | 95.6% | 16/20 | 12/15 | 44/46 | 80.0% | 80.0% |
| 4 | Preferred 1 | Preferred 1 | 97.1% | 100.0% | 20/20 | 14/15 | 46/46 | 100.0% | 93.3% |
| 5 | Preferred 1 | Preferred 2 | 97.1% | 97.8% | 20/20 | 14/15 | 45/46 | 100.0% | 93.3% |
| 6 | Preferred 1 | Preferred 3 | 88.6% | 95.6% | 19/20 | 12/15 | 44/46 | 95.0% | 80.0% |
| 7 | Preferred 2 | Preferred 1 | 97.1% | 97.8% | 19/20 | 15/15 | 45/46 | 95.0% | 100.0% |
| 8 | Preferred 2 | Preferred 2 | 94.3% | 97.8% | 19/20 | 14/15 | 45/46 | 95.0% | 93.3% |
| 9 | Preferred 2 | Preferred 3 | 85.7% | 95.6% | 19/20 | 11/15 | 44/46 | 95.0% | 73.3% |

It can be seen that: (1) Combination Nos. 4, 5 and 7 are superior to other six combinations. Considering all tested primers and probes are preferred ones and are superior to other primers and probes (see Example 3), the three particular combinations are superior to any known combinations of BMP3 and NDRG4 primers/probes. (2) The sensitivity and specificity of the kit for CRC and AA detection comprising BMP3, NDRG4, KRAS genes and fecal hemoglobin detection is significantly better than those of BMP3 or NDRG4 single gene methylation detection. (3) The sensitivity and specificity of the kit for CRC detection in the Asian population (e.g., the Chinese population) is obviously superior to existing similar products, such as Cologuard®. (4) The sensitivity and specificity of the kit for AA detection in the Asian population (e.g., the Chinese population) is significantly better than the existing similar products, such as Cologuard®.

The disclosures, including the claims, figures and/or drawings, of each and every patent, patent application, and publication cited herein are hereby incorporated herein by reference in their entireties.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A kit for detecting the presence or the absence of colorectal cancer (CRC) or advanced adenoma (AA) in a patient in need thereof, comprising:
a) a first pair of primers and a first probe for detecting the methylation state or level of at least one CpG dinucleotide of the BMP3 gene in a biological sample obtained from the patient, wherein the first pair of primers and first probe, each of which comprises a contiguous sequence of at least 16 nucleotides that is identical to, complementary to, or hybridizes under stringent hybridization conditions to SEQ ID NO.: 1,
b) a second pair of primers and a second probe for detecting the methylation state or level of at least one CpG dinucleotide of the NDRG4 gene in a biological sample obtained from the patient, wherein the second pair of primers and second probe, each of which comprises a contiguous sequence of at least 16 nucleotides that is identical to, complementary to, or hybridizes under stringent hybridization conditions to SEQ ID NO.: 2,

2. The kit of claim 1,
wherein the first pair of primers and the first probe are selected from the group consisting of:
i) a forward primer comprising SEQ ID NO.: 3, a reverse primer comprising SEQ ID NO.: 4, and a probe comprising SEQ ID NO.: 5;
ii) a forward primer comprising SEQ ID NO.: 9, a reverse primer comprising SEQ ID NO.: 10, and a probe comprising SEQ ID NO.: 11; and
iii) a forward primer comprising SEQ ID NO.: 15, a reverse primer comprising SEQ ID NO.: 16, and a probe comprising SEQ ID NO.: 17;
and,
wherein the second first pair of primers and the second probe are selected from the group consisting of:
iv) a forward primer comprising SEQ ID NO.: 6, a reverse primer comprising SEQ ID NO.: 7, and a probe comprising SEQ ID NO.: 8;
v) a forward primer comprising SEQ ID NO.: 12, a reverse primer comprising SEQ ID NO.: 13, and a probe comprising SEQ ID NO.: 14; and
vi) a forward primer comprising SEQ ID NO.: 18, a reverse primer comprising SEQ ID NO.: 19, and a probe comprising SEQ ID NO.: 20;

3. The kit of claim 1, wherein the kit comprises:
i) a forward primer comprising SEQ ID NO.: 3, a reverse primer comprising SEQ ID NO.: 4, and a probe comprising SEQ ID NO.: 5, for detecting the methylation state or level of at least one CpG dinucleotide of the BMP3 gene in the biological sample obtained from the patient, and
ii) a forward primer comprising SEQ ID NO.: 6, a reverse primer comprising SEQ ID NO.: 7, and a probe comprising SEQ ID NO.: 8, for detecting the methylation state or level of at least one CpG dinucleotide of the NDRG4 gene in the biological sample obtained from the patient.

4. The kit of claim 1, wherein the kit comprises:
i) a forward primer comprising SEQ ID NO.: 9, a reverse primer comprising SEQ ID NO.: 10, and a probe comprising SEQ ID NO.: 11, for detecting the methylation state or level of at least one CpG dinucleotide of the BMP3 gene in the biological sample obtained from the patient, and
ii) a forward primer comprising SEQ ID NO.: 12, a reverse primer comprising SEQ ID NO.: 13, and a probe comprising SEQ ID NO.: 14, for detecting the methylation state or level of at least one CpG dinucleotide of the NDRG4 gene in the biological sample obtained from the patient.

5. The kit of claim 1, wherein the kit comprises:
i) a forward primer comprising SEQ ID NO.: 15, a reverse primer comprising SEQ ID NO.: 16, and a probe comprising SEQ ID NO.: 17, for detecting the methylation state or level of at least one CpG dinucleotide of the BMP3 gene in the biological sample obtained from the patient, and
ii) a forward primer comprising SEQ ID NO.: 18, a reverse primer comprising SEQ ID NO.: 19, and a probe comprising SEQ ID NO.: 20, for detecting the methylation state or level of at least one CpG dinucleotide of the NDRG4 gene in the biological sample obtained from the patient.

6. The kit of any one of claims 1 to 5, wherein both the first probe and the second probe comprise a fluorescent donor and an acceptor fluorophore.

7. The kit of claim 6, wherein first probe and the second probe are TAQMAN® probes.

8. The kit of any one of claims 1 to 7, wherein the kit further comprises:
(1) means for detecting the presence or absence of at least one mutation in the KRAS gene in the patient; and
(2) means for detecting the presence or absence of hemoglobin in a biological sample obtained from the patient.

9. The kit of claim 8, wherein the means for detecting the presence or absence of at least one mutation in the KRAS gene in the patient comprises at least one pair of primers capable of amplifying the Exon 12 and/or Exon 13 region of the KRAS gene in a polymerase chain reaction (PCR).

10. The kit of claim 8, wherein the means for detecting the presence or absence of hemoglobin in the biological sample comprises an anti-hemoglobin antibody.

11. The kit of claim 9, wherein the primers are capable of amplifying a KRAS gene region comprising at least one KRAS mutation selected from the group consisting G12D, G12V, G12C, G13D, G12A, G12R, G12S, and G13C.

12. The kit of claim 10, wherein the antibody is a colloidal gold-conjugated antibody.

13. The kit of any one of claims 1 to 12, wherein the kit further comprises means for amplifying an a reference gene for quantification.

14. The kit of any one of claims 1 to 12, wherein the kit further comprises instructions for use and/or interpretation of a test result obtained by using the kit.

15. The kit of claim 10, wherein the kit further comprises means to detect a complex formed by the antibody and the hemoglobin in the biological sample.

16. The kit of any one of claims 1 to 15, wherein the biological sample obtained from the patient is a fecal sample.

17. The kit of any one of claims 1 to 16, wherein the kit further comprises a bisulfite reagent, and a container suitable for mixing the bisulfite reagent and the biological sample of the patient, or polynucleotides obtained from the biological sample.

18. The kit of any one of claims 1 to 17, wherein the kit further comprises a methylation sensitive restriction enzyme reagent.

19. The kit of any one of claims 1 to 18, wherein the kit further comprises
(1) a positive standard and a negative standard for detecting BMP3 methylation in the biological sample, and
(2) a positive standard and a negative standard for detecting NDRG4 methylation in the biological sample.

20. The kit of claim 19, wherein
the positive standard for detecting BMP3 methylation comprises a polynucleotide sequence of
(SEQ ID NO: 67)
GTTAGTTTGGTCGGGTGTTTTTAAAAATAAAGCGAGGAGGGAAG
GTATAGATAGATTTTGAAAATATTCGGGTTATATACGTCGCGAT
TTATAGTTTTTTTTTAGCGTTGGAGTGGAGACGGCGTTCGTAGC
GTTTTGCGCGGGTGAGGTTCGCGTAGTTGTTGGGGAAGAGTTTA
TTTGTTAGGTTGCGTTGGGTTAGCGTAGTAAGTGGGGTTGGTCG
TTATTTCGTTGTATTCGGTCGCGTTTCGGGTTTCGTGCGTTTTC
GTTTTAG;
the negative standard for detecting BMP3 methylation comprises a polynucleotide sequence of
(SEQ ID NO: 68)
GTTAGTTTGGTTGGGTGTTTTTAAAAATAAAGTGAGGAGGGAAG
GTATAGATAGATTTTGAAAATATTTGGGTTATATATGTTGTGAT
TTATAGTTTTTTTTTAGTGTTGGAGTGGAGATGGTGTTTGTAGT
GTTTTGTGTGGGTGAGGTTTGTGTAGTTGTTGGGGAAGAGTTTA
TTTGTTAGGTTGTGTTGGGTTAGTGTAGTAAGTGGGGTTGGTTG
TTATTTGTTGTATTTGGTTGTGTTTTGGGTTTTGTGTGTTTTTG
TTTTAG;
the positive standard for detecting NDRG4 methylation comprises a polynucleotide sequence of
(SEQ ID NO: 69)
TGAGAAGTCGGCGGGGGCGCGGATCGATCGGGGTGTTTTTTAGG
TTTCGCGTCGCGGTTTTCGTTCGTTTTTTCGTTCGTTTATCGGG
TATTTTAGTCGCGTAGAAGGCGGAAGTTACGCGCGAGGGATCGC
GGTTCGTTCGGGATTAGTTTTAGGTTCGGTATCGTTTCGCGGGT
CGAGCGTTTATATTCGTTAAATTTACGCGGGTACGTTTTCGCGG
CGTATCGTTTTTAGTT;
and
the negative standard for detecting NDRG4 methylation comprises a polynucleotide sequence of
(SEQ ID NO. 70)
TGAGAAGTTGGTGGGGGTGTGGATTGATTGGGGTGTTTTTTAGG
TTTTGTGTTGTGGTTTTTGTTTGTTTTTTTGTTTGTTTATTGGG
TATTTTAGTTGTGTAGAAGGTGGAAGTTATGTGTGAGGGATTGT
GGTTTGTTTGGGATTAGTTTTAGGTTTGGTATTGTTTTGTGGGT
TGAGTGTTTATATTTGTTAAATTTATGTGGGTATGTTTTTGTGG
TGTATTGTTTTTAGTT.

21. The kit of claim 13, the means for amplifying an internal control gene comprises comprising primers for amplifying a positive control gene and/or a negative control gene.

22. A method for detecting the presence or absence of colorectal cancer (CRC) or advanced adenoma (AA) in a patient in need thereof, comprising:
a) obtaining genomic DNA from a biological sample of the patient;
b) treating the genomic DNA of a), or a fragment thereof, with one or more reagents to convert cytosine bases that are unmethylated thereof to uracil or another base that is detectably dissimilar to cytosine in terms of hybridization properties;
c) contacting the treated genomic DNA, or the treated fragment thereof, with a first pair of primers for detecting the presence or absence of methylation sites of a gene encoding bone morphogenetic protein 3 (BMP3) in the patient, and a second pair of primers for detecting the presence or absence of methylation sites of a gene encoding NDRG family member 4 protein (NDRG4) in the patient, wherein the first pair of primers comprise a contiguous sequence of at least 9 nucleotides that is identical to, complementary to, or hybridizes under stringent hybridization conditions to SEQ ID NO.: 1, and wherein the second pair of primers comprise a contiguous sequence of at least 9 nucleotides that is complementary to, or hybridizes under stringent hybridization conditions to SEQ ID NO.: 2, wherein the treated genomic DNA or the fragment thereof is either amplified to produce at least one amplificate by the first pair of primers or the second pair of primers, or is not amplified; and
d) determining the presence or absence of CRC or AA in the patient, based on a presence or absence of said amplificate, the methylation state or level of at least one CpG dinucleotide of the BMP3 gene and the NDRG4 gene in the patient.

23. The method of claim 22, wherein the first pair of primers and the first probe are selected from the group consisting of:
i) a forward primer comprising SEQ ID NO.: 3, a reverse primer comprising SEQ ID NO.: 4, and a probe comprising SEQ ID NO.: 5;
ii) a forward primer comprising SEQ ID NO.: 9, a reverse primer comprising SEQ ID NO.: 10, and a probe comprising SEQ ID NO.: 11, and
iii) a forward primer comprising SEQ ID NO.: 15, a reverse primer comprising SEQ ID NO.: 16, and a probe comprising SEQ ID NO.: 17, and,
wherein the second first pair of primers and the second probe are selected from the group consisting of:
iv) a forward primer comprising SEQ ID NO.: 6, a reverse primer comprising SEQ ID NO.: 7, and a probe comprising SEQ ID NO.: 8;
v) a forward primer comprising SEQ ID NO.: 12, a reverse primer comprising SEQ ID NO.: 13, and a probe comprising SEQ ID NO.: 14; and
vi) a forward primer comprising SEQ ID NO.: 18, a reverse primer comprising SEQ ID NO.: 19, and a probe comprising SEQ ID NO.: 20;

24. The method of claim 22, wherein the method comprises using
i) a forward primer comprising SEQ ID NO.: 3, a reverse primer comprising SEQ ID NO.: 4, and a probe comprising SEQ ID NO.: 5, for detecting the methylation state or level of at least one CpG dinucleotide of the BMP3 gene in the biological sample obtained from the patient, and
ii) a forward primer comprising SEQ ID NO.: 6, a reverse primer comprising SEQ ID NO.: 7, and a probe comprising SEQ ID NO.: 8, for detecting the methylation state or level of at least one CpG dinucleotide of the NDRG4 gene in the biological sample obtained from the patient.

25. The method of claim 22, wherein the method comprises using
i) a forward primer comprising SEQ ID NO.: 9, a reverse primer comprising SEQ ID NO.: 10, and a probe comprising SEQ ID NO.: 11, for detecting the methylation state or level of at least one CpG dinucleotide of the BMP3 gene in the biological sample obtained from the patient, and
ii) a forward primer comprising SEQ ID NO.: 12, a reverse primer comprising SEQ ID NO.: 13, and a probe comprising SEQ ID NO.: 14, for detecting the methylation state or level of at least one CpG dinucleotide of the NDRG4 gene in the biological sample obtained from the patient.

26. The method of claim 22, wherein the method comprises using
i) a forward primer comprising SEQ ID NO.: 15, a reverse primer comprising SEQ ID NO.: 16, and a probe comprising SEQ ID NO.: 17, for detecting the methylation state or level of at least one CpG dinucleotide of the BMP3 gene in the biological sample obtained from the patient, and
ii) a forward primer comprising SEQ ID NO.: 18, a reverse primer comprising SEQ ID NO.: 19, and a probe comprising SEQ ID NO.: 20, for detecting the methylation state or level of at least one CpG dinucleotide of the NDRG4 gene in the biological sample obtained from the patient.

27. The method of any one of claims 22 to 26, wherein both the first probe and the second probe comprise a fluorescent donor and an acceptor fluorophore.

28. The method of any one of claims 22 to 26, wherein first probe and the second probe are TAQMAN® probes.

29. The method of any one of claims 22 to 28, wherein the method further comprises a step of detecting the presence or absence of at least one mutation in the KRAS gene in a biological sample obtained from the patient, and a step of detecting the presence or absence of hemoglobin in a biological sample obtained from the patient.

30. The method of any one of claim 29, wherein the step of detecting the presence or absence of at least one mutation in the KRAS gene in the patient comprises using at least one pair of primers capable of amplifying the Exon 12 and/or Exon 13 region of the KRAS gene in a polymerase chain reaction (PCR).

31. The method of any one of claim 29, wherein the step of detecting the presence or absence of hemoglobin in the biological sample comprises using an anti-hemoglobin antibody.

32. The method of claim 30, wherein the primers are capable of amplifying a KRAS gene region comprising at least one KRAS mutation selected from the group consisting G12D, G12V, G12C, G13D, G12A, G12R, G12S, and G13C.

33. The method of claim 31, wherein the antibody is a colloidal gold-conjugated antibody.

34. The method of any one of claims 22 to 33, wherein the amplification of BMP3 gene is performed in a quantitative PCR (qPCR), and the method further comprises amplifying a first reference gene to determining the Ct value of the BMP3 amplification as ΔCt1.

35. The method of any one of claims 22 to 33, wherein the amplification of NDRG4 gene is performed in a quantitative PCR (qPCR), and the method further comprises amplifying a second reference gene to determining the Ct value of the NDRG4 amplification as ΔCt2.

36. The method of any one of claims 30 to 33, wherein the amplification of mutant KRAS gene is performed in a quantitative PCR (qPCR), and the method further comprises amplifying a third reference gene to determining the Ct value of the mutant KRAS amplification as ΔCt3.

37. The method of claim 34 or 35, wherein the first and the second reference genes are the same.

38. The method of claim 37, wherein the same reference gene is a B2M gene.

39. The method of claim 36, wherein the mutant KRAS gene comprises a mutation selected from the group consisting of G12D, G13D, G12V, G12C, G12S, G12A, and G13R.

40. The method of claim 39, wherein the mutant KRAS gene is amplified by one or more pairs of primers selected from the group consisting of:
(1) a forward primer G12D-F comprising SEQ ID NO.: 35, and a reverse primer Kras-R comprising SEQ ID NO.: 42;
(2) a forward primer G13D-F comprising SEQ ID NO.: 36, and a reverse primer Kras-R comprising SEQ ID NO.: 42;
(3) a forward primer G12V-F comprising SEQ ID NO.: 37, and a reverse primer Kras-R comprising SEQ ID NO.: 42;
(4) a forward primer G12C-F comprising SEQ ID NO.: 38, and a reverse primer Kras-R comprising SEQ ID NO.: 42;
(5) a forward primer G12S-F comprising SEQ ID NO.: 39, and a reverse primer Kras-R comprising SEQ ID NO.: 42;
(6) a forward primer G12A-F comprising SEQ ID NO.: 40, and a reverse primer Kras-R comprising SEQ ID NO.: 42; and
(7) a forward primer G12R-F comprising SEQ ID NO.: 41, and a reverse primer Kras-R comprising SEQ ID NO.: 42,
and wherein the KRAS probe for the qPCR comprises SEQ ID NO.: 46.

41. The method of claim 36, wherein the third reference gene is an ACTB gene.

42. The method of claim 41, wherein qPCR primers for amplifying ACTB gene comprise SEQ ID NOs.: 43 and 44, and the probe comprise SEQ ID NO.: 46.

43. The method of claims 22 to 42, wherein the method comprises using
(1) a positive standard and a negative standard for detecting BMP3 methylation in the sample, and (2) a positive standard and a negative standard for detecting NDRG4 methylation in the sample.

44. The method of claim 43, wherein
the positive standard for detecting BMP3 methylation comprises a polynucleotide sequence of
(SEQ ID NO: 67)
GTTAGTTTGGTCGGGTGTTTTTAAAAATAAAGCGAGGAGGGAAG
GTATAGATAGATTTTGAAAATATTCGGGTTATATACGTCGCGAT
TTATAGTTTTTTTTTAGCGTTGGAGTGGAGACGGCGTTCGTAGC
GTTTTGCGCGGGTGAGGTTCGCGTAGTTGTTGGGGAAGAGTTTA
TTTGTTAGGTTGCGTTGGGTTAGCGTAGTAAGTGGGGTTGGTCG
TTATTTCGTTGTATTCGGTCGCGTTTCGGGTTTCGTGCGTTTTC
GTTTTAG;
the negative standard for detecting BMP3 methylation comprises a polynucleotide sequence of
(SEQ ID NO: 68)
GTTAGTTTGGTTGGGTGTTTTTAAAAATAAAGTGAGGAGGGAAG
GTATAGATAGATTTTGAAAATATTTGGGTTATATATGTTGTGAT
TTATAGTTTTTTTTTAGTGTTGGAGTGGAGATGGTGTTTGTAGT
GTTTTGTGTGGGTGAGGTTTGTGTAGTTGTTGGGGAAGAGTTTA
TTTGTTAGGTTGTGTTGGGTTAGTGTAGTAAGTGGGGTTGGTTG
TTATTTTGTTGTATTTGGTTGTGTTTTGGGTTTTGTGTGTTTTT
GTTTTAG;
the positive standard for detecting NDRG4 methylation comprises a polynucleotide sequence of
(SEQ ID NO: 69)
TGAGAAGTCGGCGGGGGCGCGGATCGATCGGGGTGTTTTTTAGG
TTTCGCGTCGCGGTTTTCGTTCGTTTTTTCGTTCGTTTATCGGG
TATTTTAGTCGCGTAGAAGGCGGAAGTTACGCGCGAGGGATCGC
GGTTCGTTCGGGATTAGTTTTAGGTTCGGTATCGTTTCGCGGGT
CGAGCGTTTATATTCGTTAAATTTACGCGGGTACGTTTTCGCGG
CGTATCGTTTTTAGTT;
and
the negative standard for detecting NDRG4 methylation comprises a polynucleotide sequence of
(SEQ ID NO.: 70)
TGAGAAGTTGGTGGGGGTGTGGATTGATTGGGGTGTTTTTTAGG
TTTTGTGTTGTGGTTTTTGTTTGTTTTTTTGTTTGTTTATTGGG
TATTTTAGTTGTGTAGAAGGTGGAAGTTATGTGTGAGGGATTGT
GGTTTGTTTGGGATTAGTTTTAGGTTTGGTATTGTTTTGTGGGT
TGAGTGTTTATATTTGTTAAATTTATGTGGGTATGTTTTTGTGG
TGTATTGTTTTTAGTT.

45. The method of any one of claims 22 to 44, wherein the method comprises amplifying a quality control standard.

46. A method for detecting the presence or absence of colorectal cancer (CRC) or advanced adenoma (AA) in a patient in need thereof, comprising using a kit of any one of claims 1 to 21.

47. A method for detecting the presence or absence of colorectal cancer (CRC) or advanced adenoma (AA) in a patient in need thereof, comprising:
a) obtaining an untreated genomic DNA from a fecal sample of the patient;
b) treating the genomic DNA of a), or a fragment thereof, with one or more reagents to convert cytosine bases that are unmethylated thereof to uracil or another base that is detectably dissimilar to cytosine in terms of hybridization properties;
c) performing a quantitative PCR (qPCR) using the treated genomic DNA of b) as a template, and determining the Ct value of BMP3 gene in the patient as ΔCt1;
d) performing a qPCR using the treated genomic DNA of b) as a template, and determining the Ct value of NDRG4 gene in the patient as ΔCt2;
e) performing a qPCR using the untreated genomic DNA as a template, and determining the Ct value of a mutant KRAS gene in the patient as ΔCt3;
f) performing a fecal immunochemical test of hemoglobin protein in the fecal sample and determining a score as FIT;
g) determining the value of K, wherein K=a*ΔCt+b*ΔCt2+c*ΔCt3+d*FIT+X, wherein a, b, c, d, X are clinical constants; and
h) determining the value of a comprehensive index P, wherein P=e^{K}/(1+e^{K}), wherein e is the natural constant, wherein when P is equal or more than a predetermined threshold, the patient is determined to have CRC and/or AA, and when P is less than the threshold, the patient is determined to be health.

48. The method of claim 47, wherein the qPCR for amplifying BMP3 gene comprises a first pair of primers and a first probe, wherein the first pair of primers and the first probe are selected from the group consisting of:
i) a forward primer comprising SEQ ID NO.: 3, a reverse primer comprising SEQ ID NO.: 4, and a probe comprising SEQ ID NO.: 5;
ii) a forward primer comprising SEQ ID NO.: 9, a reverse primer comprising SEQ ID NO.: 10, and a probe comprising SEQ ID NO.: 11; and
iii) a forward primer comprising SEQ ID NO.: 15, a reverse primer comprising SEQ ID NO.: 16, and a probe comprising SEQ ID NO.: 17;
and,
wherein the qPCR for amplifying NDRG4 gene comprises a second pair of primers and a second probe, wherein the second pair of primers and the second probe are selected from the group consisting of:
iv) a forward primer comprising SEQ ID NO.: 6, a reverse primer comprising SEQ ID NO.: 7, and a probe comprising SEQ ID NO.: 8;
v) a forward primer comprising SEQ ID NO.: 12, a reverse primer comprising SEQ ID NO.: 13, and a probe comprising SEQ ID NO.: 14; and
vi) a forward primer comprising SEQ ID NO.: 18, a reverse primer comprising SEQ ID NO.: 19, and a probe comprising SEQ ID NO.: 20;

49. The method of claim 47, wherein the method comprises using
i) a forward primer comprising SEQ ID NO.: 3, a reverse primer comprising SEQ ID NO.: 4, and a probe comprising SEQ ID NO.: 5, for detecting the methylation state or level of at least one CpG dinucleotide of the BMP3 gene in the sample, and
ii) a forward primer comprising SEQ ID NO.: 6, a reverse primer comprising SEQ ID NO.: 7, and a probe comprising SEQ ID NO.: 8, for detecting the methylation state or level of at least one CpG dinucleotide of the NDRG4 gene in the sample.

50. The method of claim 47, wherein the method comprises using
i) a forward primer comprising SEQ ID NO.: 9, a reverse primer comprising SEQ ID NO.: 10, and a probe comprising SEQ ID NO.: 11, for detecting the methylation state or level of at least one CpG dinucleotide of the BMP3 gene in the sample, and
ii) a forward primer comprising SEQ ID NO.: 12, a reverse primer comprising SEQ ID NO.: 13, and a probe comprising SEQ ID NO.: 14, for detecting the methylation state or level of at least one CpG dinucleotide of the NDRG4 gene in the sample.

51. The method of claim 47, wherein the method comprises using
i) a forward primer comprising SEQ ID NO.: 15, a reverse primer comprising SEQ ID NO.: 16, and a probe comprising SEQ ID NO.: 17, for detecting the methylation state or level of at least one CpG dinucleotide of the BMP3 gene in the sample, and
ii) a forward primer comprising SEQ ID NO.: 18, a reverse primer comprising SEQ ID NO.: 19, and a probe comprising SEQ ID NO.: 20, for detecting the methylation state or level of at least one CpG dinucleotide of the NDRG4 gene in the sample.

52. The method of any one of claims 47 to 51, wherein both the first probe and the second probe comprise a fluorescent donor and an acceptor fluorophore.

53. The method of any one of claims 47 to 52, wherein first probe and the second probe are TAQMAN® probes.

54. The method of any one of claims 47 to 53, wherein the mutant KRAS gene comprising at least one KRAS mutation selected from the group consisting G12D, G12V, G12C, G13D, G12A, G12R, G12S, and G13C.

55. The method of any one of claims 47 to 54, wherein the fecal immunochemical test comprises a colloidal gold-conjugated antibody.

56. the method of any one of claims 47 to 55, wherein step c) and step d) comprises using B2M gene as a reference gene.

57. The method of claim 54, wherein the mutant KRAS gene is amplified by one or more pairs of primers selected from the group consisting of:
(1) a forward primer G12D-F comprising SEQ ID NO.: 35, and a reverse primer Kras-R comprising SEQ ID NO.: 42;
(2) a forward primer G13D-F comprising SEQ ID NO.: 36, and a reverse primer Kras-R comprising SEQ ID NO.: 42;
(3) a forward primer G12V-F comprising SEQ ID NO.: 37, and a reverse primer Kras-R comprising SEQ ID NO.: 42;
(4) a forward primer G12C-F comprising SEQ ID NO.: 38, and a reverse primer Kras-R comprising SEQ ID NO.: 42;
(5) a forward primer G12S-F comprising SEQ ID NO.: 39, and a reverse primer Kras-R comprising SEQ ID NO.: 42;
(6) a forward primer G12A-F comprising SEQ ID NO.: 40, and a reverse primer Kras-R comprising SEQ ID NO.: 42; and
(7) a forward primer G12R-F comprising SEQ ID NO.: 41, and a reverse primer Kras-R comprising SEQ ID NO.: 42,
and wherein the KRAS probe for the qPCR comprises SEQ ID NO.: 46.

58. The method of claim 57, wherein ACTB gene is used as a reference gene in the qPCR for amplifying the mutant KRAS gene.

59. The method of claim 58, wherein the qPCR primers for amplifying ACTB gene comprise SEQ ID NOs.: 43 and 44, and the qPCR probe for ACTB gene comprises SEQ ID NO.: 46.

60. The method of claims 47 to 59, wherein the method comprises using
(1) a positive standard and a negative standard for detecting BMP3 methylation in the sample, and
(2) a positive standard and a negative standard for detecting NDRG4 methylation in the sample.

61. The method of claim 60, wherein
the positive standard for detecting BMP3 methylation comprises a polynucleotide sequence of
(SEQ ID NO: 67)
GTTAGTTTGGTCGGGTGTTTTTAAAAATAAAGCGAGGAGGGAAG
GTATAGATAGATTTTGAAAATATTCGGGTTATATACGTCGCGAT
TTATAGTTTTTTTTTAGCGTTGGAGTGGAGACGGCGTTCGTAGC
GTTTTGCGCGGGTGAGGTTCGCGTAGTTGTTGGGGAAGAGTTTA
TTTGTTAGGTTGCGTTGGGTTAGCGTAGTAAGTGGGGTTGGTCG
TTATTTCGTTGTATTCGGTCGCGTTTCGGGTTTCGTGCGTTTTC
GTTTTAG;
the negative standard for detecting BMP3 methylation comprises a polynucleotide sequence of
(SEQ ID NO: 68)
GTTAGTTTGGTTGGGTGTTTTTAAAAATAAAGTGAGGAGGGAAG
GTATAGATAGATTTTGAAAATATTTGGGTTATATATGTTGTGAT
TTATAGTTTTTTTTTAGTGTTGGAGTGGAGATGGTGTTTGTAGT
GTTTTGTGTGGGTGAGGTTTGTGTAGTTGTTGGGGAAGAGTTTA
TTTGTTAGGTTGTGTTGGGTTAGTGTAGTAAGTGGGGTTGGTTG
TTATTTTGTTGTATTTGGTTGTGTTTTGGGTTTTGTGTGTTTTT
GTTTTAG;
the positive standard for detecting NDRG4 methylation comprises a polynucleotide sequence of
(SEQ ID NO.: 69)
TGAGAAGTCGGCGGGGGCGCGGATCGATCGGGGTGTTTTTTAGG
TTTCGCGTCGCGGTTTTCGTTCGTTTTTTCGTTCGTTTATCGGG
TATTTTAGTCGCGTAGAAGGCGGAAGTTACGCGCGAGGGATCGC
GGTTCGTTCGGGATTAGTTTTAGGTTCGGTATCGTTTCGCGGGT
CGAGCGTTTATATTCGTTAAATTTACGCGGGTACGTTTTCGCGG
CGTATCGTTTTTAGTT
and
the negative standard for detecting NDRG4 methylation comprises a polynucleotide sequence of
(SEQ ID NO.: 70)
TGAGAAGTTGGTGGGGGTGTGGATTGATTGGGGTGTTTTTTAGG
TTTTGTGTTGTGGTTTTTGTTTGTTTTTTTGTTTGTTTATTGGG
TATTTTAGTTGTGTAGAAGGTGGAAGTTATGTGTGAGGGATTGT
GGTTTGTTTGGGATTAGTTTTAGGTTTGGTATTGTTTTGTGGGT
TGAGTGTTTATATTTGTTAAATTTATGTGGGTATGTTTTTGTGG
TGTATTGTTTTTAGTT

62. The method of any one of claims 47 to 61, wherein the method comprises amplifying a quality control standard in the step c) and the step d).

63. A method for diagnosing and treating a colorectal cancer (CRC) and/or advanced adenoma (AA) in a patient in need thereof, comprising determining the presence or absence of CRC and/or AA in the patient by using a kit of any one of claims 1 to 21, and treating the patient depends on the presence or absence of CRC and/or AA in the patient.

64. A method for diagnosing and treating a colorectal cancer (CRC) and/or advanced adenoma (AA) in a patient in need thereof, comprising determining the presence or absence of CRC and/or AA in the patient by using a method of any one of claims 22 to 62, and treating the patient depends on the presence or absence of CRC and/or AA in the patient.


SEQUENCE LISTING (70 sequences)

SEQ ID NO: 1 - DNA, 271 bp, Homo sapiens
  modified_base (12)..(12): methylated
  modified_base (33)..(33): methylated
  modified_base (69)..(69): methylated
  modified_base (80)..(80): methylated
  modified_base (83)..(83): methylated
  modified_base (85)..(85): methylated
  modified_base (106)..(106): methylated
  modified_base (120)..(120): methylated
  modified_base (123)..(123): methylated
  modified_base (127)..(127): methylated
  modified_base (132)..(132): methylated
  modified_base (139)..(139): methylated
  modified_base (141)..(141): methylated
  modified_base (152)..(152): methylated
  modified_base (154)..(154): methylated
  modified_base (189)..(189): methylated
  modified_base (200)..(200): methylated
  modified_base (219)..(219): methylated
  modified_base (227)..(227): methylated
  modified_base (236)..(236): methylated
  modified_base (240)..(240): methylated
  modified_base (242)..(242): methylated
  modified_base (247)..(247): methylated
  modified_base (254)..(254): methylated
  modified_base (258)..(258): methylated
  modified_base (264)..(264): methylated
gttagtttggtcgggtgtttttaaaaataaagcgaggagggaaggtatagatagattttgaaaatattcgggttatatacgtcgcgatttatagtttttttttagcgttggagtggagacggcgttcgtagcgttttgcgcgggtgaggttcgcgtagttgttggggaagagtttatttgttaggttgcgttgggttagcgtagtaagtggggttggtcgttatttcgttgtattcggtcgcgtttcgggtttcgtgcgttttcgttttag

SEQ ID NO: 2 - DNA, 236 bp, Homo sapiens
  modified_base (9)..(9): methylated
  modified_base (12)..(12): methylated
  modified_base (18)..(18): methylated
  modified_base (20)..(20): methylated
  modified_base (25)..(25): methylated
  modified_base (29)..(29): methylated
  modified_base (48)..(48): methylated
  modified_base (50)..(50): methylated
  modified_base (53)..(53): methylated
  modified_base (55)..(55): methylated
  modified_base (62)..(62): methylated
  modified_base (66)..(66): methylated
  modified_base (74)..(74): methylated
  modified_base (78)..(78): methylated
  modified_base (85)..(85): methylated
  modified_base (98)..(98): methylated
  modified_base (100)..(100): methylated
  modified_base (109)..(109): methylated
  modified_base (118)..(118): methylated
  modified_base (120)..(120): methylated
  modified_base (122)..(122): methylated
  modified_base (130)..(130): methylated
  modified_base (132)..(132): methylated
  modified_base (137)..(137): methylated
  modified_base (141)..(141): methylated
  modified_base (159)..(159): methylated
  modified_base (165)..(165): methylated
  modified_base (170)..(170): methylated
  modified_base (172)..(172): methylated
  modified_base (177)..(177): methylated
  modified_base (181)..(181): methylated
  modified_base (191)..(191): methylated
  modified_base (202)..(202): methylated
  modified_base (204)..(204): methylated
  modified_base (210)..(210): methylated
  modified_base (216)..(216): methylated
  modified_base (218)..(218): methylated
  modified_base (221)..(221): methylated
  modified_base (226)..(226): methylated
tgagaagtcggcgggggcgcggatcgatcggggtgttttttaggtttcgcgtcgcggttttcgttcgttttttcgttcgtttatcgggtattttagtcgcgtagaaggcggaagttacgcgcgagggatcgcggttcgttcgggattagttttaggttcggtatcgtttcgcgggtcgagcgtttatattcgttaaatttacgcgggtacgttttcgcggcgtatcgtttttagtt

SEQ ID NO: 3 - DNA, 29 bp, Homo sapiens
tttgaaaatattcgggttatatacgtcgc

SEQ ID NO: 4 - DNA, 28 bp, Homo sapiens
ataaactcttccccaacaactacgcgaa

SEQ ID NO: 5 - DNA, 25 bp, Homo sapiens
agcgttggagtggagacggcgttcg

SEQ ID NO: 6 - DNA, 26 bp, Homo sapiens
atcgatcggggtgttttttaggtttc

SEQ ID NO: 7 - DNA, 26 bp, Homo sapiens
ccttctacgcgactaaaatacccgat

SEQ ID NO: 8 - DNA, 31 bp, Homo sapiens
cgtcgcggttttcgttcgttttttcgttcgt

SEQ ID NO: 9 - DNA, 25 bp, Homo sapiens
aatattcgggttatatacgtcgcga

SEQ ID NO: 10 - DNA, 28 bp, Homo sapiens
gcaacctaacaaataaactcttccccaa

SEQ ID NO: 11 - DNA, 26 bp, Homo sapiens
tggagtggagacggcgttcgtagcgt

SEQ ID NO: 12 - DNA, 17 bp, Homo sapiens
gcgggtgagaagtcggc

SEQ ID NO: 13 - DNA, 20 bp, Homo sapiens
gtaacttccgccttctacgc

SEQ ID NO: 14 - DNA, 25 bp, Homo sapiens
taggtttcgcgtcgcggttttcgtt

SEQ ID NO: 15 - DNA, 27 bp, Homo sapiens
aatattcgggttatatacgtcgcgatt

SEQ ID NO: 16 - DNA, 21 bp, Homo sapiens
acttactacgctaacccaacg

SEQ ID NO: 17 - DNA, 28 bp, Homo sapiens
tagcgttggagtggagacggcgttcgta

SEQ ID NO: 18 - DNA, 21 bp, Homo sapiens
cggttttcgttcgttttttcg

SEQ ID NO: 19 - DNA, 26 bp, Homo sapiens
aacctaaaactaatcccgaacgaacc

SEQ ID NO: 20 - DNA, 28 bp, Homo sapiens
tcgtttatcgggtattttagtcgcgtag

SEQ ID NO: 21 - DNA, 20 bp, Homo sapiens
gagtggagacggcgttcgta

SEQ ID NO: 22 - DNA, 23 bp, Homo sapiens
ccacttactacgctaacccaacg

SEQ ID NO: 23 - DNA, 26 bp, Homo sapiens
cgggtgaggttcgcgtagttgttggg

SEQ ID NO: 24 - DNA, 23 bp, Homo sapiens
gggtgttttttaggtttcgcgtc

SEQ ID NO: 25 - DNA, 21 bp, Homo sapiens
cgtaacttccgccttctacgc

SEQ ID NO: 26 - DNA, 40 bp, Homo sapiens
acgcgactaaaatacccgataaacgaacgaaaaaacgaac

SEQ ID NO: 27 - DNA, 21 bp, Homo sapiens
ttaggttgcgttgggttagcg

SEQ ID NO: 28 - DNA, 22 bp, Homo sapiens
actccgaaaacgcaaaaaaccg

SEQ ID NO: 29 - DNA, 26 bp, Homo sapiens
attcggtcgcgtttcgggtttcgtgc

SEQ ID NO: 30 - DNA, 19 bp, Homo sapiens
gacccgcgaaacgataccg

SEQ ID NO: 31 - DNA, 21 bp, Homo sapiens
tattcgggttatatacgtcgc

SEQ ID NO: 32 - DNA, 20 bp, Homo sapiens
cttactacgctaacccaacg

SEQ ID NO: 33 - DNA, 26 bp, Homo sapiens
cccaacaactacgcgaacctcacccg

SEQ ID NO: 34 - DNA, 20 bp, Homo sapiens
tcgcgcgtaacttccgcctt

SEQ ID NO: 35 - DNA, 22 bp, Homo sapiens
aacttgtggtagttggaggtga

SEQ ID NO: 36 - DNA, 25 bp, Homo sapiens
aacttgtggtagttggagctgggga

SEQ ID NO: 37 - DNA, 22 bp, Homo sapiens
aacttgtggtagttggagttgt

SEQ ID NO: 38 - DNA, 22 bp, Homo sapiens
aaacttgtggtagttggggctt

SEQ ID NO: 39 - DNA, 22 bp, Homo sapiens
aaacttgtggtagttggtgcta

SEQ ID NO: 40 - DNA, 22 bp, Homo sapiens
aacttgtggtagttggagcagc

SEQ ID NO: 41 - DNA, 22 bp, Homo sapiens
aaacttgtggtagttggagctc

SEQ ID NO: 42 - DNA, 24 bp, Homo sapiens
gaatggtcctgcaccagtaatatg

SEQ ID NO: 43 - DNA, 21 bp, Homo sapiens
agggcttcttgtcctttcctt

SEQ ID NO: 44 - DNA, 20 bp, Homo sapiens
cgtgctcgatggggtacttc

SEQ ID NO: 45 - DNA, 26 bp, Homo sapiens
aggcaagagtgccttgacgatacagc

SEQ ID NO: 46 - DNA, 27 bp, Homo sapiens
cgtgatggtgggcatgggtcagaagga

SEQ ID NO: 47 - DNA, 20 bp, Homo sapiens
agtttggtgtaagttaagag

SEQ ID NO: 48 - DNA, 21 bp, Homo sapiens
ctaactctattttaaacrcca

SEQ ID NO: 49 - DNA, 23 bp, Homo sapiens
gttttaatttttggaaaaggtaa

SEQ ID NO: 50 - DNA, 21 bp, Homo sapiens
acctaacaaataaactcttcc

SEQ ID NO: 51 - DNA, 22 bp, Homo sapiens
gaaggtatagatagattttgaa

SEQ ID NO: 52 - DNA, 24 bp, Homo sapiens
cacctaacacaactttacraaact

SEQ ID NO: 53 - DNA, 26 bp, Homo sapiens
gtatttagttatggttggggygagta

SEQ ID NO: 54 - DNA, 21 bp, Homo sapiens
ctcacctactactaccgcccr

SEQ ID NO: 55 - DNA, 25 bp, Homo sapiens
aggtttttgagtttttggttttttt

SEQ ID NO: 56 - DNA, 20 bp, Homo sapiens
ccctccaaaccccctataac

SEQ ID NO: 57 - DNA, 20 bp, Homo sapiens
ggatggggatgtttttgtag

SEQ ID NO: 58 - DNA, 20 bp, Homo sapiens
rgrgaaacctaaaaaacacc

SEQ ID NO: 59 - DNA, 18 bp, Homo sapiens
gyggagygggtgagaagt

SEQ ID NO: 60 - DNA, 20 bp, Homo sapiens
craacaaccaaaaacccctc

SEQ ID NO: 61 - DNA, 23 bp, Homo sapiens
gttygttygggattagttttagg

SEQ ID NO: 62 - DNA, 19 bp, Homo sapiens
crcaaacraaaaacraaac

SEQ ID NO: 63 - DNA, 18 bp, Homo sapiens
gyggygttttygtttttg

SEQ ID NO: 64 - DNA, 20 bp, Homo sapiens
cracractaaaaatccccaa

SEQ ID NO: 65 - DNA, 271 bp, Homo sapiens
  modified_base (12)..(12): methylated
  modified_base (33)..(33): methylated
  modified_base (69)..(69): methylated
  modified_base (80)..(80): methylated
  modified_base (83)..(83): methylated
  modified_base (85)..(85): methylated
  modified_base (106)..(106): methylated
  modified_base (120)..(120): methylated
  modified_base (123)..(123): methylated
  modified_base (132)..(132): methylated
  modified_base (139)..(139): methylated
  modified_base (141)..(141): methylated
  modified_base (152)..(152): methylated
  modified_base (154)..(154): methylated
  modified_base (189)..(189): methylated
  modified_base (200)..(200): methylated
  modified_base (219)..(219): methylated
  modified_base (227)..(227): methylated
  modified_base (240)..(240): methylated
  modified_base (242)..(242): methylated
  modified_base (247)..(247): methylated
  modified_base (254)..(254): methylated
  modified_base (258)..(258): methylated
  modified_base (264)..(264): methylated
gccagtttggccgggtgttcccaaaaataaagcgaggagggaaggtacagacagatcttgaaaacacccgggccacacacgccgcgacctacagctctttctcagcgttggagtggagacggcgcccgcagcgccctgcgcgggtgaggtccgcgcagctgctggggaagagcccacctgtcaggctgcgctgggtcagcgcagcaagtggggctggccgctatctcgctgcacccggccgcgtcccgggctccgtgcgccctcgccccag

SEQ ID NO: 66 - DNA, 236 bp, Homo sapiens
  modified_base (9)..(9): methylated
  modified_base (12)..(12): methylated
  modified_base (18)..(18): methylated
  modified_base (20)..(20): methylated
  modified_base (25)..(25): methylated
  modified_base (29)..(29): methylated
  modified_base (48)..(48): methylated
  modified_base (50)..(50): methylated
  modified_base (53)..(53): methylated
  modified_base (55)..(55): methylated
  modified_base (62)..(62): methylated
  modified_base (66)..(66): methylated
  modified_base (74)..(74): methylated
  modified_base (78)..(78): methylated
  modified_base (85)..(85): methylated
  modified_base (98)..(98): methylated
  modified_base (100)..(100): methylated
  modified_base (109)..(109): methylated
  modified_base (118)..(118): methylated
  modified_base (120)..(120): methylated
  modified_base (122)..(122): methylated
  modified_base (130)..(130): methylated
  modified_base (132)..(132): methylated
  modified_base (137)..(137): methylated
  modified_base (141)..(141): methylated
  modified_base (159)..(159): methylated
  modified_base (165)..(165): methylated
  modified_base (170)..(170): methylated
  modified_base (172)..(172): methylated
  modified_base (177)..(177): methylated
  modified_base (181)..(181): methylated
  modified_base (191)..(191): methylated
  modified_base (202)..(202): methylated
  modified_base (204)..(204): methylated
  modified_base (210)..(210): methylated
  modified_base (216)..(216): methylated
  modified_base (218)..(218): methylated
  modified_base (221)..(221): methylated
  modified_base (226)..(226): methylated
tgagaagtcggcgggggcgcggatcgaccggggtgtcccccaggctccgcgtcgcggtccccgctcgccctcccgcccgcccaccgggcaccccagccgcgcagaaggcggaagccacgcgcgagggaccgcggtccgtccgggactagccccaggcccggcaccgccccgcgggccgagcgcccacacccgccaaacccacgcgggcacgcccccgcggcgcaccgcccccagcc

SEQ ID NO: 67 - DNA, 271 bp, Homo sapiens; identical to SEQ ID NO: 1

SEQ ID NO: 68 - DNA, 271 bp, Homo sapiens
gttagtttggttgggtgtttttaaaaataaagtgaggagggaaggtatagatagattttgaaaatatttgggttatatatgttgtgatttatagtttttttttagtgttggagtggagatggtgtttgtagtgttttgtgtgggtgaggtttgtgtagttgttggggaagagtttatttgttaggttgtgttgggttagtgtagtaagtggggttggttgttattttgttgtatttggttgtgttttgggttttgtgtgtttttgttttag

SEQ ID NO: 69 - DNA, 236 bp, Homo sapiens; identical to SEQ ID NO: 2

SEQ ID NO: 70 - DNA, 236 bp, Homo sapiens
tgagaagttggtgggggtgtggattgattggggtgttttttaggttttgtgttgtggtttttgtttgtttttttgtttgtttattgggtattttagttgtgtagaaggtggaagttatgtgtgagggattgtggtttgtttgggattagttttaggtttggtattgttttgtgggttgagtgtttatatttgttaaatttatgtgggtatgtttttgtggtgtattgtttttagtt